(19) United States
(12) Patent Application Publication
(10) Pub. No.: US 2009/0092650 A1
(43) Pub. Date: Apr. 9, 2009

(54) Sustained Delivery Formulations of Octreotide Compounds

(76) Inventors: Stephen L. Warren, Fort Collins, CO (US); Eric Dadey, Furlong, PA (US); Richard L. Dunn, Fort Collins, CO (US); John Milton Downing, Fort Collins, CO (US); Ellen Qi Li, Ft. Collins, CO (US)

Correspondence Address: SCHWEGMAN, LUNDBERG & WOESSNER, P.A., P.O. BOX 2938, MINNEAPOLIS, MN, 55402, US

(21) Appl. No.: 11/793,296

(22) PCT Filed: Dec. 15, 2005
(86) PCT No.: PCT/US05/45346

§ 371 Date: Jul. 31, 2008

Related U.S. Application Data
(60) Provisional application No. 60/636,273, filed on Dec. 15, 2004.

(51) Int. Cl. A61K 9/00; A61K 38/08; A61P 3/10; A61P 35/00

(52) U.S. Cl. 424/422; 514/16

(57) ABSTRACT

The present invention relates to an octreotide sustained release delivery system for treatment of diseases relating to somatotropin and/or somatostatin. The sustained release delivery system of the invention includes a flowable composition containing an octreotide compound, and an implant containing the octreotide compound. The flowable composition may be injected into tissue whereupon it coagulates to become the solid or gel, monolithic implant. The flowable composition includes a biodegradable, thermoplastic polymer, an organic liquid and an octreotide compound.

### FIELD OF THE INVENTION

The present invention relates to an octreotide sustained release delivery system for treatment of diseases ameliorated by octreotide compounds. The sustained release delivery system of the invention includes a flowable composition containing octreotide, and an implant containing the octreotide.

### BACKGROUND OF THE INVENTION

Although the treatment of all malconditions relating to somatostatin and somatotropin are within the scope of the invention, a discussion of ocular disease resulting from diabetes is of particular interest.

Diabetic Retinopathy: One treatment of malconditions relating to somatostatin concerns the treatment of diabetic retinopathy. Diabetic retinopathy is the leading cause of blindness in patients between the ages of 25 to 74 years. It is estimated that diabetic retinopathy will be responsible for 12,000 to 24,000 new cases of blindness in the United States each year.

Diabetic retinopathy is subdivided into two main categories: non-proliferative and proliferative diabetic retinopathy. Nonproliferative diabetic retinopathy (NPDR) is characterized by intraretinal micro aneurysms, hemorrhages, nerve-fiber-layer infarcts, hard exudates, and microvascular abnormalities. Proliferative retinopathy (PDR) is characterized by neovascularization arising from the disk or from more peripheral retinal vessels.

Macular edema is the main cause of visual loss in nonproliferative retinopathy (NPDR). Macular edema results from focal vascular leakage from microaneurysms in the capillaries, as well as from diffuse vascular leakage.

The pathogenesis of retinal neovascularization in proliferative diabetic retinopathy is incompletelyunderstood. Current theories focus on the role of angiogenic factors (e.g. vascular endothelial growth factor, platelet derived growth factor and basic fibroblastic growth factor) produced by ischemic and hypoxic regions of the retina. It is believed that endogenous, hypoxia-induced angiogenic factors drive neovascular proliferation from retinal vessels.

Medical Treatments for Diabetic Retinopathy: Recent evidence suggests that octreotide has efficacy in two distinct diabetic retinopathy indications. The first indication is to reduce vitreous hemorrhage and loss of visual acuity in patients with high risk proliferative retinopathy (Boehm, B. O. et al. 1998). Another diabetic eye indication includes patients at earlier stages of the disease (Grant, M. B. et al, 2000). This includes severe nonproliferative (NPDR) and early proliferative diabetic retinopathy (ePDR).

The Sandostatin® product has been developed for treatment of diseases related to endogeneous somatostatin and/or somatotropin. One form is the Sandostatin LAR® depot, which is a sustained release composition of microparticles containing octreotide. Another is an injectable aqueous solution of octreotide, tradenamed Sandostatin® injection.

Recently, Sandostatin® injection has been studied as a treatment for diabetic retinopathy. Effective treatment of diabetic retinopathy using octreotide required multiple daily subcutaneous injections of Sandostatin® injection with total daily doses between 200 and 5,000 micrograms (Grant, M. B. et al, 2000). However, its use in this manner is plagued by such problems as large injection volumes, significant variation in blood level, lack of sustained blood level, multiple daily injection regimen and short duration of action. Consequently, there is a need for a product that provides higher and more consistent levels of octreotide (or another somatostatin-analogue) to treat diabetic retinopathy while minimizing these side effects.

Age-Related Macular Degeneration: A second treatment of malconditions relation to somatostatin concerns treatment of age-related macular degeneration (AMD). AMD includes the dry and wet forms. The wet form of AMD is responsible for substantial visual loss in the elderly. The Framingham Eye Study revealed that the overall prevalence of all kinds of AMD is 1.2 percent in patients 52 to 64 years old, increasing to 19.7 percent at 75 to 85 years of age. The Beaver Dam Eye Study revealed a prevalence of 36.8 percent in patients 75 years of age or older. The extent of visual loss and progression of disease are highly variable in AMD. The cause of AMD is unknown. However, genetic, nutritional, hemodynamic, degenerative, and phototoxic etiological factors are under investigation. The dry and wet forms may be entirely different diseases.

Treatment of Choroidal Neovascularization: There are two treatments for wet AMD: laser surgery and photodynamic therapy; however, neither treatment is a cure. Each treatment may slow the rate of vision decline or stop further vision loss. The disease and loss of vision may progress despite treatment.

Laser surgery involves the use of a thermal argon laser to destroy the fragile, leaky blood vessels. A high-energy laser beam is aimed directly onto the new blood vessels and destroys them, preventing further loss of vision. However, this kinds of laser treatment also may destroy some surrounding healthy tissue and some vision. Only a small percentage of people with wet AMD are candidates for laser surgery.

Photodynamic therapy is a much more common treatment. It involves the administration of verteporfin, a photosensitizing drug, and the subsequent application of a non-thermal light to the retina. The light activates the verteporfin molecule leading to destruction of the abnormal blood vessels. Verteporfin is injected intravenously, and circulates throughout the body and is sequestered in the neovessels of the eye. Verteporfin is taken up by the endothelial cells in the neovessels. Next, the affected eye is exposed to a 689 nm light for about 90 seconds. The light activates the drug thereby leading to the production of reactive-oxygen species, including superoxide. The activated drug destroys the new blood vessels and leads to a slower rate of vision decline. Treatments are usually administered at intervals of 3 months or more.

Unlike laser surgery, verteporfin does not destroy surrounding healthy tissue. Because the drug is activated by light, it is important for the patient to avoid exposure of the skin or eyes to direct sunlight or bright indoor light for five days after treatment. Photodynamic therapy is relatively painless, and is typically performed in the doctor's office in approximately 20 minutes. Although photodynamic therapy slows the rate of vision loss, it does not stop vision loss or restore vision in eyes already damaged by advanced AMD, and treatment results often are temporary. Photodynamic therapy is not the standard of care for wet AMD.

The most common cause of CNV lesions is AMD, but the development of CNV lesions is associated with many other diseases and conditions in the eye, including but not limited to pathologic myopia, presumed ocular histoplasmosis syndrome and angioid streaks.

Octreotide is a somatostatin analogue that binds preferentially to SSTR-2A, SSTR-3 and SSTR-5 receptor subtypes (Barnett, P. et al, 2003; Benali, N. et al, 2000; Culler, M. D. et al, 2002; McKreage, K. et al, 2003; Moller, L. N. et al, 2003; Patel, Y. C. et al, 1999; and Spraul, C. W. et al 2003). Pharmaceutical formulations of octreotide, i.e., Sandostatin (Injection and Sandostatin LAR®, are approved for the treatment of acromegaly (excessive production of growth hormone by the pituitary gland). These products are also approved for the symptomatic treatment of diarrhea associated with carcinoid syndrome and vasoactive intestinal peptide (VIP) tumors. Octreotide also has many “off label” uses, including the treatment of chemotherapy-induced diarrhea, Graves opthalmopathy, pancreatitis, bleeding esophageal varices, and ascites associated with portosystemic shunting in patients with cirrhosis.

It has been recognized for many years that octreotide and other somatostatin analogues have anti-angiogenic properties. These anti-vacularization effects are thought to be mediated by activation of SSTR-2A and SSTR-3, two receptor subtypes that are preferentially expressed in neovascular endothelial cells (Barnett, P. et al, 2003; Benali, N. et al, 2000; Culler, M. D. et al, 2002; Lambooij, A. C. et al 2000; McKreage, K. et al, 2003; Moller, L. N. et al, 2003; Patel, Y. C. et al, 1999; and Spraul, C. W. et al 2003; Woltering, E. A. et al, 2003). Furthermore, activation of SSTR-2A and SSTR-3 by somatostatin analogues inhibits both the proliferation and migration of endothelial cells.

Direct effects on SSTR-2A and SSTR-3 are believed to be the primary mechanism by which somatostatin analogues inhibit angiogenesis. However, the anti-angiogenic activity of somatostatin analogues may also involve indirect mechanisms. For example, somatostatins inhibit the production of growth hormone (GH) secretion by the pituitary gland, resulting in a reduction of insulin-like growth factor (IGF-1), which seems to have a permissive or stimulatory role in angiogenesis. Finally, in certain tissues somatostatin analogues are believed to inhibit the production of endogenous angiogenic factors, such as vascular endothelial growth factor (VEGF). Thus, the anti-angiogenic properties of somatostatin analogues have been widely recognized for many years.

Over the past decade, pharmaceutical research has been focused on improving the receptor selectivity of somatostatin analogues. In the field of opthalmological drug development, the goal has been to create analogues that bind more tightly and selectively to the SSTR-2A and SSTR-3 receptor subtypes. An equally important goal is to increase the bioavailability of somatostatin analogues. One approach to improve bioavailability is to create sustained release depot formulations that constantly release a somatostatin peptide analogue into the bloodstream for many weeks. The only currently marketed, sustained release somatostatin analogue is Sandostatin® LAR, which provides a 1-month release profile. A major limitation of this product, and other microsphere based products, is their relatively low bioavailability.

Therefore, there is a need to develop a product providing an increased bioavailability of octreotide and other somatostatin analogues. In particular, there is a need to develop sustained release formulations of somatostatin analogs that do not suffer from low bioavailability, poor release kinetics, injection site toxicity, relatively large volume injections and inconveniently short duration of release.

### SUMMARY OF THE INVENTION

The present invention is directed to an octreotide sustained release delivery system capable of delivering octreotide for a duration of about 14 days to about 3 months. The octreotide sustained release delivery system includes a flowable composition and a gel or solid implant for the sustained release of octreotide. The implant is produced from the flowable composition. In certain preferred embodiments, the octreotide sustained release delivery system provides in situ 1-month and 3-month release profiles characterized by an exceptionally high bioavailability and minimal risk of permanent tissue damage and essentially no risk of muscle necrosis.

Several direct comparisons between the octreotide sustained relesase delivery system of the invention and Sandostatin LAR® product have been conducted in the preclinical and clinical settings. In all cases, the octreotide sustained release delivery system of the invention provides significantly higher bioavailability of octreotide as compared to Sandostatin LAR® product. In addition, the sustained release delivery system of the invention provides blood levels in the therapeutic range immediately after injection, whereas Sandostatin LAR® product has exhibited the characteristic lag phase prior to the release of octreotide. Finally, the sustained release delivery system of the invention causes little or no tissue necrosis while the Sandostatin LARS product causes significant tissue necrosis.

The present invention is directed to an octreotide sustained release delivery system. This delivery system includes a flowable composition and a controlled, sustained release implant. The flowable composition of the invention includes a biodegradable thermoplastic polymer, a biocompatible, polar, aprotic organic liquid and octreotide. The flowable composition of the invention may be transformed into the implant of the invention by contact with water, body fluid or other aqueous medium. In one embodiment, the flowable composition is injected into the body whereupon it transforms in situ into the solid or gel implant of the invention.

The thermoplastic polymer of the flowable composition and implant is at least substantially insoluble in an aqueous medium or body fluid, preferably, essentially completely insoluble in those media. The thermoplastic polymer may be a homopolymer, a copolymer or a terpolymer of repeating monomeric units linked by such groups as ester groups, anhydride groups, carbonate groups, amide groups, urethane groups, urea groups, ether groups, esteramide groups, acetal groups, ketal groups, orthocarbonate groups and any other organic functional group that can be hydrolyzed by enzymatic or hydrolytic reaction (i.e., is biodegradable by this hydrolytic action). The preferred thermoplastic polymer, polyester, may be composed of units of one or more hydroxycarboxylic acid residues or diol and dicarboxylic acid residues, wherein the distribution of differing residues may be random, block, paired or sequential.

When the biodegradable thermoplastic polymer is a polyester, the preferable polyesters include a polylactide, a polyglycolide, a polycaprolactone, a copolymer thereof, a terpolymer thereof, or any combination thereof, optionally incorporating a third mono-alcohol or polyol component. More preferably, the biodegradable thermoplastic polyester is a polylactide, a polyglycolide, a copolymer thereof, a terpolymer thereof, or a combination thereof, optionally incorporating a third mono-alcohol or polyol component. More preferably, the suitable biodegradable thermoplastic polyester is 50/50 poly (lactide-co-glycolide) (hereinafter PLG) having a carboxy terminal group or is a 75/25 or a 85/15 PLG with a carboxy terminal group or such a PLG formulated with one or more mono-alcohol or polyol units. When a mono-alcohol or polyol is incorporated into the polyester, the mono-alcohol or polyol constitutes a third covalent component of the polymer chain. When a mono-alcohol is incorporated, the carboxy terminus of the polyester is esterified with the mono-alcohol. When a polyol is incorporated, it chain extends and optionally branches the polyester. The polyol functions as a polyester polymerization point with the polyester chains extending from multiple hydroxyl moieties of the polyol, and those hydroxyl moieties are esterified by a carboxyl group of the polyester chain. For an embodiment employing a diol, the polyester is linear with polyester chains extending from both esterified hydroxy groups. For an embodiment employing a triol or higher polyol, the polyester may be linear or may be branched with polyester chains extending from the esterified hydroxy groups. Examples of polyols include aliphatic and aromatic diols, saccharides such as glucose, lactose, maltose, sorbitol, triols such as glycerol, fatty alcohols and the like, tetraols, pentaols, hexaols and the like.

The biodegradable thermoplastic polymer can be present in any suitable amount, provided the biodegradable thermoplastic polymer is at least substantially insoluble in aqueous medium or body fluid. The biodegradable thermoplastic polymer is present in about 10 wt. % to about 95 wt. % of the flowable composition, preferably present in about 20 wt. % to about 70 wt. % of the flowable composition or more preferably is present in about 30 wt. % to about 60 wt. % of the flowable composition. Preferably, the biodegradable thermoplastic polymer has an average molecular weight of about 10,000 to about 45,000 or more preferably about 15,000 to about 35,000.

The flowable composition of the invention also includes a biocompatible, polar aprotic organic liquid. The biocompatible polar aprotic liquid can be an amide, an ester, a carbonate, a ketone, an ether, a sulfonyl or any other organic compound that is liquid at ambient temperature, is polar and is aprotic. The biocompatible polar aprotic organic liquid may be only very slightly soluble to completely soluble in all proportions in body fluid. While the organic liquid generally will have similar solubility profiles in aqueous medium and body fluid, body fluid is typically more lipophilic than aqueous medium. Consequently, some organic liquids that are insoluble in aqueous medium will be at least slightly soluble in body fluid. These examples of organic liquid are included within the definition of organic liquids according to the invention.

Preferably, the biocompatible polar aprotic liquid is N-methyl-2-pyrrolidone, 2-pyrrolidone, N,N-dimethylformamide, dimethyl sulfoxide, propylene carbonate, caprolactam, triacetin, or any combination thereof. More preferably, the biocompatible polar aprotic liquid is N-methyl-2-pyrrolidone. Preferably, the polar aprotic organic liquid is present in about 30 wt. % to about 80 wt. % of the composition or is present in about 40 wt. % to about 60 wt. % of the composition.

The flowable composition of the invention also includes octreotide compounds (hereinafter octreotide) which are oligopeptides having somatostatin-like properties. The octreotide is present in at least about a 0.1 wt. % concentration in the flowable composition with the upper limit being the limit of dispersibility of the peptide within the flowable composition. Preferably, the concentration is about 0.5 wt. % to about 20 wt. % of the flowable composition or more preferably about 1 wt. % to about 15 wt. % of the flowable composition.

Preferably, the flowable composition of the invention is formulated as an injectable delivery system. The flowable composition preferably has a volume of about 0.20 mL to about 2.0 mL or preferably about 0.30 mL to about 1.0 mL. The injectable composition is preferably formulated for administration about once per month, about once per three months, or about once per four months, to about once per six months. Preferably, the flowable composition is a liquid or a gel composition, suitable for injection into a patient.

Excipients, release modifiers, plasticizers, pore forming agents, gelation liquids, non-active extenders, and other ingredients may also be included within the octreotide sustained release delivery system of the invention. Upon administration of the flowable composition, some of these additional ingredients, such as gelation liquids and release modifiers will remain with the implant, while others, such as pore forming agents will separately disperse and/or diffuse along with the organic liquid.

The present invention also is directed to a method for forming a flowable composition. The method includes mixing, in any order, a biodegradable thermoplastic polymer, a biocompatible polar aprotic liquid, and octreotide. These ingredients, their properties, and preferred amounts are as disclosed above. The mixing is performed for a sufficient period of time effective to form the flowable composition for use as a controlled release implant. Preferably, the biocompatible thermoplastic polymer and the biocompatible polar aprotic organic liquid are mixed together to form a mixture and the mixture is then combined with the octreotide to form the flowable composition. Preferably, the flowable composition is a solution or dispersion, especially preferably a solution, of the octreotide and biodegradable thermoplastic polymer in the organic liquid. The flowable composition preferably includes an effective amount of a biodegradable thermoplastic polymer, an effective amount of a biocompatible polar aprotic organic liquid and an effective amount of octreotide. These ingredients, the preferred ingredients, their properties, and preferred amounts are as disclosed above.

The present invention also is directed to a method of forming a biodegradable implant in situ, in a living patient. The method includes injecting the flowable composition of the present invention within the body of a patient and allowing the biocompatible polar aprotic organic liquid to dissipate to produce a solid or gel biodegradable implant. Preferably, the biodegradable solid or gel implant releases an effective amount of octreotide by diffusion, erosion, or a combination of diffusion and erosion as the solid or gel implant biodegrades in the patient.

The present invention also is directed to a method of treating or preventing mammalian diseases that are ameloriated, cured or prevented by octreotide. The method includes administering, to a patient (preferably a human patient) in need of such treatment or prevention, an effective amount of a flowable composition of the present invention. Specifically, the diseases can be those that have an etiology associated with growth hormone related problems, including those concerning imbalance or malconditions associated with insulin, glucagon and/or somatotropin or somatostatin pathways. In particular, the diseases are those associated with diabetes including but not limited to cardioconditions, ocular conditions, nephritic conditions. Especially, these diseases include those concerning ocular conditions such as diabetic retinopathy and proliferative eye disease.

The present invention also is directed to a kit. The kit includes a first container and a second container. The first container includes a composition of the biodegradable thermoplastic polymer and the biocompatible polar aprotic organic liquid. The second container includes octreotide. These ingredients, their properties, and preferred amounts are as disclosed above. Preferably, the first container is a syringe and the second container is a syringe. In addition, the octreotide is preferably lyophilized. The kit can preferably include instructions. Preferably, the first container can be connected to the second container. More preferably, the first container and the second container are each configured to be directly connected to each other.

The present invention also is directed to a solid or gel implant. The solid or gel implant is composed of at least the biocompatible thermoplastic polymer and octreotide and is substantially insoluble in body fluid. While octreotide itself has at least some solubility in body fluid, its isolation within the substantially insoluble implant allows for its slow, sustained release into the body.

The solid implant has a solid matrix or a solid microporous matrix while the gel implant has a gelatinous matrix. The matrix can be a core surrounded by a skin. When microporous, the core preferably contains pores of diameters from about 1 to about 1000 microns. When microporous, the skin preferably contains pores of smaller diameters than those of the core pores. In addition, the skin pores are preferably of a size such that the skin is functionally non-porous in comparison with the core.

The solid or gel implant can optionally include one or more biocompatible organic substances which may function as an excipient as described above, or which may function as a plasticizer, a sustained release profile modifier, emulsifier and/or isolation carrier for octreotide.

The biocompatible organic liquid may also serve as an organic substance of the implant and/or may provide an additional function such as a plasticizer, a modifier, an emulsifier or an isolation carrier. There may be two or more organic liquids present in the flowable composition such that the primary organic liquid acts as a mixing, solubilizing or dispersing agent, and the supplemental organic liquid or liquids provide additional functions within the flowable composition and the implant. Alternatively, there may be one organic liquid which at least may act as a mixing, solubilizing or dispersing agent for the other components, and may provide additional functions as well. As second or additional components, additional kinds of biodegradable organic liquids typically are combined with the flowable composition and may remain with the implant as the administered flowable composition coagulates.

When serving as a plasticizer, the biocompatible organic substance provides such properties as flexibility, softness, moldability and drug release variation to the implant. When serving as a modifier, the biocompatible organic substance also provides the property of octreotide release variation to the implant. Typically, the plasticizer increases the rate of octreotide release while the modifier slows the rate of octreotide release. Also, there can be structural overlap between these two kinds of organic substances functioning as plasticizers and rate modifiers.

When serving as an emulsifier, the biocompatible organic substance at least in part enables a uniform mixture of the octreotide within the flowable composition and within the implant.

When serving as an isolation carrier, the biocompatible organic substance will function to encapsulate, isolate or otherwise surround molecules or nanoparticles of the octreotide so as to prevent its burst at least in part, and to isolate the octreotide from degradation by other components of the flowable composition and implant.

The amount of biocompatible organic substance optionally remaining in the solid or gel implant is preferably minor, such as from about 0 wt. % (or an almost negligible amount) to about 20 wt. % of the composition. In addition, the amount of biocompatible organic substance optionally present in the solid or gel implant preferably decreases over time.

### BRIEF DESCRIPTION OF THE DRAWINGS

FIG. 1: 84 Day Release Profile of ATRIGEL®/Octreotide Formulations Following Subcutaneous Injection in Rats; [symbol] GpI: 12% Octreotide acetate+citric acid in [45% 65/35 PLG (InV 0.36) and 55% NMP]; [symbol] GpII: 12% Octreotide acetate+citric acid in [50% 85/15 PLGH (InV 0.25) and 50% NMP]; [symbol] GpIII: 12% Octreotide acetate+citric acid in [25% 85/15 PLGH (InV 0.25)+25% 85/15 PLG (InV 0.25) and 50% NMP]; and [symbol] GpIV: 12% Octreotide acetate+citric acid in [30% 85/15 PLGH (InV 0.25)+20% 65/35 PLG (InV 0.36) and 50% NMP.

FIG. 2: Mean Octreotide Plasma Levels in Rats (Groups I and II) Following Subcutaneous Injection of ATRIGEL®/Octreotide Formulations; [symbol] GpI: 12% Octreotide acetate+citric acid in 45% 65/35 PLG (InV 0.36) /55% NMP; and [symbol] GpII: 12% Octreotide acetate+citric acid in 50% 85/15 PLGH (InV 0.25)/50% NMP.

FIG. 3: 85-Day Release Profiles of ATRIGEL®/Octreotide Formulations; [symbol] GpI: 12% Octreotide+citric acid in 50% 85/15 PLGH (InV 0.25); [symbol] GpII: 15% Octreotide+citric acid in 50% 85/15 PLGH (InV 0.25); [symbol] GpIII: 12% Octreotide+citric acid in 20% 85/15 PLGH (InV 0.25)+30% 65/35 PLG (InV 0.37); ⋄ GpIV: 12% Octreotide+citric acid in 30% 85/15 PLGH (InV 0.25)+20% 65/35 PLG (InV 0.37); [symbol] GpV: 12% Octreotide+citric acid in 35% 85/15 PLGH (InV 0.25)+15% 65/35 PLG (InV 0.37); and [symbol] GpVI: 12% Octreotide+citric acid in 30% 85/15 PLGH (InV 0.25)+20% 50/50 PLGH (InV 0.30).

FIG. 4: Plasma Octreotide Levels in Rats (Groups I and II); [symbol] GpI: 12% Octreotide+citric acid in 50% 85/15 PLGH (InV 0.25)/50% NMP; and [symbol] GpII: 15% Octreotide+citric acid in 50% 85/15 PLGH (InV 0.25)/50% NMP.

FIG. 5: 99-Day Release Profile of ATRIGEL®/Octreotide Formulations Following Subcutaneous Administration in Rats; [symbol] Group I: 12% Octreotide acetate+citric acid in (50% 85/15 PLGH (InV 0.27)/50% NMP); [symbol] Group II: 13.5% Octreotide acetate+citric acid in (50% 85/15 PLGH (InV 0.27)/50% NMP; [symbol] Group III: 15% Octreotide acetate+citric acid in (50% 85/15 PLGH (InV 0.27)/50% NMP; and Reference.

FIG. 6: Pharmacokinetic Profile ATRIGEL®/Octreotide Formulations Following Subcutaneous Administration in Rats; [symbol] GpI: 12% Octreotide acetate+citric acid in (50% 85/15 PLGH (InV 0.27)/50% NMP); [symbol] GpII: 13.5% Octreotide acetate+citric acid in (50% 85/15 PLGH (InV 0.27)/50% NMP); and [symbol] GpIII: 15% Octreotide acetate+citric acid in (50% 85/15 PLGH (InV 0.27)/50% NMP).

FIG. 7: Plasma Octreotide Concentrations in Rabbits that Received a Subcutaneous Injection of a 90 mg ATRIGEL®/Octreotide Formulation; [symbol] Rabbit 1: ID# 3516; [symbol] Rabbit 2: ID# 3517; [symbol] Rabbit 3: ID# 3518; [symbol] Rabbit 4: ID# 3519; [symbol] Rabbit 5: ID# 3520; and [symbol] mean plasma level.

FIG. 8: Serum IGF-1 Levels in Rabbits that Received a Subcutaneous Injection of a 90 mg ATRIGEL®/Octreotide Formulation; [symbol] Rabbit 1: ID# 3516; [symbol] Rabbit 2: ID# 3517; [symbol] Rabbit 3: ID# 3518; [symbol] Rabbit 4: ID# 3519; [symbol] Rabbit 5: ID# 3520; and [symbol] Mean IGF-1 level.

FIG. 9: Correlation Between PK and PD in Rabbits that Received a Subcutaneous Injection of a 90 mg ATRIGEL®/Octreotide Formulation; [symbol] Rabbit mean Octreotide levels and [symbol] Rabbit mean IGF-1 levels.

FIG. 10: Release Profile of ATRIGEL®/Octreotide Formulations Following Subcutaneous Injection in Rats; [symbol] GpI: 15% OTCA in 50 Alkermes modified polymer process 85/15 PLGH (InV 0.25); [symbol] GpII: 15% OTCA in 50% 85/15 PLGH (InV 0.25) Alkermes+1.4% CH₂Cl₂; [symbol] GpIII: 15% OTCA in 50% 85/15 PLGH (InV 0.28), Alkermes; [symbol] GpIV: 15% OTCA in 50% 85/15 PLGH (InV 0.27), APT; and [symbol] GpV: 15% OTCA in 50% 85/15 PLGH (InV 0.25), Alkermes.

FIG. 11: Disposition of Subjects enrolled in the study.

FIG. 12: Mean (+SE) TSH Concentration-time Profiles Following Administration of Single s.c. Doses of ATRIGEL®/Octreotide 20 mg and Single i.m. Doses of Sandostatin LAR® 20 mg to Separate Groups of Subjects.

FIG. 13: Mean (+SE) Total T₄ Concentration-time Profiles Following Administration of Single s.c. Doses of ATRIGEL®/Octreotide 20 mg and Single i.m. Doses of Sandostatin LAR® 20 mg to Separate Groups of Subjects.

FIG. 14: Mean (+SE) Free T₄ Concentration-time Profiles Following Administration of Single s.c. Doses of ATRIGEL®/Octreotide 20 mg and Single i.m. Doses of Sandostatin LAR® 20 mg to Separate Groups of Subjects.

FIG. 15: Mean (+SD) Linear (0-48 hour) (FIG. 15(a)) and (O— Day 35) (FIG. 15(b)) Plasma Octreotide Concentration-time Profiles Following Administration of Single s.c. Doses of ATRIGEL®/Octreotide 20 mg and Single i.m. Doses Sandostatin LAR® 20 mg to Separate Groups of Subjects.

FIG. 16: Mean (+SD) Linear Serum IGF-1 Concentration-time Profiles (Day 0-14) (FIG. 16(a)) and (Day 14-70) (FIG. 16(b)) Following Administration of Single s.c. Doses of ATRIGEL®/Octreotide 20 mg and i.m. doses of Sandostatin LAR® 20 mg to Separate Groups of Subjects; Pharmacodynamic Data.

FIG. 17: Mean Linear (FIG. 17(a)) and Log-Linear (FIG. 17(b)) (+SD) Plasma Octreotide (0-48 hour) Profiles following Administration of a Single s.c. Dose of ATRIGEL®/Octreotide 20 mg and i.m. Dose of Sandostatin LAR® 20 mg; Pharmacodynamic Data.

FIG. 18: Mean Linear (FIG. 18(a)) and Log-Linear (FIG. 18(b)) (+SD) Plasma Octreotide Profiles following Administration of a Single s.c. Dose of ATRIGEL®/Octreotide 20 mg and i.m. Dose of Sandostatin LAR® 20 mg.

FIG. 19: Weight Distribution of ACF05-049 Octreotide ATRIGEL® SC injections.

FIG. 20: Extended Release of Octreotide ATRIGEL® from SC implants.

FIG. 21: Results from ACF05-036 and BTC(NP050812): Release of Octreotide ATRIGEL® from IVT and ST implants.

### DEFINITIONS

The words and phrases presented in this patent application have their ordinary meanings to one of skill in the art unless otherwise indicated. Such ordinary meanings can be obtained by reference to their use in the art and by reference to general and scientific dictionaries such as Webster's New World Dictionary, Simon & Schuster, publishers, New York, N.Y., 1995; The American Heritage Dictionary of the English Language, Houghton Mifflin, Boston Mass., 1981; Hawley's Condensed Chemical Dictionary 14^{th} edition, I. Sax, editor, Wiley Europe, 2002.

The following explanations of certain terms are meant to be illustrative rather than exhaustive. These terms have their ordinary meanings given by usage in the art and in addition include the following explanations.

The term “and/or” means any one of the items, any combination of the items, or all of the items with which this term is associated.

As used herein, the singular forms “a,” “an,” and “the” include plural reference unless the context clearly dictates otherwise. Thus, for example, a reference to “a formulation” includes a plurality of such formulations, so that a formulation of compound X includes formulations of compound X.

The term “amino acid,” means the residues of the natural amino acids (e.g. Ala, Arg, Asn, Asp, Cys, Glu, Gln, Gly, His, Hyl, Hyp, Ile, Leu, Lys, Met, Phe, Pro, Ser, Thr, Trp, Tyr, and Val) in D or L form, as well as unnatural amino acids (e.g. phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, ornithine, citruline, α-methyl-alanine, para-benzoylphenylalanine, phenylglycine, propargylglycine, sarcosine, and tert-butylglycine). The term also comprises natural and unnatural amino acids bearing a conventional amino protecting group (e.g. acetyl or benzyloxycarbonyl), as well as natural and unnatural amino acids protected at the carboxy terminus (e.g. as a (C₁-C₆) alkyl, phenyl or benzyl ester or amide; or as an α-methylbenzyl amide). Other suitable amino and carboxy protecting groups are known to those skilled in the art (See for example, Greene, T. W.; Wutz, P. G. M. “Protecting Groups In Organic Synthesis” second edition, 1991, New York, John Wiley & sons, Inc., and references cited therein).

The term “biocompatible” means that the material, substance, compound, molecule, polymer or system to which it applies will not cause severe toxicity, severe adverse biological reaction, or lethality in an animal to which it is administered at reasonable doses and rates.

The term “biodegradable” means that the material, substance, compound, molecule, polymer or system is cleaved, oxidized, hydrolyzed or otherwise broken down by hydrolytic, enzymatic or another mammalian biological process for metabolism to chemical units that can be assimilated or eliminated by the mammalian body.

The term “bioerodable” means that the material, substance, compound, molecule, polymer or system is biodegraded or mechanically removed by a mammalian biological process so that new surface is exposed.

As used herein, the term “flowable” refers to the ability of the “flowable” composition to be transported under pressure into the body of a patient. For example, the flowable composition can have a low viscosity like water, and be injected with the use of a syringe, beneath the skin of a patient. The flowable composition can alternatively have a high viscosity as in a gel and can be placed into a patient through a high pressure transport device such as a high pressure syringe, cannula, needle and the like. The ability of the composition to be injected into a patient will typically depend upon the viscosity of the composition. The composition will therefore have a suitable viscosity ranging from low like water to high like a gel, such that the composition can be forced through the transport device (e.g., syringe) into the body of a patient.

As used herein, a “gel” is a substance having a gelatinous, jelly-like, or colloidal properties. Concise Chemical and Technical Dictionary, 4th Enlarged Ed., Chemical Publishing Co., Inc., p. 567, NY, N.Y. (1986).

The term “heteroaromatic” refers to any aromatic compound or moiety containing carbon and one or more nitrogen and/or oxygen and/or sulfur atoms in the nucleus of the heteroaromatic structure. A heteroaromatic compound exhibits aromaticity such as that displayed by a pyridine, pyrimidine, pyrazine, indole thiazole, pyrrole, oxazole or similar compounds.

The term “heterocyclic” refers to any cyclic organic compound containing one or more nitrogen and/or oxygen and/or sulfur atoms in its cyclic structure. A heterocyclic compound may be saturated or unsaturated but is not aromatic.

As used herein, a “liquid” is a substance that undergoes continuous deformation under a shearing stress. Concise Chemical and Technical Dictionary, 4th Enlarged Ed., Chemical Publishing Co., Inc., p. 707, NY, N.Y. (1986).

The term “octreotide” is described in the following octreotide section, page 39.

The term “peptide” describes a sequence of 2 to about 50 amino acids (e.g. as defined hereinabove) or peptidyl residues. The sequence may be linear or cyclic. For example, a cyclic peptide can be prepared or may result from the formation of disulfide bridges between two cysteine residues in a sequence. Preferably a peptide comprises 3 to 30, or 5 to 20 amino acids. Peptide derivatives can be prepared as disclosed in U.S. Pat. Nos. 4,612,302; 4,853,371; and 4,684,620, or as described in the Examples herein below. Peptide sequences specifically recited herein are written with the amino terminus on the left and the carboxy terminus on the right.

The term “polymer” means a molecule of one or more repeating monomeric residue units covalently bonded together by one or more repeating chemical functional groups. The term includes all polymeric forms such as linear, branched, star, random, block, graft and the like. It includes homopolymers formed from a single monomer, copolymer formed from two or more monomers, terpolymers formed from three or more polymers and polymers formed from more than three monomers. Differing forms of a polymer may also have more than one repeating, covalently bonded functional group.

The term polyester refers to polymers containing monomeric repeats, at least in part, of the linking group: —OC(═O)— or —C(═O)O—.

The term polyanhydride refers to polymers containing monomeric repeats, at least in part, of the linking group —C(═O)—O—C(═O)—.

The term polycarbonate refers to polymers containing monomeric repeats, at least in part, of the linking group —OC(═O)O—.

The term polyurethane refers to polymers containing monomeric repeats, at least in part, of the linking group —NHC(═O)O—.

The term polyurea refers to polymers containing monomeric repeats, at least in part, of the linking group —NHC(═O)NH—.

The term polyamide refers to polymers containing monomeric repeats, at least in part, of the linking group —C(═O)NH—.

The term polyether refers to polymers containing monomeric repeats, at least in part, of the linking group —O—.

The term polyacetal refers to polymers containing monomeric repeats, at least in part, of the linking group —CHR—O—CHR—.

The term polyketal refers to polymers containing monomeric repeats, at least in part, of the linking group —CR₂—O—CR₂—.

The term “saccharide” refers to any sugar or other carbohydrate, especially a simple sugar or carbohydrate. Saccharides are an essential structural component of living cells and source of energy for animals. The term includes simple sugars with small molecules as well as macromolecular substances. Saccharides are classified according to the number of monosaccharide groups they contain.

The term “skin” and the term “core” of a skin and core matrix mean that a cross section of the matrix will present a discernable delineation between an outer surface and the inner portion of the matrix. The outer surface is the skin and the inner portion is the core.

The term “thermoplastic” as applied to a polymer means that the polymer repeatedly will melt upon heating and will solidify upon cooling. It signifies that no or only a slight degree of cross-linking between polymer molecules is present. It is to be contrasted with the term “thermoset” which indicates that the polymer will set or substantially cross-link upon heating or upon application of a similar reactive process and will then no longer undergo melt-solidification cycles upon heating and cooling.

### DESCRIPTION OF THE INVENTION

The present invention is directed to an octreotide sustained release delivery system. The sustained release delivery system includes a flowable composition of the invention and a gel or solid implant of the invention. The delivery system provides an in situ sustained release of octreotide. The flowable composition of the invention accomplishes the sustained release through its use to produce the implant of the invention. The implant has a low implant volume and provides a long term delivery of octreotide. The flowable composition enables subcutaneous formation of the implant in situ and causes little or no tissue necrosis. The in situ implant of the invention exhibits surprising results relative to the sustained release Sandostatin LAR® implant in that the implant of the invention delivers higher and longer lasting blood levels of the octreotide compared with the Sandostatin LAR® implant. It also exhibits a surprisingly low tissue irritation relative to Sandostatin LAR® implant.

The flowable composition of the invention is a combination of a biodegradable, at least substantially water-insoluble thermoplastic polymer, a biocompatible polar aprotic organic liquid and octreotide. The polar, aprotic organic liquid has a solubility in body fluid ranging from practically insoluble to completely soluble in all proportions. Preferably, the thermoplastic polymer is a thermoplastic polyester of one or more hydroxycarboxylic acids or one or more diols and dicarboxylic acids. Especially preferably, the thermoplastic polymer is a polyester of one or more hydroxylcarboxyl dimers such as lactide, glycolide, dicaprolactone and the like.

Specific and preferred biodegradable thermoplastic polymers and polar aprotic solvents; concentrations of thermoplastic polymers, polar aprotic organic liquids, octreotide, and molecular weights of the thermoplastic polymer; and weight or mole ranges of components of the solid implant described herein are exemplary. They do not exclude other biodegradable thermoplastic polymers and polar aprotic organic liquids; other concentrations of thermoplastic polymers, polar aprotic liquids, octreotide, or molecular weights of the thermoplastic polymer; and components within the solid implant.

The present invention is directed to a flowable composition suitable for use in providing a controlled sustained release implant, a method for forming the flowable composition, a method for using the flowable composition, the biodegradable sustained release solid or gel implant that is formed from the flowable composition, a method of forming the biodegradable implant in situ, a method for treating disease through use of the biodegradable implant and a kit that includes the flowable composition. The flowable composition may preferably be used to provide a biodegradable or bioerodible microporous in situ formed implant in animals.

The flowable composition is composed of a biodegradable thermoplastic polymer in combination with a biocompatible polar aprotic organic liquid and octreotide. The biodegradable thermoplastic polymer is substantially insoluble in aqueous medium and/or in body fluid, biocompatible, and biodegradable and/or bioerodible within the body of a patient. The flowable composition may be administered as a liquid or gel to tissue and forms an implant in situ. Alternatively, the implant may be formed ex vivo by combining the flowable composition with an aqueous medium. In this embodiment, the preformed implant may be surgically administered to the patient. In either embodiment, the thermoplastic polymer coagulates or solidifies to form the solid or gel implant upon the dissipation, dispersement or leaching of the organic liquid from the flowable composition when the flowable composition contacts a body fluid, an aqueous medium or water. The coagulation or solidification entangles and entraps the other components of the flowable composition such as octreotide, excipients, organic substances and the like so that they become dispersed within the gelled or solidified implant matrix. The flowable composition is biocompatible and the polymer matrix of the implant does not cause substantial tissue irritation or necrosis at the implant site. The implant delivers a sustained level of octreotide to the patient. Preferably, the flowable composition can be a liquid or a gel, suitable for injection in a patient (e.g., human).

The present invention surprisingly improves the bioavailability of a sustained release formulation of octreotide. According to the invention, the sustained release of octreotide has the ability to inhibit any abnormal cellular proliferation, which includes neovascularization, fibrosis, lymphoid proliferation, acromegaly and/or neoplastic growth such as carcinoid syndrome, occurring in any tissue, but particularly in ocular tissues. In the case of ocular tissues, maximal efficacy enables relatively high bioavailability of octreotide, because: (1) the blood-retinal barrier limits penetration into the ocular tissues; and (2) activation of somatostatin receptors in retinochoroidal tissues may require higher doses, and more sustained levels of octreotide.

In addition, the present invention provides: (a) relatively low volume injections; (b) improved local tissue tolerance at the injection site; (c) an opportunity to use a subcutaneous, or an intraocular, injection rather than an intramuscular injection; and (d) less frequent injections compared to other products.

The basis for the large differences in bioavailability and pharmacokinetics of the invention, compared with the Sandostatin LARS product, is not completely understood. However, it can be noted that the Sandostatin LAR® product is injected intramuscularly and it elicits a severe tissue reaction characterized by myonecrosis and intense acute inflammation. Gross and microscopic examination of intramuscular injection sites taken from a variety of animal species reveals extensive neutrophilic infiltration surrounding the Sandostatin LAR® product depots. A review of the summary basis of approval for the Sandostatin LAR® product does not mention this phenomenon. However, in multiple experiments conducted in rats, rabbits and dogs, these changes have been observed in every sample examined. In addition, oncologists and endocrinologists who chronically administer IM injections of the Sandostatin LAR® product to patients, have observed that this product produces severe tissue reactions leading to chronic scarring in the gluteal muscle tissues. Thus, the data indicate that chronic administration of the Sandostatin LAR® product to produce a Sandostatin® LAR depot is associated with adverse injection site reactions, which are not desirable in patients, and is especially not desirable in patients with diabetes or in elderly patients suffering from adult macular degeneration (AMD).

The severe tissue reaction surrounding the Sandostatin® depot not only produces pain and scarring, it may also contribute to the poor pharmacokinetics, which include a 7-10 day lag phase and a very low bioavailability. By comparison, the octreotide sustained release delivery system of the invention may be injected into the subcutaneous tissue. At the same dose of octreotide, experiments conducted in animals and humans have repeatedly indicated that the flowable composition of the invention provides much higher bioavailability as compared to the Sandostatin LAR® product, causes no tissue reaction and has no lag phase.

According to the present invention, the octreotide sustained release delivery system provides several advantages that increase the efficacy, safety, and convenience of octreotide used to treat any somatostatin-responsive disease or medical condition. This includes non-ocular and ocular diseases. The invention is particularly useful for the treatment of proliferative ocular diseases, and most particularly, for the treatment of neovascular diseases of the eye. Examples of such diseases include, but are not limited to, retinal or choroidal neovascularizaton, which occur in diabetic retinopathy and age-related macular degeneration, respectively.

By comparison to formulations derived from other sustained release drug delivery technologies, the octreotide sustained release delivery system will provide: (a) superior release kinetics with minimal burst; (b) increased duration of drug release with less frequent injections; (c) markedly improved bioavailability; (d) improved local tissue tolerance due to a small injection volume, and (e) the ability to use of a subcutaneous injection rather than intramuscular injection. Taken together, these features make a highly beneficial octreotide sustained release delivery system.

### Biodegradable Thermoplastic Polymer

The flowable composition of the invention is produced by combining a solid, biodegradable thermoplastic polymer and octreotide and a biocompatible polar aprotic organic liquid. The flowable composition can be administered by a syringe and needle to a patient in need of treatment. Any suitable biodegradable thermoplastic polymer can be employed, provided that the biodegradable thermoplastic polymer is at least substantially insoluble in body fluid.

The biocompatible, biodegradable, thermoplastic polymer used according to the invention can be made from a variety of monomers which form polymer chains or monomeric units joined together by linking groups. The thermoplastic polymer is composed of a polymer chain or backbone containing monomeric units joined by such linking groups as ester, amide, urethane, anhydride, carbonate, urea, esteramide, acetal, ketal, and orthocarbonate groups as well as any other organic functional group that can be hydrolyzed by enzymatic or hydrolytic reaction (i.e., is biodegradable by this hydrolytic action). The thermoplastic polymer is usually formed by reaction of starting monomers containing the reactant groups that will form the backbone linking groups. For example, alcohols and carboxylic acids will form ester linking groups. Isocyanates and amines or alcohols will respectively form urea or urethane linking groups.

Any aliphatic, aromatic or arylalkyl starting monomer having the specified functional groups can be used according to the invention to make the thermoplastic polymers of the invention, provided that the polymers and their degradation products are biocompatible. The monomer or monomers used in forming the thermoplastic polymer may be of a single or multiple identity. The resultant thermoplastic polymer will be a homopolymer formed from one monomer, or one set of monomers such as when a diol and diacid are used, or a copolymer, terpolymer, or multi-polymer formed from two or more, or three or more, or more than three monomers or sets of monomers. The biocompatiblity specifications of such starting monomers are known in the art.

The thermoplastic polymers useful according to the invention are substantially insoluble in aqueous media and body fluids, preferably essentially completely insoluble in such media and fluids. They are also capable of dissolving or dispersing in selected organic liquids having a water solubility ranging from completely soluble in all proportions to water insoluble. The thermoplastic polymers also are biocompatible.

When used in the flowable composition of the invention, the thermoplastic polymer in combination with the organic liquid provides a viscosity of the flowable composition that varies from low viscosity, similar to that of water, to a high viscosity, similar to that of a paste, depending on the molecular weight and concentration of the thermoplastic polymer. Typically, the polymeric composition includes about 10 wt. % to about 95 wt. %, more preferably about 20 wt. % to about 70 wt. %, most preferably about 30 wt. % to about 65 wt. %, of a thermoplastic polymer.

According to the present invention, the biodegradable, biocompatible thermoplastic polymer can be a linear polymer, it can be a branched polymer, or it can be a combination thereof. Any option is available according to the present invention. To provide a branched thermoplastic polymer, some fraction of one of the starting monomers may be at least trifunctional, and preferably multifunctional. This multifunctional character provides at least some branching of the resulting polymer chain. For example, when the polymer chosen contains ester linking groups along its polymer backbone, the starting monomers normally will be hydroxycarboxylic acids, cyclic dimers of hydroxycarboxylic acids, cyclic trimers of hydroxycarboxylic acids, diols or dicarboxylic acids. Thus, to provide a branched thermoplastic polymer, some fraction of a starting monomer that is at least multifunctional, such as a triol or a tricarboxylic acid is included within the combination of monomers being polymerized to form the thermoplastic polymer used according to the invention. In addition, the polymers of the present invention may incorporate more than one multifunctional unit per polymer molecule, and typically many multifunctional units depending on the stoichiometry of the polymerization reaction. The polymers of the present invention may also optionally incorporate at least one multifunctional unit per polymer molecule. A so-called star or branched polymer is formed when one multifunctional unit is incorporated in a polymer molecule.

According to the invention, the preferred thermoplastic polyester may be formed from such monomers as hydroxycarboxylic acids or dimers therefor. Alternatively, a thermoplastic polyester may be formed from a dicarboxylic acid and a diol. A branching monomer such as a dihydroxycarboxylic acid would be included with the first kind of starting monomer, or a triol and/or a tricarboxylic acid would be included with the second kind of starting monomer if a branched polyester were desired. Similarly, a triol, tetraol, pentaol, or hexaol such as sorbitol or glucose can be included with the first kind of starting monomer if a branched or star polyester were desired. The same rationale would apply to polyamides. A triamine and/or triacid would be included with starting monomers of a diamine and dicarboxylic acid. An amino dicarboxylic acid, diamino carboxylic acid or a triamine would be included with the second kind of starting monomer, amino acid. Any aliphatic, aromatic or arylalkyl starting monomer having the specified functional groups can be used to make the branched thermoplastic polymers of the invention, provided that the polymers and their degradation products are biocompatible. The biocompatiblity specifications of such starting monomers are known in the art.

The monomers used to make the biocompatible thermoplastic polymers of the present invention will produce polymers or copolymers that are thermoplastic, biocompatible and biodegradable. Examples of thermoplastic, biocompatible, biodegradable polymers suitable for use as the biocompatible thermoplastic branched polymers of the present invention include polyesters, polylactides, polyglycolides, polycaprolactones, polyaiihydrides, polyamides, polyurethanes, polyesteramides, polydioxanones, polyacetals, polyketals, polycarbonates, polyorthocarbonates, polyorthoesters, polyphosphoesters, polyphosphazenes, polyhydroxybutyrates, polyhydroxyvalerates, polyalkylene oxalates, polyalkylene succinates, poly(malic acid), poly(amino acids), and copolymers, terpolymers, or combinations or mixtures of the above materials. Suitable examples of such biocompatible, biodegradable, thermoplastic polymers are disclosed, e.g., in U.S. Pat. Nos. 4,938,763; 5,278,201; 5,324,519; 5,702,716; 5,744,153; 5,990,194; 6,461,631 and 6,565,874.

The polymer composition of the invention can also include polymer blends of the polymers of the present invention with other biocompatible polymers, so long as they do not interfere undesirably with the biodegradable characteristics of the composition. Blends of the polymer of the invention with such other polymers may offer even greater flexibility in designing the precise release profile desired for targeted drug delivery or the precise rate of biodegradability desired for implants such as ocular implants.

The preferred biocompatible thermoplastic polymers or copolymers of the present invention are those which have a lower degree of crystallization and are more hydrophobic. These polymers and copolymers are more soluble in the biocompatible organic liquids than highly crystalline polymers such as polyglycolide, which has a high degree of hydrogen-bonding. Preferred materials with the desired solubility parameters are polylactides, polycaprolactones, and copolymers of these with glycolide so as to provide more amorphous regions to enhance solubility. Generally, the biocompatible, biodegradable thermoplastic polymer is substantially soluble in the organic liquid so that solutions, dispersions or mixtures up to 50-60 wt % solids can be made. Preferably, the polymers used according to the invention are essentially completely soluble in the organic liquid so that solutions, dispersions or mixtures up to 85-98 wt % solids can be made. The polymers also are at least substantially insoluble in water so that less than 0.1 g of polymer per mL of water will dissolve or disperse in water. Preferably, the polymers used according to the invention are essentially completely insoluble in water so that less than 0.001 g of polymer per mL of water will dissolve or disperse in water. At this preferred level, the flowable composition with a completely water miscible organic liquid will almost immediately transform to the solid implant.

Optionally, the delivery system may also contain a combination of a non-polymeric material and an amount of a thermoplastic polymer. The combination of non-polymeric material and thermoplastic polymer may be adjusted and designed to provide a more coherent octreotide sustained release delivery system.

Non-polymeric materials useful in the present invention are those that are biocompatible, substantially insoluble in water and body fluids, and biodegradable and/or bioerodible within the body of an animal. The non-polymeric material is capable of being at least partially solubilized in an organic liquid. In the flowable composition of the invention containing some organic liquid or other additive, the non-polymeric materials are also capable of coagulating or solidifying to form a solid or gel implant upon the dissipation, dispersement or leaching of the organic liquid component from the flowable composition upon contact of the flowable composition with a body fluid. The matrix of all embodiments of the implant including a non-polymeric material will have a consistency ranging from gelatinous to impressionable and moldable, to a hard, dense solid.

Non-polymeric materials that can be used in the delivery system generally include any having the foregoing characteristics. Examples of useful non-polymeric materials include sterols such as cholesterol, stigmasterol, beta-sistosterol, and estradiol; cholestery esters such as cholesteryl stearate, C18-C36 mono-,di-, and tricylglycerides such as glyceryl monooleate, glyceryl monolinoleate, glyceryl monolaurate, glyceryl monodocosanoate, glyceryl monomyristate, glyceryl monodicenoate, glyceryl dipalmitate, glyceryl didocosanoate, glyceryl dimyristate, glyceryl tridocosanoate, glyceryl trimyristate, glyceryl tridecenoate, glyceryl tristearate and mixtures thereof; sucrose fatty acid esters such as sucrose distearate and sucrose palmitate; sorbitan fatty acid esters such as sorbitan monostearate, sorbitan monopalmitate, and sorbitan tristearate; C16-C18 fatty alcohols such as cetyl alcohol, myristyl alcohol, stearyl alcohol, and cetostearyl alcohol; esters of fatty alcohols and fatty acids such as cetyl palmitate and cetearyl palmitate; anhydrides of fatty acids such as stearic anhydride; phospholipids including phosphatidylcholine (lecithin), phosphatidylserine, phosphatidylethanolamine, phosphatidylinositol, and lysoderivatives thereof; sphingosine and derivatives thereof; spingomyelins such as stearyl, palmitoyl, and tricosanyl sphingomyelins; ceramides such as stearyl and palmitoyl ceramides; glycosphingolipids; lanolin and lanolin alcohols; and combinations and mixtures thereof. Preferred non-polymeric materials include cholesterol, glyceryl monostearate, glyceryl tristearate, stearic acid, stearic anhydride, glyceryl monooleate, glyeryl monolinoleate, and acetylated monoglyerides.

The polymeric and non-polymeric materials may be selected and/or combined to control the rate of biodegradation, bioerosion and/or bioabsorption within the implant site. Generally, the implant matrix will breakdown over a period from about 1 week to about 12 months, preferably over a period of about 1 week to about 4 months.

### Thermoplastic Polymer Molecular Weight

The molecular weight of the polymer used in the present invention can affect the rate of octreotide release from the implant. Under these conditions, as the molecular weight of the polymer increases, the rate of octreotide release from the system decreases. This phenomenon can be advantageously used in the formulation of systems for the controlled release of octreotide. For relatively quick release of octreotide, low molecular weight polymers can be chosen to provide the desired release rate. For release of a octreotide over a relatively long period of time, a higher polymer molecular weight can be chosen. Accordingly, an octreotide sustained release delivery system can be produced with an optimum polymer molecular weight range for the release of octreotide over a selected length of time.

The molecular weight of a polymer can be varied by any of a variety of methods. The choice of method is typically determined by the type of polymer composition. For example, if a thermoplastic polyester is used that is biodegradable by hydrolysis, the molecular weight can be varied by controlled hydrolysis, such as in a steam autoclave. Typically, the degree of polymerization can be controlled, for example, by varying the number and type of reactive groups and the reaction times.

The control of molecular weight and/or inherent viscosity of the thermoplastic polymer is a factor involved in the formation and performance of the implant. In general, thermoplastic polymers with higher molecular weight and higher inherent viscosity will provide an implant with a slower degradation rate and therefore a longer duration. Changes and fluxuations of the molecular weight of the thermoplastic polymer following the compounding of the delivery system will result in the formation of an implant that shows a degradation rate and duration substantially different from the degradation rate and duration desired or predicted.

The thermoplastic polymers useful according to the invention may have average molecular weights ranging from about 1 kiloDalton (kD) to about 1,000 kD, preferably from about 2 kD to about 500 kD, more preferably from abut 5 kD to about 200 kD, and most preferably from about 5 kD to about 100 kD. The molecular weight may also be indicated by the inherent viscosity (abbreviated as “I.V.”, units are in deciliters/gram). Generally, the inherent viscosity of the thermoplastic polymer is a measure of its molecular weight and degradation time (e.g., a thermoplastic polymer with a high inherent viscosity has a higher molecular weight and longer degradation time). Preferably, the thermoplastic polymer has a molecular weight, as shown by the inherent viscosity, from about 0.05 dL/g to about 2.0 dL/g (as measured in chloroform), more preferably from about 0.10 dL/g to about 1.5 dL/g.

### Characteristics of Preferred Polyester

The preferred thermoplastic biodegradable polymer of the flowable composition of the invention is a polyester. Generally, the polyester may be composed of units of one or more hydroxycarboxylic acid residues wherein the distribution of differing units may be random, block, paired or sequential. Alternatively, the polyester may be composed of units of one or more diols and one or more dicarboxylic acids. The distribution will depend upon the starting materials used to synthesize the polyester and upon the process for synthesis. An example of a polyester composed of differing paired units distributed in block or sequential fashion is a poly(lactide-co-glycolide). An example of a polyester composed of differing unpaired units distributed in random fashion is poly (lactic acid-co-glycolic acid). Other examples of suitable biodegradable thermoplastic polyesters include polylactides, polyglycolides, polycaprolactones, copolymers thereof, terpolymers thereof, and any combinations thereof. Preferably, the suitable biodegradable thermoplastic polyester is a polylactide, a polyglycolide, a copolymer thereof, a terpolymer thereof, or a combination thereof.

The terminal groups of the poly(DL-lactide-co-glycolide) can either be hydroxyl, carboxyl, or ester depending upon the method of polymerization. Polycondensation of lactic or glycolic acid will provide a polymer with terminal hydroxyl and carboxyl groups. Ring-opening polymerization of the cyclic lactide or glycolide monomers with water, lactic acid, or glycolic acid will provide polymers with these same terminal groups. However, ring-opening of the cyclic monomers with a monofunctional alcohol such as methanol, ethanol, or 1-dodecanol will provide a polymer with one hydroxyl group and one ester terminal group. Ring-opening polymerization of the cyclic monomers with a polyol such as glucose, 1,6-hexanediol or polyethylene glycol will provide a polymer with only hydroxyl terminal groups. Such a polymerization of dimers of hydroxylcarboxylic acids and a polyol is a chain extension of the polymer. The polyol acts as a central condensation point with the polymer chain growing from the hydroxyl groups incorporated as ester moieties of the polymer. The polyol may be a diol, triol, tetraol, pentaol or hexaol of 2 to 30 carbons in length. Examples include saccharides, reduced saccharides such as sorbitol, diols such as hexane-1,6-diol, triols such as glycerol or reduced fatty acids, and similar polyols. Generally, the polyesters copolymerized with alcohols or polyols will provide longer duration implants.

The type, molecular weight, and amount of the preferred biodegradable thermoplastic polyester present in the flowable composition will typically depend upon the desired properties of the controlled sustained release implant. For example, the type, molecular weight, and amount of biodegradable thermoplastic polyester can influence the length of time in which the octreotide is released from the controlled sustained release implant. Specifically, in one embodiment of the present invention, the composition can be used to formulate a one month sustained release delivery system of octreotide. In such an embodiment, the biodegradable thermoplastic polyester can be a 50/50, 55/45, 75/25, 85/15, 90/10, or 95/5 poly (DL-lactide-co-glycolide) having a carboxy terminal group, preferably a 50/50 poly (DL-lactide-co-glycolide) having a carboxy terminal group; can be present in about 20 wt. % to about 70 wt. % of the composition; and can have an average molecular weight of about 15,000 to about 45,000, about 23,000 to about 45,000, or about 20,000 to about 40,000.

In another embodiment of the present invention, the flowable composition can be formulated to provide a three month sustained release delivery system of octreotide. In such an embodiment, the biodegradable thermoplastic polyester can be a 50/50, 55/45, 75/25, 85/15, 90/10, or 95/5 poly (DL-lactide-co-glycolide) without a carboxy terminal group; preferably be a 75/25 poly (DL-lactide-co-glycolide) without a carboxy terminal group; can be present in about 20 wt. % to about 70 wt. % of the composition; and can have an average molecular weight of about 20,000 to about 40,000, or about 15,000 to about 25,000; or can be an 85/15 poly (DL-lactide-co-glycolide) containing a 1,6-hexane diol chain extender, at a weight percentage of about 20 wt. % to about 70 wt. % of the flowable composition and at an average molecular weight of about 15,000 to about 30,000. Any polyester that has a terminal carboxyl group can optionally be extended with a diol moiety.

### Polar Aprotic Organic Solvent

Organic liquids suitable for use in the flowable composition of the invention are biocompatible and display a range of solubilities in aqueous medium, body fluid, or water. That range includes complete insolubility at all concentrations upon initial contact, to complete solubility at all concentrations upon initial contact between the organic liquid and the aqueous medium, body fluid or water.

While the solubility or insolubility of the organic liquid in water can be used as a solubility guide according to the invention, its water solubility or insolubility in body fluid typically will vary from its solubility or insolubility in water. Relative to water, body fluid contains physiologic salts, lipids, proteins and the like, and will have a differing solvating ability for organic liquids. This phenomenon is similar to the classic “salting out” characteristic displayed by saline relative to water. Body fluid displays similar variability relative to water but in contrast to a “salting out” factor, body fluid typically has a higher solvating ability for most organic liquids than does water. This higher ability is due in part to the greater lipophilic character of body fluid relative to water, and also in part to the dynamic character of body fluid. In a living organism, body fluid is not static but rather moves throughout the organism. In addition, body fluid is purged or cleansed by tissues of the organism so that body fluid contents are removed. As a result, body fluid in living tissue will remove, solvate or dissipate organic liquids that are utterly insoluble in water.

Pursuant to the foregoing understanding of the solubility differences among water, aqueous media and body fluid, the organic liquid used in the present invention may be completely insoluble to completely soluble in water when the two are initially combined. Preferably the organic liquid is at least slightly soluble, more preferably moderately soluble, especially more preferably highly soluble, and most preferably soluble at all concentrations in water. The corresponding solubilities of the organic liquids in aqueous media and body fluid will tend to track the trends indicated by the water solubilities. In body fluid, the solubilities of the organic liquids will tend to be higher than those in water.

When an organic liquid that is insoluble to only slightly soluble in body fluid is used in any of the embodiments of the sustained release delivery system, it will allow water to permeate into the implanted delivery system over a period of time ranging from seconds to weeks or months. This process may decrease or increase the delivery rate of the octreotide and in the case of the flowable composition, it will affect the rate of coagulation or solidification. When an organic liquid that is moderately soluble to very soluble in body fluid is used in any of the embodiments of the delivery system, it will diffuse into body fluid over a period of minutes to days. The diffusion rate may decrease or increase the delivery rate of the octreotide. When highly soluble organic liquids are used, they will diffuse from the delivery system over a period of seconds to hours. Under some circumstances, this rapid diffusion is responsible at least in part for the so-called burst effect. The burst effect is a short-lived but rapid release of octreotide upon implantation of the delivery system followed by a long-lived, slow release of octreotide.

Organic liquids used in the delivery system of the present invention include aliphatic, aryl, and arylalkyl; linear, cyclic and branched organic compounds that are liquid or at least flowable at ambient and physiological temperature and contain such functional groups as alcohols, alkoxylated alcohols, ketones, ethers, polymeric ethers, amides, esters, carbonates, sulfoxides, sulfones, any other functional group that is compatible with living tissue, and any combination thereof. The organic liquid preferably is a polar aprotic or polar protic organic solvent. Preferably, the organic liquid has a molecular weight in the range of about 30 to about 1000.

Preferred biocompatible organic liquids that are at least slightly soluble in aqueous or body fluid include N-methyl-2-pyrrolidone, 2-pyrrolidone; C₁ to C₁₅ alcohols, diols, triols and tetraols such as ethanol, glycerine, propylene glycol, butanol; C₃ to C₁₅ alkyl ketones such as acetone, diethyl ketone and methyl ethyl ketone; C₃ to C₁₅ esters and alkyl esters of mono-, di-, and tricarboxylic acids such as 2-ethyoxyethyl acetate, ethyl acetate, methyl acetate, ethyl lactate, ethyl butyrate, diethyl malonate, diethyl glutonate, tributyl citrate, diethyl succinate, tributyrin, isopropyl myristate, dimethyl adipate, dimethyl succinate, dimethyl oxalate, dimethyl citrate, triethyl citrate, acetyl tributyl citrate, and glyceryl triacetate; C₁ to C₁₅ amides such as dimethylformamide, dimethylacetamide and caprolactam; C₃ to C₂₀ ethers such as tetrahydrofuran, or solketal; tweens, triacetin, decylmethylsulfoxide, dimethyl sulfoxide, oleic acid, 1-dodecylazacycloheptan-2-one, N-methyl-2-pyrrolidone, esters of carbonic acid and alkyl alcohols such as propylene carbonate, ethylene carbonate, and dimethyl carbonate; alkyl ketones such as acetone and methyl ethyl ketone; alcohols such as solketal, glycerol formal, and glycofurol; dialkylamides such as dimethylformamide, dimethylacetamide, dimethylsulfoxide, and dimethylsulfone; lactones such as epsilon-caprolactone and butyrolactone; cyclic alkyl amides such as caprolactam; triacetin and diacetin; aromatic amides such as N,N-dimethyl-m-toluamide, and mixtures and combinations thereof. Preferred solvents include N-methyl-2-pyrrolidone, 2-pyrrolidone, dimethylsulfoxide, ethyl lactate, propylene carbonate, solketal, triacetin, glycerol formal, isopropylidene glycol, and glycofurol.

Other preferred organic liquids are benzyl alcohol, benzyl benzoate, dipropylene glycol, tributyrin, ethyl oleate, glycerin, glycofaral, isopropyl myristate, isopropyl palmitate, oleic acid, polyethylene glycol, propylene carbonate, and triethyl citrate. The most preferred solvents are N-methyl-2-pyrrolidone, 2-pyrrolidone, dimethyl sulfoxide, triacetin, and propylene carbonate because of their solvating ability and their compatibility.

The type and amount of biocompatible organic liquid present in the flowable composition will typically depend on the desired properties of the controlled release implant as described in detail below. Preferably, the flowable composition includes about 0.001 wt % to about 90 wt %, more preferably about 5 wt % to about 70 wt %, most preferably 5 to 60 wt % of an organic liquid.

The solubility of the biodegradable thermoplastic polymers in the various organic liquids will differ depending upon their crystallinity, their hydrophilicity, hydrogen-bonding, and molecular weight. Lower molecular-weight polymers will normally dissolve more readily in the organic liquids than high-molecular-weight polymers. As a result, the concentration of a thermoplastic polymer dissolved in the various organic liquids will differ depending upon type of polymer and its molecular weight. Moreover, the higher molecular-weight thermoplastic polymers will tend to give higher solution viscosities than the low-molecular-weight materials.

When the organic liquid forms part of the flowable composition of the invention, it functions not only to enable easy, non-surgical placement of the sustained release delivery system into living tissue. It also facilitates transformation of the flowable composition to an in situ formed implant. Although it is not meant as a limitation of the invention, it is believed that the transformation of the flowable composition is the result of the dissipation of the organic liquid from the flowable composition into the surrounding body fluid and tissue and the infusion of body fluid from the surrounding tissue into the flowable composition. It is believed that during this transformation, the thermoplastic polymer and organic liquid within the flowable composition partition into regions rich and poor in polymer.

For the flowable composition of the invention, the concentration of the thermoplastic polymer in the organic liquid according to the invention will range from about 0.01 g per mL of organic liquid to a saturated concentration. Typically, the saturated concentration will be in the range of 80 to 95 wt % solids or 4 to almost 5 gm per mL of organic liquid, assuming that the organic liquid weighs approximately 1 gm per mL.

For polymers that tend to coagulate slowly, a solvent mixture can be used to increase the coagulation rate. In essence, one liquid component of the solvent mixture is a good solvent for the polymer, and the other liquid component of the solvent mixture is a poorer solvent or a non-solvent. The two liquids are mixed at a ratio such that the polymer is still soluble but precipitates with the slightest increase in the amount of non-solvent, such as water in a physiological environment. By necessity, the solvent system must be miscible with both the polymer and water. An example of such a binary solvent system is the use of N-methylpyrrolidone and ethanol. The addition of ethanol to the NMP/polymer solution increases its coagulation rate.

For the formed implant of the invention, the presence of the organic liquid can serve to provide the following properties: plasticization, moldability, flexibility, increased or decreased homogeneity, increased or decreased release rate for the bioactive agent, leaching, promotion or retardation of body fluid influx into the implant, patient comfort, compatibility of thermoplastic polymer and bioactive agent and the like. Generally the concentration of organic liquid in the formed implant may range from about 0.001 wt. % to as much as about 30 wt. %. Generally, the concentration will be less than an amount that would cause reversion of the formed implant into a flowable composition. Also, the organic liquid may preferentially be chosen so as to display less than substantial ability to dissolve the thermoplastic polymer.

The pliability of the implant can be substantially maintained throughout its life if additives such as the organic liquid are maintained in the implant. Such additives also can act as a plasticizer for the thermoplastic polymer and at least in part may remain in the implant. One such additive having these properties is an organic liquid of low water solubility to water insolubility. Such an organic liquid providing these pliability and plasticizing properties may be included in the delivery system as the sole organic liquid or may be included in addition to an organic liquid that is moderately to highly water soluble.

Organic liquids of low water solubility or water insolubility, such as those forming aqueous solutions of no more than 5% by weight in water, can function as a pliability, plasticizing component and in addition can act as the solvating component for the flowable composition embodiment of the invention.

Such organic liquids can act as plasticizers for the thermoplastic polymer. When the organic liquid has these properties, it is a member of a subgroup of organic liquids termed “plasticizer”. The plasticizer influences the pliablity and moldability of the implant composition such that it is rendered more comfortable to the patient when implanted. Moreover, the plasticizer has an effect upon the rate of sustained release of octreotide such that the rate can be increased or decreased according to the character of the plasticizer incorporated into the implant composition. In general, the organic liquid acting as a plasticizer is believed to facilitate molecular movement within the solid or gel thermoplastic matrix. The plasticizing capability enables polymer molecules of the matrix to move relative to each other so that pliability and easy moldability are provided. The plasticizing capability also enables easy movement of octreotide so that in some situations, the rate of sustained release is either positively or negatively affected.

### High Water Solubility Organic Liquids

A moderate to highly water soluble organic liquid can be generally used in the flowable composition of the invention, especially when pliability will not be an issue after formation of the implant. Use of the highly water soluble organic liquid will provide an implant having the physical characteristics of an implant made through direct insertion of the flowable composition.

Use of a moderate to highly water soluble organic liquid in flowable composition of the invention will facilitate intimate combination and mixture of the other components therein. It will promote solid or gel homogeneity and pliability of an ex vivo formed implant so that such an implant can be readily inserted into appropriate incisions or trocar placements in tissue.

Useful, highly water soluble organic liquids include, for example, substituted heterocyclic compounds such as N-methyl-2-pyrrolidone (NMP) and 2-pyrrolidone; C₂ to C₁₀ alkanoic acids such as acetic acid and lactic acid, esters of hydroxy acids such as methyl lactate, ethyl lactate, alkyl citrates and the like; monoesters of polycarboxylic acids such as monomethyl succinate acid, monomethyl citric acid and the like; ether alcohols such as glycofurol, glycerol formal, isopropylidene glycol, 2,2-dimethyl-1,3-dioxolone-4-methanol; Solketal; dialkylamides such as dimethylformamide and dimethylacetamide; dimethylsulfoxide (DMSO) and dimethylsulfone; lactones such as epsilon, caprolactone and butyrolactone; cyclic alkyl amides such as caprolactam; and mixtures and combinations thereof. Preferred organic liquids include N-methyl-2-pyrrolidone, 2-pyrrolidone, dimethylsulfoxide, ethyl lactate, glycofurol, glycerol formal, and isopropylidene glycol.

### Low Water Solubility Organic Liquids/Solvents

As described above, an organic liquid of low or no water solubility (hereinafter low/no liquid) may also be used in the sustained release delivery system. Preferably, a low/no liquid is used when it is desirable to have an implant that remains pliable, is to be extrudable is to have an extended release and the like. For example, the release rate of the biologically active agent can be affected under some circumstances through the use of a low/no liquid. Typically such circumstances involve retention of the organic liquid within the implant product and its function as a plasticizer or rate modifier.

Examples of low or nonsoluble organic liquids include esters of carbonic acid and aryl alcohols such as benzyl benzoate; C₄ to C₁₀ alkyl alcohols; C₁ to C₆ alkyl C₂ to C₆ alkanoates; esters of carbonic acid and alkyl alcohols such as propylene carbonate, ethylene carbonate and dimethyl carbonate, alkyl esters of mono-, di-, and tricarboxylic acids, such as 2-ethyoxyethyl acetate, ethyl acetate, methyl acetate, ethyl butyrate, diethyl malonate, diethyl glutonate, tributyl citrate, diethyl succinate, tributyrin, isopropyl myristate, dimethyl adipate, dimethyl succinate, dimethyl oxalate, dimethyl citrate, triethyl citrate, acetyl tributyl citrate and glyceryl triacetate; alkyl ketones such as methyl ethyl ketone; as well as other carbonyl, ether, carboxylic ester, amide and hydroxy containing liquid organic compounds having some solubility in water. Propylene carbonate, ethyl acetate, triethyl citrate, isopropyl myristate, and glyceryl triacetate are preferred because of biocompatitibility and pharmaceutical acceptance.

Additionally, mixtures of the foregoing high and low or no solubility organic liquids providing varying degrees of solubility for the matrix forming material can be used to alter the life time, rate of bioactive agent release and other characteristics of the implant. Examples include a combination of N-methylpyrrolidone and propylene carbonate, which provides a more hydrophobic solvent than N-methylpyrrolidone alone, and a combination of N-methylpyrrolidone and polyethylene glycol, which provides a more hydrophilic solvent than N-methylpyrrolidone alone.

The organic liquid for inclusion in the composition should be biocompatible. Biocompatible means that as the organic liquid disperses or diffuses from the composition, it does not result in substantial tissue irritation or necrosis surrounding the implant site.

### Organic Liquid for the Preferred Flowable Composition

For the preferred flowable composition incorporating a thermoplastic polyester, any suitable polar aprotic organic liquid can be employed, provided that the suitable polar aprotic solvent displays a body fluid solubility within a range of completely soluble in all proportions to only very slightly soluble. Suitable polar aprotic organic liquids are disclosed, e.g., in Aldrich Handbook of Fine Chemicals and Laboratory Equipment, Milwaukee, Wis. (2000); U.S. Pat. Nos. 5,324,519; 4,938,763; 5,702,716; 5,744,153; and 5,990,194. A suitable polar aprotic liquid should be able to diffuse over time into body fluid so that the flowable composition coagulates or solidifies. The diffusion may be rapid or slow. It is also preferred that the polar aprotic liquid for the biodegradable polymer be non-toxic and otherwise biocompatible.

The polar aprotic organic liquid is preferably biocompatible. Examples of suitable polar aprotic organic liquid include those having an amide group, an ester group, a carbonate group, a ketone, an ether, a sulfonyl group, or a combination thereof. Examples are mentioned above.

Preferably, the polar aprotic organic liquid can be N-methyl-2-pyrrolidone, 2-pyrrolidone, N,N-dimethylformamide, dimethyl sulfoxide, propylene carbonate, caprolactam, triacetin, or any combination thereof. More preferably, the polar aprotic organic solvent can be N-methyl-2-pyrrolidone.

The solubility of the biodegradable thermoplastic polyesters in the various polar aprotic liquids will differ depending upon their crystallinity, their hydrophilicity, hydrogen-bonding, and molecular weight. Thus, not all of the biodegradable thermoplastic polyesters will be soluble to the same extent in the same polar aprotic organic liquid, but each biodegradable thermoplastic polymer or copolymer should be soluble in its appropriate polar aprotic solvent. Lower molecular-weight polymers will normally dissolve more readily in the liquids than high-molecular-weight polymers. As a result, the concentration of a polymer dissolved in the various liquids will differ depending upon type of polymer and its molecular weight. Conversely, the higher molecular-weight polymers will normally tend to coagulate or solidify faster than the very low-molecular-weight polymers. Moreover the higher molecular-weight polymers will tend to give higher solution viscosities than the low-molecular-weight materials.

For example, low-molecular-weight polylactic acid formed by the condensation of lactic acid will dissolve in N-methyl-2-pyrrolidone(NMP) to give a 73% by weight solution which still flows easily through a 23-gauge syringe needle, whereas a higher molecular-weight poly(DL-lactide) (DL-PLA) formed by the additional polymerization of DL-lactide gives the same solution viscosity when dissolved in NMP at only 50% by weight. The higher molecular-weight polymer solution coagulates immediately when placed into water. The low-molecular-weight polymer solution, although more concentrated, tends to coagulate very slowly when placed into water.

It has also been found that solutions containing very high concentrations of high molecular weight polymers sometimes coagulate or solidify slower than more dilute solutions. It is believed that the high concentration of polymer impedes the diffusion of solvent from within the polymer matrix and consequently prevents the permeation of water into the matrix where it can precipitate the polymer chains. Thus, there is an optimum concentration at which the solvent can diffuse out of the polymer solution and water penetrates within to coagulate the polymer.

The concentration and species of the polar aprotic organic liquid for the preferred flowable composition of the invention incorporating a thermoplastic polyester will typically depend upon the desired properties of the controlled release implant. For example, the species and amount of biocompatible polar aprotic solvent can influence the length of time in which the octreotide is released from the controlled release implant. Specifically, in one embodiment of the present invention, the flowable composition can be used to formulate a one month delivery system of octreotide. In such an embodiment, the biocompatible polar aprotic solvent can preferably be N-methyl-2-pyrrolidone and can preferably present in about 30 wt. % to about 60 wt. % of the composition. Alternatively, in another embodiment of the present invention, the composition can be used to formulate a three month delivery system of octreotide. In such an embodiment, the biocompatible polar aprotic solvent can preferably be N-methyl-2-pyrrolidone and can preferably present in about 20 wt. % to about 60 wt. % of the composition.

### Octreotide

Octreotide is a known oligopeptide of the peptide sequence Phe-Cys-Phe-Trp-Lys-Thr-Cys. Octreotide typically includes a disulfide link between the cysteines, and the phenylalanine (Phe) and the tryptophan (Trp) are in the D configuration although their L configurations may also be included. The C-terminus cysteine may be terminated as a carboxyl or may be amidated with an organic amine such as an alkyl amine, a dialkyl amine, or a hydroxylalkyl amine. Preferably, the amidating group is 2-hydroxy-1-hydroxymethyl propyl amine. The C-terminus cysteine may also be amidated with an additional amino acid unit such as threonine (Thr), serine (Ser) or tyrosine (Thy) and the resulting C-terminus of the amidating amino acid may be carboxyl or amidated as described for the C-terminus cysteine. The preferred amidating amino acid group is threonine. The peptide sequence may also be glycosylated at the N-terminus. The glycosylation groups may be galactosyl, glucosyl, glucosyl-fructosyl as well as other disaccharidysyl glycosylation groups.

Octreotide may be administered in its ulmeutralized basic form owing to the basic side chains of the tryptophan and lysine units, or as a salt of an organic or inorganic acid. Examples include the octreotide salts wherein the gegenion (counter-ion) is acetate, propionate, tartrate, malonate, chloride, sulfate, bromide, and other pharmaceutically acceptable organic and inorganic acid gegenions. Preferred are organic acids with multiple carboxylic acid groups such as malonic acid, citric acid, itaconic acid, adipic acid and di-, tri- and tetra-carboxylic acids of four to 40 carbon atoms.

Octreotide is preferably lyophilized prior to use. Typically, the octreotide can be dissolved in an aqueous solution, sterile filtered and lyophilized in a syringe. In a separate process, the thermoplastic polymer/organic liquid solution can be filled into second syringe. The two syringes can then be coupled together and the contents can be drawn back and forth between the two syringes until the thermoplastic polymer, organic liquid and the octreotide are effectively mixed together, forming a flowable composition. The flowable composition can be drawn into one syringe. The two syringes can then be discolmected and a needle attached to the syringe containing the flowable composition. The flowable composition can then be injected through the needle into the body. The flowable composition can be formulated and administered to a patient as described in, e.g., U.S. Pat. Nos. 5,324,519; 4,938,763; 5,702,716; 5,744,153; and 5,990,194; or as described herein. Once administered, the organic liquid dissipates, the remaining polymer gels or solidifies, and a matrix structure is formed. The organic liquid will dissipate and the polymer will solidify or gel so as to entrap or encase the octreotide within the matrix.

The release of octreotide from the implant of the invention will follow the same general rules for release of a drug from a monolithic polymeric device. The release of octreotide can be affected by the size and shape of the implant, the loading of octreotide within the implant, the permeability factors involving the octreotide and the particular polymer, and the degradation of the polymer. Depending upon the amount of octreotide selected for delivery, the above parameters' can be adjusted by one skilled in the art of drug delivery to give the desired rate and duration of release.

The amount of octreotide incorporated into the sustained release delivery system of the invention depends upon the desired release profile, the concentration of octreotide required for a biological effect, and the length of time that the octreotide has to be released for treatment. There is no upper limit on the amount of octreotide incorporated into the sustained release delivery system except for that of an acceptable solution or dispersion viscosity for injection through a syringe needle. The lower limit of octreotide incorporated into the sustained release delivery system is dependent upon the activity of the octreotide and the length of time needed for treatment. Specifically, in one embodiment of the present invention, the sustained release delivery system can be formulated to provide a one month release of octreotide. In such an embodiment, the octreotide can preferably be present in about 1 wt. % to about 20 wt. %, preferably about 8 wt. % to about 15 wt. % of the composition. Alternatively, in another embodiment of the present invention, the sustained release delivery system can be formulated to provide a three month delivery of octreotide. In such an embodiment, the octreotide can preferably be present in about 1 wt. % to about 20 wt. %, perferrably about 8 wt. % to about 15 wt. % of the composition. The gel or solid implant formed from the flowable composition will release the octreotide contained within its matrix at a controlled rate until the implant is effectively depleted of octreotide.

### Adjuvants and Carriers

The sustained release delivery system may include a release rate modifier to alter the sustained release rate of octreotide from the implant matrix. The use of a release rate modifier may either decrease or increase the release of octreotide in the range of multiple orders of magnitude (e.g., 1 to 10 to 100), preferably up to a ten-fold change, as compared to the release of octreotide from an implant matrix without the release rate modifier.

With the addition of a hydrophobic release rate modifier such as hydrophobic ethyl heptanoate, to the sustained release delivery system, and formation of the implant matrix through interaction of the flowable composition and body fluid, the release rate of octreotide can be slowed. Hydrophilic release rate modifiers such as polyethylene glycol may increase the release of the octreotide. By an appropriate choice of the polymer molecular weight in combination with an effective amount of the release rate modifier, the release rate and extent of release of a octreotide from the implant matrix may be varied, for example, from relatively fast to relatively slow.

Useful release rate modifiers include, for example, organic substances which are water-soluble, water-miscible, or water insoluble (i.e., hydrophilic to hydrophobic).

The release rate modifier is preferably an organic compound which is thought to increase the flexibility and ability of the polymer molecules and other molecules to slide past each other even though the molecules are in the solid or highly viscous state. Such an organic compound preferably includes a hydrophobic and a hydrophilic region. It is preferred that a release rate modifier is compatible with the combination of polymer and organic liquid used to formulate the sustained release delivery system. It is further preferred that the release rate modifier is a pharmaceutically-acceptable substance.

Useful release rate modifiers include, for example, fatty acids, triglycerides, other like hydrophobic compounds, organic liquids, plasticizing compounds and hydrophilic compounds. Suitable release rate modifiers include, for example, esters of mono-, di-, and tricarboxylic acids, such as 2-ethoxyethyl acetate, methyl acetate, ethyl acetate, diethyl phthalate, dimethyl phthalate, dibutyl phthalate, dimethyl adipate, dimethyl succinate, dimethyl oxalate, dimethyl citrate, triethyl citrate, acetyl tributyl citrate, acetyl triethyl citrate, glycerol triacetate, di(n-butyl) sebecate, and the like; polyhydroxy alcohols, such as propylene glycol, polyethylene glycol, glycerin, sorbitol, and the like; fatty acids; triesters of glycerol, such as triglycerides, epoxidized soybean oil, and other epoxidized vegetable oils; sterols, such as cholesterol; alcohols, such as C₆-C₁₂ alkanols, 2-ethoxyethanol, and the like. The release rate modifier may be used singly or in combination with other such agents. Suitable combinations of release rate modifiers include, for example, glycerin/propylene glycol, sorbitol/glycerine, ethylene oxide/propylene oxide, butylene glycol/adipic acid, and the like. Preferred release rate modifiers include dimethyl citrate, triethyl citrate, ethyl heptanoate, glycerin, and hexanediol.

The amount of the release rate modifier included in the flowable composition will vary according to the desired rate of release of the octreotide from the implant matrix. Preferably, the sustained release delivery system contains about 0.5-30%, preferably about 5-10%, of a release rate modifier.

Other solid adjuvants may also be optionally combined with the sustained release delivery system to act as carriers, especially isolation carriers. These include additives or excipients such as a starch, sucrose, lactose, cellulose sugar, mannitol, maltitol, dextran, sorbitol, starch, agar, alginates, chitins, chitosans, pectins, tragacanth gum, gum arabic, gelatins, collagens, casein, albumin, synthetic or semi-synthetic polymers or glycerides, and/or polyvinylpyrrolidone.

Additional adjuvants may include oils such as peanut oil, sesame oil, cottonseed oil, corn oil and olive oil as well as esters of fatty acids such as ethyl oleate, isopropyl myristate, fatty acid glycerides and acetylated fatty acid glycerides. Also included are alcohols, such as, but not limited to, ethanol, isopropyl alcohol, hexadecyl alcohol, glycerol and propylene glycol. Ethers, such as but not limited to, poly(ethyleneglycol), petroleum hydrocarbons such as mineral oil and petrolatum may also be used in the formulations. Pectins, carbomers, methyl cellulose, hydroxypropyl cellulose, hydroxypropyl methyl cellulose or carboxymethyl cellulose may also be included. These compounds can serve as isolation carriers by coating the octreotide thereby preventing its contact with the organic solvent and other ingredients of the flowable composition. As isolation carriers, these compounds also help lower the burst effect associated with the coagulation of the flowable composition in situ.

Optionally, other compounds such as, but not limited to, stabilizers, antimicrobial agents, antioxidants, pH modifiers, bioavailability modifiers and combinations of these are included. Emulsifiers and surfactants such as fatty acids, or a non-ionic surfactants including natural or synthetic polar oil, fatty acid esters, polyol ethers and mono-, di- or tri-glycerides may also be included.

### The Implant

When the implant of the invention is formed, the implant has the physical state of a solid or a gel. The solid embodiments may be rigid so that they cannot be flexed or bent by squeezing them between the fingers or they may be flexible or bendable so that they can be compressed or flexed out of original shape by squeezing between the fingers (i.e., a low amount of force). The gel embodiments may be jelly-like in consistency and will flow under pressure. The thermoplastic polymer functions as a matrix in these embodiments to provide integrity to the single body solid or gel and to enable controlled release of the bioactive agent upon implantation.

The thermoplastic polymer matrix is preferably a solid matrix and especially preferably is microporous. In an embodiment of the microporous solid matrix, there is a core surrounded by a skin. The core preferably contains pores of diameters from about 1 to about 1000 microns. The skin preferably contains pores of smaller diameters than those of the core pores. In addition, the skin pores are preferably of a size such that the skin is functionally non-porous in comparison with the core.

Because all of the components of the implant are biodegradable or can be swept away from the implant site by body fluid and eliminated from the body, the implant eventually disappears. Typically the implant components complete their biodegradation or disappearance after the octreotide has been essentially completely released. The structure of the thermoplastic polymer, its molecular weight, the density and porosity of the implant and the body location of the implant all affect the biodegradation and disappearance rates.

The implant is typically formed subcutaneously in a patient. It can be molded in place upon injection to provide comfort to the patient. The implant volume typically may be between 0.25 mL to 2 or 3 mL in size.

### Therapeutic Use

Surprisingly, it has been discovered that the sustained release delivery system according to the present invention is more effective in delivering octreotide than the Sandostatin LAR® product. Specifically, as shown in the Examples below, the blood levels of octreotide obtained with the sustained release delivery system of the present invention are higher at extended times in humans compared with those produced by the Sandostatin LAR® product, and also at the three month point in humans, compared to the value reported in the literature for the Sandostatin LAR® product.

Many of the advantages of this invention (e.g. superior release kinetics with minimal burst, increased duration of drug release with less frequent injections; markedly improved bioavailability; improved local tissue tolerance due to a small injection volume and the ability to use of a subcutaneous injection rather than intramuscular injection) are useful for the treatment of eye diseases. This includes eye diseases that involve excessive cellular proliferations, including but not limited to neovascular diseases of the eye, such as choroidal neovascularization, as occurs in age related macular degeneration, and retinal neovascularization, as occurs in diabetic retinopathy.

In general, any disease which may be ameloriated, treated, cured or prevented by administration of somatostatin or a somatostatin analog may be treated by administration of the flowable composition of the invention. These diseases relate to those having at least a partial basis in hypersecretion of growth hormone or somatotropin, imbalance in pathways involving insulin, glucagon and/or somatotropin, imbalance or malconditions involving somatostatin and/or somatotropin receptors, and malconditions associated with gastrointestinal ailments. The following specific malconditions are exemplary of such diseases. These may all be treated by appropriate, effective administration of a flowable composition of the invention formulated to deliver an effective amount of octreotide. These malconditions include:
a. Symptomatic control of diarrhea associated with carcinoid syndrome and vasoactive intestinal peptide (VIP) tumors;
b. Treatment of neuroendocrine tumors;
c. Acromegaly;
d. Symptomatic control of diarrhea associated chemotherapy-induced diarrhea;
e. Pancreatitis;
f. Bleeding esophageal varices;
g. Treatment of fluid accumulation associate with portocaval shunting;
h. Irritable bowel syndrome;
i. Anti-seizure medication;
j. Reduction in the formation of advanced glycation end (AGE) products (e.g. Hemoglobin A1C) in diabetic patients, which reduces the risk of diabetic complications;
k. Neovascular proliferative eye diseases (specific examples given in separate list below);
l. Other types of proliferative eye diseases (specific examples given in separate list below);

Examples of neovascular proliferative eye diseases that may be treated by a flowable composition of the invention include:
a. Retinal neovascularization in patients with diabetic retinopathy (with or without associated macular edema; with or without pre-retinal hemorrhage; with or without retinal detachment);
b. Retinal neovascularization as in patients with retinopathy of prematurity;
c. Choroidal neovascularization in patients with the wet form of age-related macular degeneration (with or without macular edema; with or without hemorrhage; with or without retinal detachment);
d. Choroidal neovascularization in patients with ocular and systemic diseases other than age-related macular degeneration;
e. Corneal neovascularization;

Examples of other types of proliferative eye diseases that may be treated by a flowable composition of the invention include:
a. Fibroblastic proliferations: Proliferative vitreoretinopathy or pterygium;
b. Autoimmune and inflammatory conditions: Graves' ophthamopathy with periocular and/or intraocular lymphocytic proliferation;
c. optic neuritis; any type of uveitis, iridocyclitis or scleritis caused by lymphocytic or monocytic cell proliferation;
d. Hematolymphoid neoplasms: intraocular lymphoma or leukemia;
e. Solid tumors: retinoblastoma, melanoma, rhabdomyosarcoma, embryonal sarcoma, metastatic malignant solid tumors or any other malignant or benign intraocular tumor; any oncogenic neovascularization of the eye.

Diabetic eye diseases that may be treated by a flowable composition of the invention include:
a. Non-proliferative retinopathy;
b. Early proliferative, non-high risk, retinopathy;
c. Proliferative retinopathy;
d. Severe retinopathy in patients who have failed photocoagulation;
e. Diabetic macular edema, including custoid macular edema;

The use of the flowable composition to treat diabetic eye conditions includes stand alone therapy, and combinations with other treatments. Examples include:
a. Laser photocoagulation therapy;
b. Locally injected steroids including intravitreal, retro-bulbar, sub-conjunctival and sub-Tenon injections of any steroidal compound.

The flowable composition of the invention may also be used as a stand alone therapy to treat CNV associated with many eye diseases and syndromes such as AMD. Such malconditions include for example:
a. Wet age-related macular degeneration “AMD” (including predominantly classic AMD, minimally classic AMD and occult AMD subtypes). AMD is the major disease associated with CNV lesions;
b. CNV lesions also develop in other conditions of the eye: pathologic myopia, angioid streaks, presumed ocular histoplasmosis syndrome (POHS), serous choroiditis, optic head drus en, idiopathic central serous chorioretinopathy, retinal coloboma, Best's disease, retinitis pigmentosa with exudates, serpiginous choroiditis, Behcet's syndrome, chronic uveitis, acute multifocal posterior placoid pigment epitheliopathy, birdshot chorioretinopathy, choroidal rupture, ischemic optic neuropathy, chronic retinal detachment, other conditions of the posterior segment of the eye.

The flowable composition of the invention may also be used as a treatment for CNV lesions in combination with other treatments, such as by combination with:
a. Photodynamic therapy (e.g. verteporfin (Visudyne, QLT, Inc.), SnET2 (etiopurpurin, Miravant, Inc.);
b. Locally injected anti-angiogenic agents. For example, intravitreal or subconjunctival anti-VEGF agents: Macugen/Eyetech, Pharmaceuticals, Inc; Lucentis/Genentech, Inc.; and VEGF Trap/Regeneron Pharmaceuticals, Inc.;
c. Locally injected angiostatic steroids (e.g. anecortave, Retanne/Alcon) which is administered as a sub-Tenon injection; or any corticosteroid that is administered locally to the ocular tissues (e.g. triamcinolone);
d. Systemic therapies for CNV, such as squalamine [Genaera, Inc] and other systemically administered anti-angiogenic agents (e.g. Avastin).

Additional malconditions susceptible to ameloriation, prevention or cure by treatment with octreotide include ocular manifestations of thyroid disease (i.e. Graves disease, Hashimoto's thyroiditis or other causes of hyperthyroidism) (See the references Krassas, G. E. et al, 1998; Pasquali, D. et al, 2002). The use of the flowable composition in the treatment of thyroid related ocular disease include its use as a stand alone therapy, and its use in combination with other treatments, such as steroids and other systemic immunosuppressive agents.

Further malconditions treatable with the flowable composition of the present invention include cystoid macular edema (Kuijpers, R. et al, 1998; Rothnova, A. et al, 2002), and visual field defects associated with pituitary adenomas that compress the optic nerve (e.g. in patients with acromegaly) (McKreage, K. et al, 2003).

### Dosages

The amount of flowable composition administered will typically depend upon the desired properties of the controlled release implant. For example, the amount of flowable composition can influence the length of time in which the octreotide is released from the controlled release implant. Specifically, in one embodiment of the present invention, the composition can be used to formulate a one month delivery system of octreotide. In such an embodiment, about 0.20 mL to about 0.40 mL of the flowable composition can be administered. Alternatively, in another embodiment of the present invention, the composition can be used to formulate a three month delivery system of octreotide. In such an embodiment, about 0.75 mL to about 1.0 mL of the flowable composition can be administered.

The amount of octreotide within the flowable composition and the resulting implant will depend upon the disease to be treated, the length of duration desired and the bioavailability profile of the implant. Generally, the effective amount will be within the discretion and wisdom of the patient's attending physician. Guidelines for administration include dose ranges of from about 100 to 5000 micrograms of octreotide per day as applied for proliferative and non-proliferative eye diseases. The typical flowable composition effective for such sustained delivery over a 1 month period will contain from about 5 to about 100 mg of octreotide per ml of total volume of flowable composition. The injection volume will range from 0.2 to 1.5 mL per implant. The typical flowable composition effective for such sustained delivery of a 3 month period will contain from about 12 to about 30 mg of octreotide per ml of total volume of flowable composition. The injection volume will range from 0.75 to 1.0 mL per implant. The polymer formulation will be the primary factor for obtaining the longer sustained release, as discussed above.

All publications, patents, and patent documents are incorporated by reference herein, as though individually incorporated by reference. The invention will now be illustrated with the following non-limiting examples.

The following Examples employ the ATRIGEL® formulation of poly(lactide-coglycolide) and N-methylpyrrolidone in combination with octreotide as the flowable composition.

### EXAMPLES

In the following Examples, ATRIGEL®/Octreotide refers to ATRIGEL®/Octreotide formulations; ATRIGEL® is a registered Trademark of QLT-USA, Fort Collins, Colo. The particular form of ATRIGEL® product used in these examples is provided with the examples. Unless otherwise indicated, the ATRIGEL® product is the thermoplastic polymer poly(lactide-coglycolide) (PLG) or the thermoplastic polymer poly(lactide-coglycolide extended with 1,6-hexane diol) (PLGH) in the organic solvent N-methyl-2-pyrrolidone. Sandostatin LAR® is used to refer to Sandostatin LAR® product; Sandostatin LAR® is a registered Trademark of Novartis AG, Basel, Switzerland.

Earlier attempts to solve the problem and the limitations or deficiencies of somatostatin problems have resulted in significant drawbacks. Sandostatin LARD product is a 30-day depot suspension of octreotide encapsulated in microparticles of poly (DL lactide-coglycolide) glucose. The microparticles of octreotide are suspended in an inert carrier rendering the suspension capable of being injected into the body to form microparticle implant. This Sandostatin LAR® depot has many drawbacks. These include: a) Poor pharmacokinetics with a lag phase of 7-10 days; b) Low bioavailability; c) Large injection volume that requires an IM route of administration; d) Severe injection site tissue reactions: muscle necrosis, acute inflammation with neutrophilic infiltration; e) scaring with chronic use; and f) Difficult preparation and administration with frequent needle clogging.

The above-mentioned shortcomings limit the usefulness, and in some cases adversely affect the product performance of Sandostatin LAR® product for all of its clinical applications. The above-mentioned shortcomings are particularly limiting in the case of ocular diseases. Of particular importance, effective treatment of diabetic retinopathy using octreotide requires multiple daily subcutaneous injections of Sandostatin solution with total daily doses between 200 and 5,000 micrograms (See the references in the reference section: Boem, B. O. et al, 2001; Grant, M. B. et al 2000; Grant, M. B. et al 2002). Indeed, it is not clear whether the sustained release depot, Sandostatin LAR® product, will provide sufficient drug exposure to be an effective treatment for diabetic retinopathy.

The flowable composition of the present invention solves these problems of bioavailability, pharmacokinetics, safety and convenience. As demonstrated below, the flowable composition of the invention provides higher bioavailability, enhanced release kinetics, lower volume of injection and the opportunity to use the subcutaneous or intravitreal routes of administration rather than the IM route of administration (volumes in excess of 1 mL of Sandostatin LAR® product must be injected intramuscularly). The flowable composition of the invention provides delivery volumes that are as little as 1/10^{th} the volume of Sandostatin LAR® product.

In addition as demonstrated by the clinical results provided below, the flowable compositions of the invention have no lag phase, continuous therapeutic plasma levels and potentially greater exposure to the target tissues, such as ocular neovessels. The 1- and 3-month flowable compositions provide an alternative drug delivery technology that addresses these as well as several other drawbacks of currently marketed somatostatin analogues and related products in development.

The advantages of the approach using the flowable composition of the invention to solve these problems include: a) Rapid therapeutic response—no lag time; b) Subcutaneous injection (Patient Friendly); c) Less pain; d) No muscle damage and scarring; e) Smaller-gauge needles; f) Less volume—1/10 of the Sandostatin LAR®product; g) Ease-of-administration; h) Quick and easy preparation; i) No clogging of the needle; and j) Removable up to eight weeks (unlike microspheres).

Furthermore, the advantages of the application of the 3-Month flowable composition for treatment of retinal and choroidal neovascularization include:
a) Meeting the more stringent product requirements for ophthalmic products as compared to other medical products;
b) Obtaining the required higher octreotide exposure to inhibit blood vessel growth;
c) Delivering much higher bioavailability compared to Sandostatin LAR®;
d) No lag phase—immediate therapeutic levels with no gaps;
e) Extreme safety—minimizing injection site reaction is important;
f) Subcutaneous rather than IM injection (˜ 1/10 volume of Sandostatin LAR® product);
g) No muscle damage or scarring, negligible risk of suppuration or deep tissue infection; and
h) Convenience of preparation and administration.

As a result, the flowable compositions of the invention provide superior pharmacokinetics and higher bioavailability relative to other known delivery systems providing octreotide. These features represent improvements regardless of the particular application, i.e. any somatostatin responsive disease. However, these kinetic improvements may be required for success of the products when used in ocular applications. That is because higher and more constant therapeutic levels are required to penetrate the blood-ocular-barrier and to block neovascularization in ocular tissues.

The flowable compositions provide continuous therapeutic levels that may improve efficacy for many applications, but it may be required to effectively inhibit pathological neovascularization in the anterior and posterior segments of the eye.

The flowable compositions provide a safer, more convenient product that can be injected less frequently. These features affect all applications.

The data summarized in the following examples indicate that the flowable composition of the invention has much higher bioavailability as compared to Sandostatin LAR® product. The two products have been tested side-by-side in multiple pre-clinical studies as well as a Phase 1 safety and pharmacology study conducted in normal volunteers. The data show that there is no lag phase. In contrast to Sandostatin LAR® product, the flowable composition provides immediate therapeutic levels with no gaps.

In addition, the data summarized in the attachment also indicate that the flowable composition of the invention has a much smaller injection volume as compared to Sandostatin LAR® product. For this reason, the flowable composition of the invention can be administered using a subcutaneous injection rather than an intramuscular injection. This difference is much more than a matter of patient convenience. Indeed, in experiments performed in rats, rabbits and dogs, we have repeatedly found that intramuscular injections of Sandostatin LAR® product produce severe acute tissue reactions, characterized by muscle necrosis and acute inflammation with neutrophilic infiltration (sterile abcess). These observations are corroborated by the clinical experience with Sandostatin LAR® product, which is well known to cause muscle loss and scarring in the buttocks of patients being treated for chronic conditions, such as acromegaly and carcinoid syndrome. The fact that the flowable composition can be administered by a subcutaneous injection means that there is negligible risk of suppuration or deep tissue infection. This is a critical advantage in the setting of diabetic retinopathy, because these patients are susceptible to infections.

### Example 1

### Evaluation of the 84-Day Release Kinetics of Four ATRIGEL® Formulations Containing 12% Octreotide Citrate Following a Single Subcutaneous Administration in Male Rats

### Summary

The purpose and primary objective of this study was to evaluate the 84-day release kinetics of four modified ATRIGEL® formulations, containing 12% octreotide citrate administered subcutaneously (SC) in rats utilizing implant retrieval and subsequent reversed phase high performance liquid chromatography (RP-HPLC). A secondary objective was to collect blood for plasma analysis of octreotide. A final objective was to evaluate test sites macroscopically for tissue reactions and test article (TA) characteristics.

In this 84-day study, four ATRIGEL® formulations were tested in one hundred and twenty male rats with thirty rats per treatment group. On Day 0, each animal received one 100 μL (approximate) SC injection of appropriate TA containing approximately 12 mg octreotide citrate in the dorsal thoracic (DT) region. On Days 1, 7, 21, 35, 56, and 84, five rats per group were anesthetized and bled (up to 5 mL) via cardiac puncture then euthanized by CO₂. Plasma octreotide levels were analyzed via liquid chromatography/mass spectrometry/mass spectrometry (LC/MS/MS) by ABC Laboratories, Inc. (Columbia, Mo.). TAs were retrieved for subsequent RP-HPLC analysis to determine their octreotide content. Macroscopic SC tissue reaction, relative to each TA, was evaluated by gross examination of the implants and the surrounding tissue.

The data demonstrated Group II, 12% Octreotide citrate suspended in [50% 85/15 PLG (InV 0.25) and 50% NMP], yielded a low burst (18.4%) and slow release rate of octreotide (88.6% released at Day 84). All of the test articles had low burst at Day 1 with a range of 10.2% to 30.2%. Group IV, 12% octreotide formulated with a blend of 85/15 PLGH and 65/35 PLG polymers exhibited the lowest burst (10.2%), while Group III, formulated with a blend of 85/15 PLGH and PLG polymers, demonstrated the highest burst (30.2%). All test articles showed sustained release of octreotide, with Group II exhibiting the slowest release rate (88.6% released at Day 84). Plasma octreotide analysis of Groups I and II indicated that maximum plasma octreotide concentrations (Cₘₐₓ) were reached at Day 1 (tₘₐₓ), then dropped gradually to a relatively steady level. The Cₘₐₓ was 114.4 ng/mL and 176.1 ng/mL for Groups I and II, respectively. Both groups remained higher than therapeutic plasma levels (0.3 ng/mL) (Marbach, P., Briner U., Lemaire M., Schweitzer A. and Terasaki T., From Somatostatin to Sandostatin: Pharmacodynamics and Pharmacokinetics, Metabolism, Vol 41, No. 9, Suppl. 2 (September), 1992: pp 7-10) throughout the study. Tissue irritation was minimal to mild in all groups on Day 1 with decreased irritation through Day 21. The results of this study indicate that an ATRIGEL®/Octreotide formulation with high polymer loading and a low inherent viscosity polymer vehicle provided an acceptable three month delivery of octreotide. A clarification throughout these examples is that octreotide citrate refers to octreotide acetate+citric acid.

### Introduction

Octreotide is a synthetic, eight amino acid peptide marketed by Novartis. The primary indication for octreotide is for the treatment of acromegaly caused by hypersecretion of growth hormone, and is indicated for the symptomatic control of metastatic carcinoid and vasoactive intestinal peptide-secreting tumors. The current clinical formulations are administered as subcutaneous daily injections (Sandostatin®), or as a single one-month sustained-release intramuscular depot (Sandostatin LAR® [Long Acting Release]). The one-month depot product is a microparticulate formulation in which the drug is encapsulated in microspheres that are prepared from glucose and poly(DL-lactide-co-glycolide) [PLG] polymers.

The ATRIGEL® drug delivery system is a biodegradable polymeric delivery system that can be injected as a liquid. Upon injection of the formulation, the polymer solidifies encapsulating the drug. As the process of biodegradation begins, the drug is slowly released. The release rate of drugs from this type of delivery system can be controlled by the type and molecular weight of the polymer and drug load of the constituted product. Therefore the system can be tailored to meet the needs of the patient.

### Materials and Methods

This was a single dose in vivo study designed to determine the 84-day release kinetics of octreotide delivered from four modified ATRIGEL® formulations injected SC into rats.

The rat is an acceptable model for SC injections since a large database is available in the literature. This procedure duplicates an anticipated route of administration for use in humans. Tissue culture techniques are not available that duplicate the release kinetics that occur in the living animal. The polymer system has been tested extensively and a large database of information is archived on its safety. A significant pain response was not anticipated in this study. No alternative methods were advised.

All percentages are weight to weight (w/w) and all inherent viscosities (InV) are in units of dL/g. A clarification throughout this report is that octreotide citrate refers to octreotide acetate+citric acid.

### Test Article Identification

1. 12% Octreotide citrate suspended in [45% 65/35 PLG (InV 0.36) and 55% NMP].
2. 12% Octreotide citrate suspended in [50% 85/15 PLGH (InV 0.25) and 50% NMP].
3. 12% Octreotide citrate suspended in [25% 85/15 PLGH (InV 0.25)+25% 85/15 PLG (InV 0.22) and 50% NMP].
4. 12% Octreotide citrate suspended in [30% 85/15 PLGH (InV 0.25)+20% 65/35 PLG (InV 0.36) and 50% NMP].
   Control: Not applicable.

### Manufacturer Information

| Substance | Manufacturer | Lot# |
|---|---|---|
| Octreotide acetate | Bachem | 110702-003 |
| Citric Acid | Fisher | 006630 |
| NMP | | TN-013102-000 |
| 65/35 PLG 0.36 | Birmingham Polymer | D95080 |
| | Industries (BPI) | |
| 85/15 PLGH 0.25 | Alkermes | 02-012-39 |
| 85/15 PLG 0.25 | Adsorbable Polymer | APT-1220101-1 |
| | Technologie (APT) | |

### Formulation Preparation

A. Preparation of Polymer Solutions

Polymer stock solutions were prepared by weighing a known amount of each polymer solid into individual 20 mL scintillation vials. A known amount of NMP was added to each polymer and the mixture placed on a jar mill. The vials were mixed overnight, producing a visually clear polymer solution. The weight of polymer and NMP in each solution is tabulated below.

| | Polymer Wt. | NMP | Wt. % Polymer in |
|---|---|---|---|
| Polymer | (g) | Wt. (g) | Solution |
| 1. 65/35 PLG (InV 0.36) | 2.7010 | 3.2963 | 45.03 |
| 2. 85/15 PLGH (InV 0.25) | 5.0000 | 5.0160 | 49.92 |
| 3. 85/15 PLGH (InV 0.25) + | 2.5025 | | 24.99 |
| 85/15 PLG (InV 0.25) | 2.5068 | 5.0035 | 25.04 |
| 4. 85/15 PLGH (InV 0.25) + | 1.8074 | | 29.98 |
| 65/35 PLG (InV 0.36) | 1.2020 | 3.0187 | 19.94 |

B. Preparation of Octreotide Acetate+Citric Acid Mixture

Octreotide acetate and citric acid mixture was prepared by dissolving 3.5006 g of octreotide acetate and 0.6595 g citric acid into 33 mL HPLC grade water. The solution was stirred until all solids were in solution. The weights used above were derived from a calculated 1:1 ratio of octreotide to citric acid. The solution was frozen at −86° C. for one hour then lyophilized for two days. The drug syringe filling solution was prepared by weighing 2.4307 g of the octreotide acetate+citric acid mixture into a 40 mL scintillation vial. Approximately 13.5 g of HPLC-grade water was weighed into a beaker. The 40 mL vial was placed on a balance, tared to zero, and water was added to the vial until the weight was 13.4994 g.

C. Preparation of A-B Syringes

Seven syringe pairs were prepared for each group in the study. Each pair of syringes contained approximately 635.5 mg of formulation. The B syringes (containing drug) were prepared by pipetting 500 mg of the octreotide acetate+citric acid syringe filling solution into 1.25 mL BD male syringes. B syringes were prepared by weighing 559.2 mg of polymer stock solution into 1 mL female syringes. The amount of each component weighed into the syringes and the weight percent of octreotide acetate+citric acid mixture in each formulation is listed below.

| | | | Wt (mg) | Wt (mg) of | Wt % of Octreotide |
|---|---|---|---|---|---|
| | | | of | Octreotide | acetate + citric acid |
| | | | polymer | acetate + citric | mixture in |
| Group# | Polymer solution | | solution | acid mixture | formulation |
| Group I | 45% 65/35 PLG | Pair 1 | 559.8 | 76.3 | 11.99 |
| | (InV 0.36) | Pair 2 | 559.6 | 76.3 | 12.00 |
| | | Pair 3 | 560.1 | 76.3 | 11.99 |
| | | Pair 4 | 559.3 | 76.3 | 12.00 |
| | | Pair 5 | 558.3 | 76.3 | 12.02 |
| | | Pair 6 | 558.9 | 76.3 | 12.01 |
| | | Pair 7 | 559.5 | 76.3 | 12.00 |
| Group II | 50% 85/15 | Pair 1 | 558.2 | 76.3 | 12.03 |
| | PLGH (InV | Pair 2 | 559.8 | 76.3 | 11.99 |
| | 0.25) | Pair 3 | 559.8 | 76.3 | 11.99 |
| | | Pair 4 | 558.3 | 76.3 | 12.02 |
| | | Pair 5 | 558.8 | 76.3 | 12.03 |
| | | Pair 6 | 560.2 | 76.3 | 11.99 |
| | | Pair 7 | 559.8 | 76.3 | 11.99 |
| Group III | 25% 85/15 | Pair 1 | 560.2 | 76.3 | 11.99 |
| | PLGH (InV | Pair 2 | 559.7 | 76.3 | 12.01 |
| | 0.25) + 25% | Pair 3 | 559.1 | 76.3 | 12.01 |
| | 85/15 PLG (InV | Pair 4 | 559.0 | 76.3 | 12.01 |
| | 0.25) | Pair 5 | 560.2 | 76.3 | 11.99 |
| | | Pair 6 | 558.9 | 76.3 | 12.01 |
| | | Pair 7 | 558.2 | 76.3 | 12.03 |
| Group IV | 30% 85/15 | Pair 1 | 560.5 | 76.3 | 11.98 |
| | PLGH (InV | Pair 2 | 558.5 | 76.3 | 12.02 |
| | 0.25) + 20% | Pair 3 | 560.1 | 76.3 | 11.99 |
| | 65/35 PLG (InV | Pair 4 | 560.4 | 76.3 | 11.98 |
| | 0.36) | Pair 5 | 560.8 | 76.3 | 11.98 |
| | | Pair 6 | 561.2 | 76.3 | 11.97 |
| | | Pair 7 | 560.7 | 76.3 | 11.98 |

### Experimental Design

In this 84-day study, four ATRIGEL® formulations were tested in 120 male rats with 30 rats per treatment group. On Day 0, each animal received one 100 μL (approximate) SC injection of appropriate TA containing approximately 12 mg octreotide citrate in the dorsal thoracic (DT) region. On Days 1, 7, 21, 35, 56, and 84, five rats per group were anesthetized and bled (up to 5 mL) via cardiac puncture then euthanized by CO₂. Plasma octreotide levels were analyzed via LC/MS/MS by ABC Laboratories, Inc. (Columbia, Mo.). TAs were retrieved for subsequent RP-HPLC analysis to determine their octreotide content. Macroscopic SC tissue reaction, relative to each TA, was evaluated by gross examination of the implants and the surrounding tissue.

| | | | Drug | | |
|---|---|---|---|---|---|
| | | Test | Dosage | | |
| | Number/ | Article/ | of Octreotide | Formulation | Termination |
| Group | Gender | Route | Citrate | Volume | Time Point |
| I | 30/M | 1/SC | 12 mg | 100 μL | Days 1, |
| | | | | | 7, 21, 35, |
| | | | | | 56, and 84 |
| II | 30/M | 2/SC | 12 mg | 100 μL | Days 1, 7, |
| | | | | | 21, 35, |
| | | | | | 56, and 84 |
| III | 30/M | 3/SC | 12 mg | 100 μL | Days 1, 7, |
| | | | | | 21, 35, |
| | | | | | 56, and 84 |
| IV | 30/M | 4/SC | 12 mg | 100 μL | Days 1, 7, |
| | | | | | 21, 35, |
| | | | | | 56, and 84 |

### Experimental Procedure

The in-life portion of the study lasted 84 days. A dose of 12.0 mg octreotide citrate was used. While under general isoflurane anesthesia, each rat was placed in sternal recumbency, its DT region shaved, and the injection site wiped with isopropanol. Each animal was administered a single 100 μL SC injection of appropriate TA in the DT region. During the course of the study the animals were observed for signs of overt toxicity and for any existing abnormalities, including redness, bleeding, swelling, discharge, bruising, and TA extrusion. Body weights were taken at administration and at termination.

On Days 1, 7, 21, 35, 56, and 84, five rats per group were anesthetized and bled via cardiac puncture. Following blood collection, each rat was euthanized with CO₂ and implants recovered. Representative photographs of the test sites were taken and precipitation characteristics of the implants were documented. Implants were placed in dry, labeled vials. Only mean and standard deviation were used in this study. There were no protocol modifications during the course of this study.

### Results and Discussion

Overt toxicity observations recorded during the course of the study noted evidence of diarrhea in several cages from each group on Day 0. On Day 1, soft stool was observed in Groups III and IV. Test site observations noted mostly redness and bruising at TA sites in all groups, primarily through Day 7. Some animals in all groups exhibited a black area at the injection site from Day 2 through Day 6, then a flaky scab was noted through Day 28.

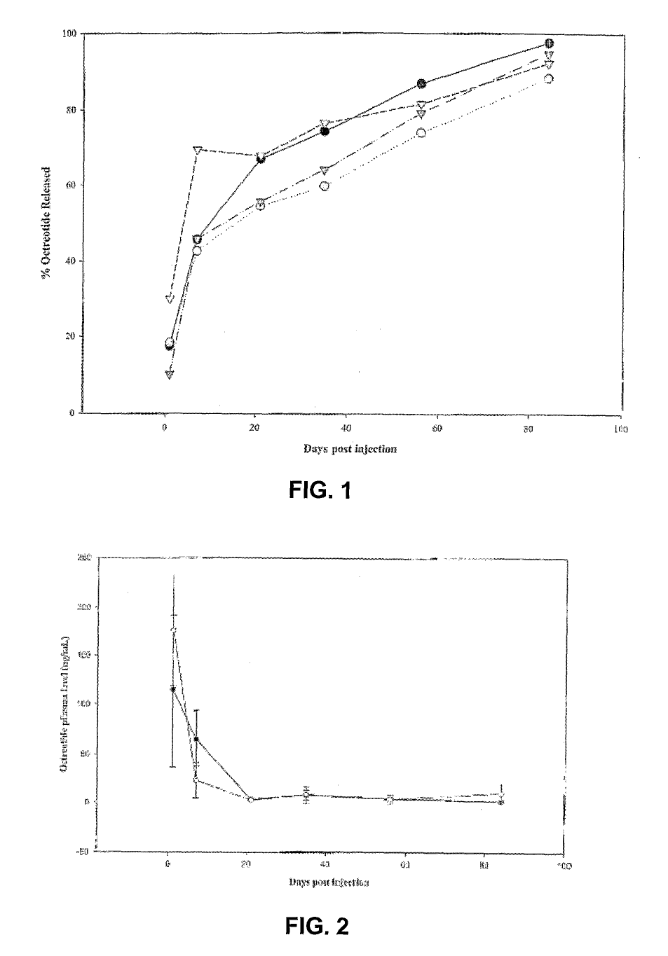

Table 1-1 and FIG. 1 illustrate the implant retrieval data. The data demonstrated low burst for all groups, with a range of 10.2% (Group IV) to 30.2% (Group III). All formulations released octreotide gradually with Group II exhibiting the slowest release (88.6% total drug load at 84 days post dosing). The combination of high polymer loading coupled with low InV of Group II (50% and InV 0.25) verses Group I (45% and InV 0.36) may have contributed to the slow release of the Group II formulation.

TABLE 1-1
Percent Octreotide Released After Subcutaneous Injection in Rats

| Test | Time | Mean | Standard |
|---|---|---|---|
| Article | Point | Percent | Deviation |
| Group I: 12% Octreotide acetate + citric | Day 1 | 17.6 | 4.6 |
| acid in 45% 65/35 PLG (InV 0.36)/55% | Day 7 | 45.7 | 6.7 |
| NMP, irradiated | Day 21 | 65.1 | 10.5 |
| | Day 35 | 74.5 | 3.4 |
| | Day 56 | 80.8 | 13.7 |
| | Day 84 | 97.7 | 0.6 |
| Group II: 12 Octreotide acetate + citric | Day 1 | 23.9 | 8.3 |
| acid in 50% 85/15 PLGH (InV 0.25)/50% | Day 7 | 42.7 | 7.2 |
| NMP, irradiated | Day 21 | 54.5 | 5.4 |
| | Day 35 | 59.9 | 3.7 |
| | Day 56 | 74.2 | 1.6 |
| | Day 84 | 88.6 | 2.9 |
| Group III: 12% Octreotide acetate + citric | Day 1 | 30.2 | 5.6 |
| acid in 25% 85/15 PLGH (InV 0.25) + | Day 7 | 69.6 | 4.0 |
| 25% 85/15 PLG (InV 0.25)/45% NMP, | Day 21 | 67.9 | 4.8 |
| irradiated | Day 35 | 76.6 | 9.1 |
| | Day 56 | 81.8 | 8.1 |
| | Day 84 | 92.4 | 1.2 |
| Group IV: 12% Octreotide acetate + citric | Day 1 | 10.2 | 3.3 |
| acid in 30% 85/15 PLGH (InV 0.25) + | Day 7 | 45.4 | 7.8 |
| 25% 65/35 PLG (InV 0.36)/45% NMP, | Day 21 | 55.8 | 6.8 |
| irradiated | Day 35 | 64.2 | 3.6 |
| | Day 56 | 79.4 | 3.2 |
| | Day 84 | 94.9 | 1.5 |

The polymers used in Group IV were a combination of the polymers used in Group I (65/35 PLGH InV 0.36) and Group II (85/15 PLGH InV 0.25) with a ratio of 2 to 3. A comparison among these three groups showed that Group II demonstrated the lowest release rate and Group I, the highest. Interestingly, the release rate of the blend group (Group IV) was between Groups I and II, and had the lowest burst at Day 1. A comparison between Group II and III suggested that blending 85/15 PLG (InV 0.25) into 85/15 PLGH (InV 0.25) gel increased the release rate of octreotide from the formulation.

Table 1-2 and FIG. 2 present the octreotide plasma level of each rat in Groups I and II. The mean plasma octreotide Cₘₐₓ for Groups I (114.4 ng/mL) and II (176.1 ng/mL) were reached on Day 1. Only Groups I and II were selected for plasma analysis due to the preferred release profiles.

TABLE 1-2
Plasma Octreotide Levels in Rats (Groups I and II) Following
Subcutaneous Injection of ATRIGEL ®/Octreotide Formulations

| | | Mean | |
|---|---|---|---|
| | | Octreotide | |
| Test | Time | in plasma | Standard |
| Article | Point | (ng/mL) | Deviation |
| Group I: 12% Octreotide acetate + | Day 1 | 114.4 | 77.8 |
| citric acid in 45% 65/35 PLG (InV | Day 7 | 65.4 | 28.0 |
| 0.36)/55% NMP, irradiated | Day 21 | 3.0 | 1.2 |
| | Day 35 | 3.9 | 5.2 |
| | Day 56 | 4.1 | 4.4 |
| | Day 84 | 1.7 | 0.9 |
| Group II: 12 Octreotide acetate + citric | Day 1 | 176.1 | 57.4 |
| acid in 50% 85/15 PLGH (InV 0.25)/ | Day 7 | 22.8 | 18.6 |
| 50% NMP, irradiated | Day 21 | 2.4 | 0.8 |
| | Day 35 | 8.4 | 8.4 |
| | Day 56 | 3.7 | 1.8 |
| | Day 84 | 10.2 | 9.5 |

### Conclusions

The data demonstrated Group II, 12% Octreoride citrate suspended in [50% 85/15 PLG (InV 0.25) and 50% NMP], yielded a low burst (18.4%) and slow release rate of octreotide (88.6% released at Day 84). All of the test articles had low burst at Day 1 with a range of 10.2% to 30.2%. Group IV, 12% octreotide formulated with a blend of 85/15 PLGH and 65/35 PLG polymers exhibited the lowest burst (10.2%), while Group III, formulated with a blend of 85/15 PLGH and PLG polymers, demonstrated the highest burst (30.2%). All test articles showed sustained release of octreotide, with Group II exhibiting the slowest release rate (88.6% released at Day 84). Plasma octreotide analysis of Groups I and II indicated that maximum plasma octreotide concentrations (Cₘₐₓ) were reached at Day 1 (tₘₐₓ), then dropped gradually to a relatively steady level. The Cₘₐₓ was 114.4 ng/mL and 176.1 ng/mL for Groups I and II, respectively. Both groups remained higher than therapeutic plasma levels (0.3 ng/mL) throughout the study (See P. Marbach, et al. “From Somatostatin to Sandostatin: Pharmacodymanics and Pharmacokinetics”, Metabolism, 1992, 41(9, supp. 2), pp. 7-10). Tissue irritation was minimal to mild in all groups on Day 1 with decreased irritation through Day 21. The results of this study indicate that an ATRIGEL®/Octreotide formulation with high polymer loading and a low inherent viscosity polymer vehicle provided an acceptable three month delivery of octreotide. A clarification throughout this example is that octreotide citrate refers to octreotide acetate+citric acid.

### Example 2

### Evaluation of the 85-Day Release Kinetics of Six ATRIGEL®/Octreotide Formulations Following a Single Subcutaneous Administration in Male Rats

### Summary

The purpose and primary objective of this study was to evaluate the 85-Day release kinetics of six modified ATRIGEL®/Octreotide formulations administered subcutaneously (SC) in rats, utilizing implant retrieval and subsequent reversed phase high performance liquid chromatography (RP-HPLC). A secondary objective was to collect blood for possible future plasma analysis of octreotide. A final objective was to evaluate test sites macroscopically for tissue reactions and test article (TA) characteristics.

In this 85-Day study, six ATRIGEL®/Octreotide formulations were tested in one hundred and eighty male rats with thirty rats per treatment group. On Day 0, Groups I, III, IV, V, and VI received one 100 μL (approximate) SC injection of appropriate TA containing approximately 9.6 mg octreotide in the dorsal thoracic (DT) region. Group II received one 100 μL (approximate) SC injection of formulation containing approximately 12 mg octreotide in the DT region. On Days 1, 7, 21, 35, 56, and 85, five rats per group were anesthetized and bled (up to 5 mL) via cardiac puncture. Following blood collection, each animal was euthanized by CO₂ and TAs were retrieved for subsequent RP-HPLC analysis to determine their octreotide content. Plasma octreotide levels were analyzed by Liquid Chromatography/Mass Spectrometry/Mass Spectrometry (LC/MS/MS) at ABC Laboratories (Columbia, Mo.). Macroscopic SC tissue reaction, relative to each TA, was evaluated by gross examination of the implants and the surrounding tissue.

The data show that an ATRIGEL® delivery system prepared with a blend of 65/35 PLG (InV 0.36) into 85/15 PLGH (InV 0.25) polymer solution (Groups III, IV and V) yields an acceptably low initial burst and release rate of octreotide over 85 days. The ratio of blending, however, had little effect on the release between these groups. Conversely, blending 50/50 PLGH (InV 0.30) into 85/15 PLGH (InV 0.25) increased the rate of release of octreotide greatly (Group VI). Increasing the drug load from 12% (Group I) to 15% (Group II) resulted in similar release profiles, while Group II displayed slightly lower burst at Day 1 (20.7±2.1%) versus Group I (27.2±3.6%) and lower release until Day 35, yet a faster release rate of octreotide after Day 35.

The pharmacokinetic (PK) analysis of Group I and Group II indicated that plasma concentrations of octreotide reached maximum levels (Cₘₐₓ) 24 hours post dosing. Plasma octreotide levels decreased during the first 21 days and then remained at relatively steady levels. Both groups had higher than therapeutic plasma octreotide levels (0.3 ng/mL) (Marbach, P., Briner U., Lemaire M., Schweitzer A. and Terasaki T., From Somatostatin to Sandostatin: Pharmacodynamics and Pharmacokinetics, Metabolism, Vol 41, No. 9, Suppl. 2 (September), 1992: pp 7-10) throughout the study. Group I plasma levels reached a Cₘₐₓ of 149.8±29.8 ng/mL on Day 1 and the lowest octreotide level of 3.4±0.7 ng/mL was seen on Day 85. Group II plasma levels reached a Cₘₐₓ of 141.4±58.6 ng/mL on Day 1 and the lowest octreotide level of 3.5±0.3 ng/mL was seen on Day 85. Minimal erythema was observed in one or two animals from Groups I-III, and VI on Day 1 and slight redness of the skin over the implant was noted in some animals in Groups I-IV. On Day 7, external scabs at the implant site were observed in one animal in Groups II and III and four of the five rats in Group IV.

### Introduction

Octreotide is a synthetic, eight amino acid peptide marketed by Novartis. The primary indication for octreotide is for the treatment of acromegaly caused by hypersecretion of growth hormone, and is indicated for the symptomatic control of metastatic carcinoid and vasoactive intestinal peptide-secreting tumors. The current clinical formulations are administered as subcutaneous daily injections (Sandostatin®), or as a single one-month sustained-release intramuscular depot (Sandostatin LAR® [Long Acting Release]). The one-month depot product is a microparticulate formulation in which the drug is encapsulated in microspheres that are prepared from glucose and poly(DL-lactide-co-glycolide) [PLG] polymers.

The ATRIGEL® drug delivery system is a biodegradable polymeric delivery system that can be injected as a liquid. Upon injection of the formulation, the polymer solidifies encapsulating the drug. As the process of biodegradation begins, the drug is slowly released. The release rate of drugs from this type of delivery system can be controlled by the type and molecular weight of the polymer, and drug load of the constituted product. Therefore the system can be tailored to meet the needs of the patient.

### Materials and Methods

This was a single dose in vivo study designed to determine the 85-day release kinetics of six modified ATRIGEL®/Octreotide formulations administered SC in rats. All percentages are weight to weight (w/w) and all inherent viscosities (InV) are in units of dL/g.

Test Article Identification:
1. 12% Octreotide+citric acid in [50% 85/15 PLGH (InV 0.25) and 50% NMP].
2. 15% Octreotide+citric acid in [50% 85/15 PLGH (InV 0.25) and 50% NMP].
3. 12% Octreotide+citric acid in [35% 85/15 PLGH (InV 0.25)+20% 65/35 PLG (InV 0.36) and 50% NMP].
4. 12% Octreotide+citric acid in [35% 85/15 PLGH (InV 0.25)+15% 65/35 PLG (InV 0.36) and 50% NMP].
5. 12% Octreotide+citric acid in [20% 85/15 PLGH (InV 0.25)+30% 65/35 PLG (InV 0.36) and 50% NMP].
6. 12% Octreotide+citric acid in [30% 85/15 PLGH (InV 0.25)+20% 50/50 PLGH (InV 0.30) and 50% NMP].
   Control: Not applicable.

### Manufacturer Information:

| Substance | Manufacturer | Lot# |
|---|---|---|
| Octreotide acetate | Bachem | 110702-003 |
| Citric Acid | Fisher | 006630 |
| NMP | | TN-013102-000 |
| 65/35 PLG (InV 0.37) | Birmingham Polymer | D95080 |
| | Industries (BPI) | |
| 85/15 PLGH (InV 0.25) | Alkermes | 02-012-39 |
| 50/50 PLGH (InV 0.30) | Boerhinger Ingelheim | 281366 |
| | (BI) | |

### Formulation Preparation

A. Preparation of Polymer Solutions

Polymer stock solutions were prepared by weighing a known amount of each polymer solid into individual 20 mL scintillation vials. A known amount of NMP was added to each polymer and the mixture placed on a jar mill. The vials were mixed overnight, producing a visually clear polymer solution. The polymer solutions were all γ-irradiated.

B. Preparation of Octreotide Acetate+Citric Acid Mixture

Octreotide acetate and citric acid mixture was prepared by dissolving 3.5006 g of octreotide acetate, and 0.6595 g citric acid into 33 mL HPLC grade water. The solution was stirred until all solids were in solution. The weights used above were derived from a calculated 1:1 ratio of octreotide to citric acid. The solution was divided into 7 separate scintillation vials, and frozen at −86° C. for 1 hour, then lyophilized for two days.

C. Preparation of A-B Syringes

Seven syringe pairs were prepared for Group II and six syringe pairs for the remaining groups in the study. The “B” syringes (male syringes) were prepared by weighing octreotide stock solution into 1.25 mL BD syringes, followed by lyophilization for 24 hours. “A” syringes (female syringes) were prepared by weighing polymer solution into 1 mL female syringes. Octreotide stock solution was prepared by weighing 3.8999 g octreotide acetate+citric acid mixture into a volumetric flask, then brought to 25 mL with HPLC water. The final concentration of the octreotide stock solution was 156 mg/mL. Each male syringe in Group II contained 112.5 mg drug mixture. The remaining groups contained 102.0 mg in each male syringe. Each female syringe in Group II contained 637.5 mg polymer gel and the other groups contained 748.0 mg polymer gel.

### Experimental Design

In this 85-day study, six ATRIGEL®/Octreotide formulations were tested in one hundred and eighty male rats with thirty rats per treatment group. On Day 0, Groups I, III, IV, V, and VI received one 100 μL (approximate) SC injection of appropriate TA containing approximately 9.6 mg octreotide in the dorsal thoracic (DT) region. Group II received one 100 μL (approximate) SC injection of formulation containing approximately 12 mg octreotide in the DT region. On Days 1, 7, 21, 35, 56, and 85, five rats per group were anesthetized and bled (up to 5 mL) via cardiac puncture. Following blood collection, each animal was euthanized by CO₂ and TAs were retrieved for subsequent RP-HPLC analysis to determine their octreotide content. Plasma octreotide levels were analyzed by LC/MS/MS at ABC Laboratories (Columbia, Mo.). Macroscopic SC tissue reaction, relative to each TA, was evaluated by gross examination of the implants and the surrounding tissue.

| | | Test | Drug | | |
|---|---|---|---|---|---|
| | Number/ | Article/ | Dosage | Formulation | Termination |
| Group | Gender | Route | of Octreotide | Volume | Time Point |
| I | 30/M | 1/SC | 9.6 mg | 100 μL | Days 1, |
| | | | | | 7, 21, 35, |
| | | | | | 56, and 85 |
| II | 30/M | 2/SC | 12 mg | 100 μL | Days 1, |
| | | | | | 7, 21, 35, |
| | | | | | 56, and 85 |
| III | 30/M | 3/SC | 9.6 mg | 100 μL | Days 1, |
| | | | | | 7, 21, 35, |
| | | | | | 56, and 85 |
| IV | 30/M | 4/SC | 9.6 mg | 100 μL | Days 1, |
| | | | | | 7, 21, 35, |
| | | | | | 56, and 85 |
| V | 30/M | 5/SC | 9.6 mg | 100 μL | Days 1, |
| | | | | | 7, 21, 35, |
| | | | | | 56, and 85 |
| VI | 30/M | 6/SC | 9.6 mg | 100 μL | Days 1, |
| | | | | | 7, 21, 35, |
| | | | | | 56, and 85 |

### Experimental Procedure

The in-life portion of the study lasted 85 days. A dose of 9.6 or 12 mg octreotide was used. While under general isoflurane anesthesia, each rat was placed in sternal recumbency, its DT region shaved, and the injection site wiped with isopropanol. Each animal was administered a single 100 μL SC injection of appropriate TA in the DT region. During the course of the study the animals were observed for signs of overt toxicity and for any existing abnormalities, including redness, bleeding, swelling, discharge, bruising, and TA extrusion. Body weights were taken at administration and at termination.

On Days 1, 7, 21, 35, 56, and 85, five rats per group were anesthetized and bled via cardiac puncture. Following blood collection, each rat was terminated with CO₂ and implants were recovered. Representative photographs of the test sites were taken and precipitation characteristics of the implants were documented. Implants were placed in dry, labeled vials.

Mean and standard deviation were used in this study. There were no protocol modifications during the course of this study.

### Results and Discussion

Overt toxicity observations recorded during the course of the study noted soft stool found in all Groups on Day 1. Test site observations noted redness, bruising and few instances of swelling at TA sites in all groups, primarily through Day 7. Animals exhibited a flaky scab at the injection site mainly from Day 2 through Day 14.

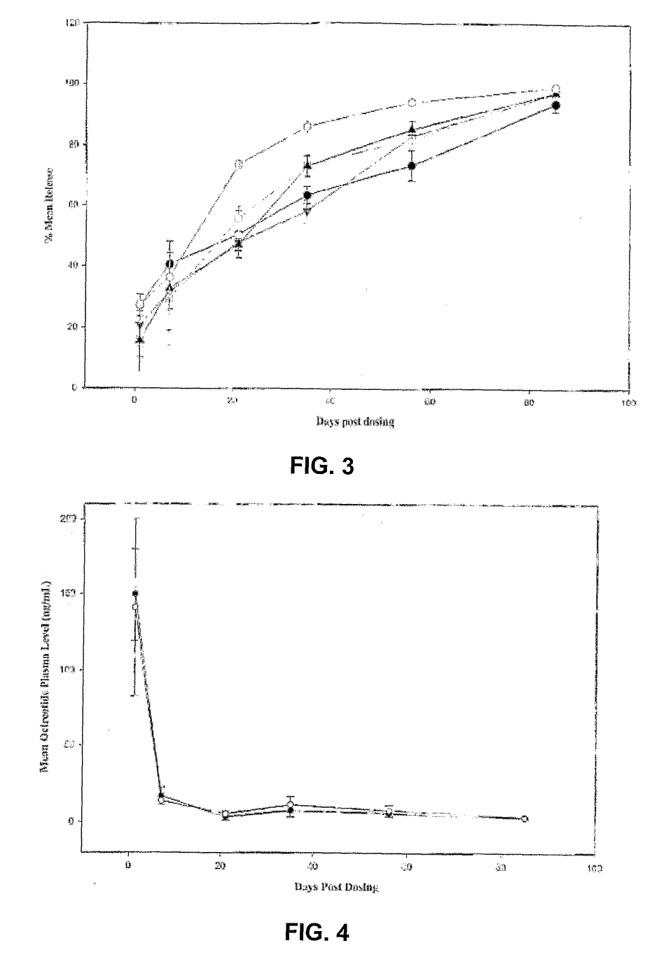

Table 2-1 illustrates the percentage of octreotide released from each formulation at each time point. The data demonstrated that all test articles had low burst at Day 1 with a range of 15.6% to 27.5% while Groups I through V showed sustained release of octreotide over the 85 days of the study. The mean release of octreotide for all formulations is depicted in FIG. 3.

TABLE 2-1
Percent of Octreotide Released Following SC Injection in Rats

| Test | Time | Mean % | Standard |
|---|---|---|---|
| Article | Point | Released | Deviation |
| Group I: 12% Octreotide acetate + | Day 1 | 27.2 | 3.6 |
| citric acid in 50% 85/15 PLGH (InV | Day 7 | 40.5 | 7.7 |
| 0.25)/50% NMP, irradiated | Day 21 | 50.4 | 7.9 |
| | Day 35 | 63.7 | 3.0 |
| | Day 56 | 73.6 | 4.9 |
| | Day 84 | 93.6 | 2.6 |
| Group II: 15% Octreotide acetate + | Day 1 | 20.7 | 2.1 |
| citric acid in 50% 85/15 PLGH (InV | Day 7 | 32.0 | 12.7 |
| 0.25)/50% NMP, irradiated | Day 21 | 47.8 | 2.5 |
| | Day 35 | 58.4 | 3.9 |
| | Day 56 | 83.0 | 1.4 |
| | Day 84 | 97.1 | 0.7 |
| Group III: 12% Octreotide acetate + | Day 1 | 17.2 | 5.2 |
| citric acid in 20% 85/15 PLGH (InV | Day 7 | 29.4 | 15.7 |
| 0.25) + 30% 65/35 PLG (InV 0.37)/ | Day 21 | 55.6 | 4.0 |
| 45% NMP, irradiated | Day 35 | 73.6 | 2.5 |
| | Day 56 | 81.9 | 2.6 |
| | Day 84 | 98.0 | 1.1 |
| Group IV: 12% Octreotide acetate + | Day 1 | 17.8 | 4.2 |
| citric acid in 30% 85/15 PLGH (InV | Day 7 | 37.5 | 4.8 |
| 0.25) + 20% 65/35 PLG (InV 0.37)/ | Day 21 | 50.3 | 3.3 |
| 50% NMP, irradiated | Day 35 | 76.2 | 4.6 |
| | Day 56 | 88.1 | 2.5 |
| | Day 84 | 97.2 | 1.6 |
| Group V: 12% Octreotide acetate + | Day 1 | 15.6 | 5.5 |
| citric acid in 55% 85/15 PLGH (InV | Day 7 | 32.7 | 6.8 |
| 0.25) + 15% 65/35 PLG (InV 0.37)/ | Day 21 | 47.0 | 1.8 |
| 50% NMP, irradiated | Day 35 | 73.4 | 3.5 |
| | Day 56 | 85.2 | 2.8 |
| | Day 84 | 97.3 | 0.7 |
| Group VI: 12% Octreotide acetate + | Day 1 | 27.5 | 3.4 |
| citric acid in 30% 85/15 PLGH (InV | Day 7 | 36.4 | 6.7 |
| 0.25) + 20% 50/50 PLGH (InV 0.30)/ | Day 21 | 74.0 | 1.2 |
| 50% NMP, irradiated | Day 35 | 86.1 | 1.8 |
| | Day 56 | 94.0 | 0.6 |
| | Day 84 | 99.2 | 0.1 |

Plasma levels of octreotide of Group I and II were selected for analysis.

The plasma levels of octreotide of Group I and II were analyzed, and summarized in Table 2-2. The mean plasma levels of these two groups are depicted in FIG. 4.

TABLE 2-2
Plasma Levels of Octreotide in Rats (Groups I and II)

| | | Mean | |
|---|---|---|---|
| | | Octreotide | |
| Test | Time | in plasma | Standard |
| Article | Point | (ng/mL) | Deviation |
| Group I: 12% Octreotide acetate + | Day 1 | 149.8 | 29.8 |
| citric acid in 50% 85/15 PLGH (InV | Day 7 | 17.2 | 5.7 |
| 0.25)/50% NMP, irradiated | Day 21 | 3.5 | 2.1 |
| | Day 35 | 7.9 | 4.3 |
| | Day 56 | 6.0 | 2.1 |
| | Day 84 | 3.4 | 0.7 |
| Group II: 15% Octreotide acetate + | Day 1 | 141.4 | 58.6 |
| citric acid in 50% 85/15 PLGH (InV | Day 7 | 14.2 | 2.7 |
| 0.25)/50% NMP, irradiated | Day 21 | 5.5 | 1.5 |
| | Day 35 | 11.7 | 5.1 |
| | Day 56 | 8.0 | 1.8 |
| | Day 84 | 3.5 | 0.3 |

### Conclusions

This study compared the release of octreotide from ATRIGEL® formulations containing 85/15 PLGH alone, blends of 65/35 PLG at three different levels (12, 13.5, and 15%), and a blend with 50/50 PLGH in the formulation. Two different drug loadings of 12 and 15% were also compared in the 85/15 PLGH formulation. The implant retrieval data showed that all of the formulations had an acceptable low initial burst of octreotide at Day 1 with a range of 15.6% to 27.5%. The polymer blend with 15% 65/35 PLG (Group IV) gave the lowest initial burst of 15.6%, although there did not appear to be a significant difference among the three 65/35 blends. The formulations of 85/15 PLGH with different drug loadings and the 50/50 PLGH blend also gave similar initial burst values, but slightly higher than the 65/35 PLG blends. All test articles showed sustained release of octreotide out to 85 days with only slight differences in the overall cumulative release rates between the 85/15 PLGH formulations with 12 and 15% drug loadings and those with the 65/35 PLG blends. However, the formulation (Group VI) containing the 50/50 PLGH showed a higher overall release rate than the other formulations. This effect was expected to some extent based upon the higher hydrophilicity and faster degradation of the 50/50 PLGH polymer compared to the more hydrophobic and slower degrading 65/36 PLG material.

Plasma analyses for octreotide concentration were conducted only for Group I and Group II animals to conserve costs. The data showed that the maximum octreotide plasma concentrations (Cₘₐₓ) were reached with 24 hours post dosing. Plasma octreotide levels had decreased significantly by Day 7 and remained at a relative steady level from Day 21 to Day 85. The two formulations gave almost the same plasma concentrations throughout the study reflecting the similarity of the implant retrieval release data.

Tissue irritation as determined by macroscopic evaluation was none to minimal for all groups. One or two animals from Groups I, II, III, and VI gave minimal erythema on Day 1, and a slight redness of the skin over the implant was noted in some animals in Groups I-IV. On Day 7, external scabs at the implant site were observed in one animal in Groups II and III, and in four of the five rats in Group IV.

In conclusion, the results of this study show that blending different levels of a 65/35 PLG polymer in an 85/15 PLGH ATRIGEL® formulation containing octreotide acetate/citrate has little effect on the release characteristics. However, blending the more hydrophilic and faster degrading 50/50 PLGH polymer into the same formulation gives a faster release of octreotide. Also, changing the concentration of the drug mixture from 12% to 15% did not seem to affect the release characteristics of the formulation containing the 85/15 PLGH polymer to any major extent.

### Example 3

### Evaluation of the 99-Day Release Kinetics of Three ATRIGEL®/Octreotide Formulations Following a Single Subcutaneous Administration in Male Rats

### Summary

The purpose and primary objective of this study was to evaluate the 99-day release kinetics of three modified ATRIGEL®/Octreotide formulations administered subcutaneously (SC) in rats, utilizing implant retrieval and subsequent reversed phase high performance liquid chromatography (RP-HPLC). A secondary objective was to collect blood for plasma analysis of octreotide. A tertiary objective was to evaluate test sites macroscopically for tissue reactions and test article (TA) characteristics.

In this 99-day study, three ATRIGEL®/Octreotide formulations were tested in one hundred and thirty-five male rats with forty-five rats per treatment group. On Day 0, each rat received one 100 μL (approximate) SC injection of formulation containing approximately 12 mg, 13.5 mg or 15 mg octreotide in the dorsal thoracic (DT) region. On Days 1, 4, 7, 14, 28, 60, 75, 90, and 99, five rats per group were anesthetized and bled (up to 5 mL) via cardiac puncture. Following blood collection, each animal was euthanized by CO₂ and TAs retrieved for subsequent RP-HPLC analysis to determine their octreotide content. Macroscopic SC tissue reaction, relative to each TA, was evaluated by gross examination of the implants and the surrounding tissue. Plasma was analyzed for octreotide content by Liquid Chromatography/Mass Spectrometry/Mass Spectrometry (LC/MS/MS) at ABC Laboratories (Columbia, Mo.).

The data demonstrate that all test articles had less than 10% burst at Day 1 and a similar slow release profile for 90 days. After Day 28, Group I (12% Octreotide acetate+citric acid suspended in [50% 85/15 PLGH (InV 0.27) and 50% NMP]) displayed the slowest release rate. On the other hand, Group III (15% Octreotide acetate+citric acid suspended in [50% 85/15 PLGH (InV 0.27) and 50% NMP].) demonstrated the fastest release rate of octreotide. All groups reached maximum plasma octreotide concentrations (Cₘₐₓ) at Day 1. Groups II and III exhibited slightly higher mean plasma octreotide levels at Day 1 versus Group I (24.6±5.6 ng/mL, 23.7±9.6 ng/mL versus 19.0±8.2 ng/mL, respectively). All groups revealed higher than therapeutic plasma octreotide levels (0.3 ng/mL) (Marbach, P., Briner U., Lemaire M., Schweitzer A. and Terasaki T., From Somatostatin to Sandostatin: Pharmacodynamics and Pharmacokinetics, Metabolism, Vol 41, No. 9, Suppl. 2 (September), 1992: pp 7-10) throughout the course of the study. Minimal tissue irritation was observed with few instances of minimal tissue irritation through Day 14.

### Introduction

Octreotide is a synthetic, eight amino acid peptide marketed by Novartis. The primary indication for octreotide is for the treatment of acromegaly caused by hypersecretion of growth hormone, and is indicated for the symptomatic control of metastatic carcinoid and vasoactive intestinal peptide-secreting tumors. The current clinical formulations are administered as subcutaneous daily injections (Sandostatin®), or as a single one-month sustained-release intramuscular depot (Sandostatin LAR® [Long Acting Release]). The one-month depot product is a microparticulate formulation in which the drug is encapsulated in microspheres that are prepared from glucose and poly(DL-lactide-co-glycolide) [PLG] polymers.

The ATRIGEL® drug delivery system is a biodegradable polymeric delivery system that can be injected as a liquid. Upon injection of the formulation, the polymer solidifies encapsulating the drug. As the process of biodegradation begins, the drug is slowly released. The release rate of drugs from this type of delivery system can be controlled by the type and molecular weight of the polymer, and drug load of the constituted product. Therefore the system can be tailored to meet the needs of the patient.

### Materials and Methods

This was a single dose in vivo study designed to determine the 99-day release kinetics of three modified ATRIGEL®/Octreotide formulations administered SC in rats. All percentages are weight to weight (w/w) and all inherent viscosities (InV) are in units of dL/g.

Test Article Identification:
1. 12% Octreotide acetate+citric acid suspended in [50% 85/15 PLGH (InV 0.27) and 50% NMP].
2. 13.5% Octreotide acetate+citric acid suspended in [50% 85/15 PLGH (InV 0.27) and 50% NMP].
3. 15% Octreotide acetate+citric acid suspended in [50% 85/15 PLGH (InV 0.27) and 50% NMP].
   Control Article There were no controls used in this study.

### Manufacturer Information:

| Substance | Manufacturer | Lot# |
|---|---|---|
| Octreotide acetate | Bachem | 110702-003 |
| Citric Acid | Fisher | 006630 |
| NMP | | TN-013102-000 |
| 85/15 PLGH (InV 0.27) | APT | A140-13 |

### Formulation Preparation

A. Preparation of Polymer Solutions

Polymer stock solutions were prepared by weighing a known amount of each polymer solid into individual 20 mL scintillation vials. A known amount of NMP was added to each polymer and the mixture placed on a jar mill. The vials were mixed overnight until a visually clear polymer solution was produced. The polymer solutions were all gamma (γ)-irradiated.

B. Preparation of Octreotide Acetate+Citric Acid Mixture

Octreotide acetate and citric acid mixture was prepared by dissolving 3.5002 g of octreotide acetate, and 0.6604 g citric acid into 30 mL HPLC grade water. The solution was stirred until all solids were in solution. The weights used above were derived from a calculated 1:1 ratio of octreotide to citric acid. The solution was divided into five separate scintillation vials, frozen at −86 C for one hour, then lyophilized for two days.

C. Preparation of A-B Syringes

Seven syringe pairs, 960 mg of formulation in each pair, were used for each group. The B syringes (male syringes) were prepared by pipetting certain amount of octreotide stock solution into 1.25 mL BD syringes, followed by lyophilization for 24 hours. A syringes (female syringes) were prepared by weighing polymer solution into 1 mL female syringes.

### Experimental Design

In this 99-day study, three ATRIGEL®/Octreotide formulations were tested in one hundred and thirty-five male rats with forty-five rats per treatment group. On Day 0, each rat received one 100 μL (approximate) SC injection of formulation containing approximately 12 mg, 13.5 mg or 15 mg octreotide in the DT region. On Days 1, 4, 7, 14, 28, 60, 75, 90, and 99, five rats per group were anesthetized and bled (up to 5 mL) via cardiac puncture. Following blood collection, each animal was euthanized by CO₂ and TAs retrieved for subsequent RP-HPLC analysis to determine their octreotide content. Macroscopic SC tissue reaction, relative to each TA, was evaluated by gross examination of the implants and the surrounding tissue. Plasma was analyzed for octreotide content by LC/MS/MS at ABC Laboratories (Columbia, Mo.).

| | | | Drug Dosage | | |
|---|---|---|---|---|---|
| | | Test | of Octreotide | | |
| | Number/ | Article/ | acetate + | Formulation | Termination |
| Group | Gender | Route | citric acid | Volume | Time Point |
| I | 45/M | 1/SC | 12 mg | 100 μL | Days 1, |
| | | | | | 4, 7, 14, |
| | | | | | 28, 60, 75, |
| | | | | | 90, and 99 |
| II | 45/M | 2/SC | 13.5 mg | 100 μL | Days 1, |
| | | | | | 4, 7, 14, |
| | | | | | 28, 60, 75, |
| | | | | | 90, and 99 |
| III | 45/M | 3/SC | 15 mg | 100 μL | Days 1, |
| | | | | | 4, 7, 14, |
| | | | | | 28, 60, 75, |
| | | | | | 90, and 99 |

### Experimental Procedure

The in-life portion of the study lasted 99 days. A dose of 12, 13.5 or 15 mg octreotide was used. While under general isoflurane anesthesia, each rat was placed in sternal recumbency, its DT region shaved, and the injection site wiped with isopropanol. Each animal was administered a single 100 μL SC injection of appropriate TA in the DT region. During the course of the study the animals were observed for signs of overt toxicity and for any existing abnormalities, including redness, bleeding, swelling, discharge, bruising, and TA extrusion. Body weights were taken at administration and at termination.

On Days 1, 4, 7, 14, 28, 60, 75, 90, and 99, five rats per group were anesthetized and bled (up to 5 mL) via cardiac puncture. Following blood collection, each rat was euthanized with CO₂ and implants recovered. Representative photographs of the test sites were taken and precipitation characteristics of the implants were documented. Implants were placed in dry, labeled vials. Mean and standard deviation were used in this study.

### Protocol Modifications

There were two protocol modifications during the course of this study. In the protocol, the procedure was that on Days 14, 28, 60, and 90, an ˜2 cm×2 cm area of skin surrounding the implant was to be collected for histopathological evaluation. Skin tissue samples surrounding the implants were inadvertently not collected on Day 14 as stated in the final protocol. Days 28, 60 and 90 tissue samples were collected and processed. In the second modification, the histology slides were not analyzed for histopathology. The implant retrieval procedure removed the fatty tissue surrounding the implant, thus the pathologist was not able to analyze the skin samples due to the disturbance of the fatty tissue. One clarification is that all test articles were formulated with an inherent viscosity of 0.27 rather than 0.25, as stated in the final protocol. There were no adverse affects to the study as a result of these changes.

### Results and Discussion

Overt toxicity observations noted during the course of the study were unremarkable. Test site observations noted few instances of redness and bruising at TA sites in all groups through Day 2.

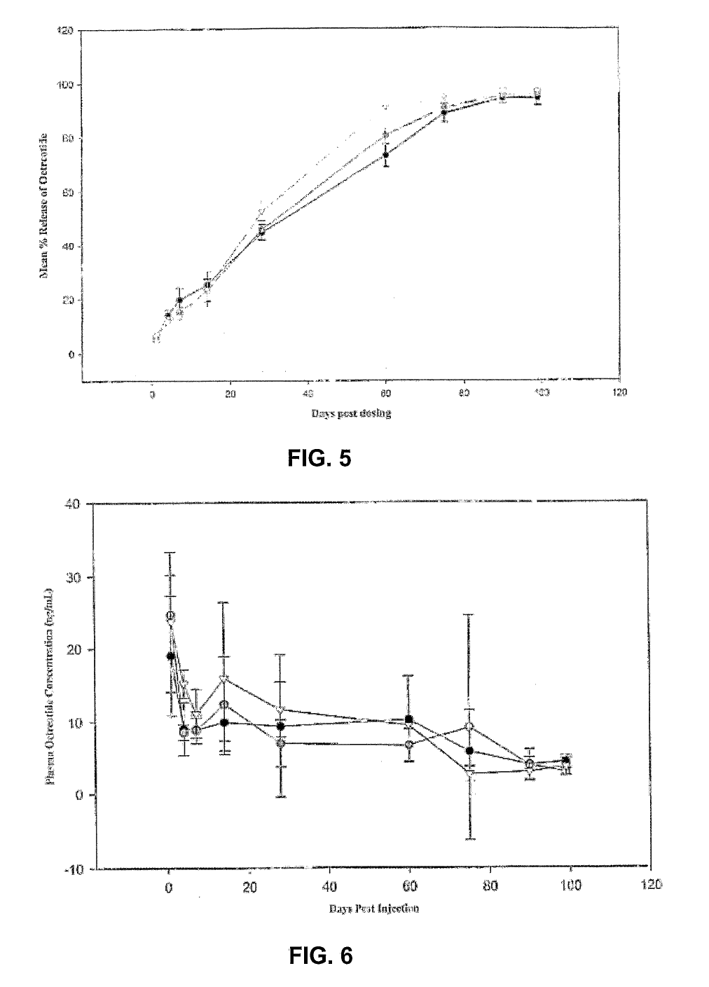

Table 3-1 illustrates the percentage of octreotide released from each formulation. The initial burst (Day 1 release of octreotide) was 6.1%, 6.3%, and 6.9% for Groups I, II and II, respectively. All of the formulations released approximately 95% to 97% octreotide by Day 99. The mean release of octreotide for all formulations is depicted in FIG. 5.

TABLE 3-1
Percent of Octreotide Released from Three ATRIGEL ®/Octreotide
Formulations Following Subcutaneous Injection in Rats

| Test | Time | Mean % | Standard |
|---|---|---|---|
| Article | Point | Released | Deviation |
| Group I: 12% Octreotide acetate + | Day 1 | 6.1 | 1.0 |
| citric acid in 50% 85/15 PLGH (InV | Day 4 | 14.0 | 1.7 |
| 0.36)/50% NMP, irradiated | Day 7 | 19.6 | 4.5 |
| | Day 14 | 25.2 | 2.3 |
| | Day 28 | 44.8 | 2.9 |
| | Day 60 | 73.0 | 4.2 |
| | Day 75 | 88.5 | 3.4 |
| | Day 90 | 94.2 | 1.9 |
| | Day 99 | 94.3 | 2.7 |
| Group II: 13.5% Octreotide acetate + | Day 1 | 6.3 | 1.7 |
| citric acid in 50% 85/15 PLGH (InV | Day 4 | 11.9 | 1.3 |
| 0.27)/50% NMP, irradiated | Day 7 | 14.9 | 2.1 |
| | Day 14 | 23.2 | 4.0 |
| | Day 28 | 46.0 | 3.5 |
| | Day 60 | 80.3 | 3.2 |
| | Day 75 | 90.5 | 1.3 |
| | Day 90 | 95.4 | 1.5 |
| | Day 99 | 95.9 | 0.7 |
| Group III: 15% Octreotide acetate + | Day 1 | 6.9 | 1.7 |
| citric acid in 50% 85/15 PLGH (InV | Day 4 | 11.5 | 2.1 |
| 0.27)/50% NMP, irradiated | Day 7 | 13.6 | 2.5 |
| | Day 14 | 23.3 | 6.8 |
| | Day 28 | 52.6 | 4.3 |
| | Day 60 | 90.6 | 0.8 |
| | Day 75 | 94.4 | 1.2 |
| | Day 90 | 97.3 | 0.5 |
| | Day 99 | 97.2 | 0.4 |

Table 3-2 summarizes the plasma octreotide levels for all groups. The mean plasma levels were depicted in FIG. 6. All groups reached Cₘₐₓ at Day 1. The Cₘₐₓ of Groups I through III are 19.0±8.2 ng/mL, 24.6±5.6 ng/mL, and 23.7±9.6 ng/mL respectively. The area under the curve (AUC_{day 0-99}) of Groups I through III are 818.7 ng-day/mL, 654.65 and 893.44 ng-day/mL, respectively. The dose normalized AUC_{day 0-99} for Groups I to III are 65.06, 53.22 and 54.51 ng·day/mL, respectively.

TABLE 3-2
Octreotide Plasma Concentrations in Rats Following Subcutaneous
Injection of ATRIGEL ®/Octreotide Formulations

| | | Mean | |
|---|---|---|---|
| | | Octreotide | |
| Test | Time | in plasma | Standard |
| Article | Point | (ng/mL) | Deviation |
| Group I: 12% Octreotide acetate + | Day 1 | 19.0 | 8.2 |
| citric acid in 50% 85/15 PLGH (InV | Day 4 | 9.0 | 3.7 |
| 0.27)/50% NMP, irradiated | Day 7 | 8.9 | 2.0 |
| | Day 14 | 9.9 | 2.6 |
| | Day 28 | 9.3 | 9.8 |
| | Day 60 | 10.2 | 5.9 |
| | Day 75 | 5.7 | 5.8 |
| | Day 90 | 3.9 | 1.0 |
| | Day 99 | 4.3 | 0.8 |
| Group II: 13.5% Octreotide acetate + | Day 1 | 24.6 | 5.6 |
| citric acid in 50% 85/15 PLGH (InV | Day 4 | 8.5 | 1.1 |
| 0.27)/50% NMP, irradiated | Day 7 | 8.8 | 1.8 |
| | Day 14 | 12.4 | 6.5 |
| | Day 28 | 6.9 | 3.3 |
| | Day 60 | 5.8 | 7.6 |
| | Day 75 | 9.1 | 15.4 |
| | Day 90 | 3.8 | 2.2 |
| | Day 99 | 2.9 | 0.4 |
| Group III: 15% Octreotide acetate + | Day 1 | 23.7 | 9.6 |
| citric acid in 50% 85/15 PLGH (InV | Day 4 | 15.2 | 1.9 |
| 0.27)/50% NMP, irradiated | Day 7 | 11.1 | 3.4 |
| | Day 14 | 15.9 | 10.5 |
| | Day 28 | 11.6 | 3.8 |
| | Day 60 | 4.6 | 4.4 |
| | Day 75 | 2.6 | 1.0 |
| | Day 90 | 2.9 | 1.1 |
| | Day 99 | 3.6 | 1.2 |

### Conclusions

This study compared the release characteristics of formulations containing three different concentrations (12, 13.5, and 15% w/w) of octreotide acetate/citric acid in an ATRIGEL® system prepared from a slightly higher molecular weight 85/15 PLGH obtained from a different polymer supplier. The implant retrieval data showed that all of the formulations had a very low initial burst of octreotide at Day 1 with a range of 6.1% to 6.9%. These are the lowest initial burst values obtained to date with any ATRIGEL® formulations containing the octreotide acetate/citric acid mixture. Although there is very little difference among the three formulations, the trend appears to be that the higher drug concentrations give a slightly higher initial burst. There is also little difference in the cumulative release rates of the three formulations until around Day 28. From Day 28 to Day 60, the formulations with the higher drug mixture appear to give a faster overall release of drug, especially the formulation in Group III with 15% octreotide acetate/citrate. However, at Day 60, the cumulative release of this formulation has reached 90.6%, and the rate began to decrease. These data suggest that the higher concentration of drug mixture may lead to a more porous substrate as the drug dissolves and releases from the implant. The more porous implant should give a faster release of remaining drug until about 90% of the drug has been released.

The data from the plasma analyses showed that the maximum octreotide plasma concentrations (Cₘₐₓ) were achieved by Day 1 for all three formulations. However, because the initial drug burst was very low for the three formulations, the Cₘₐₓ values for all three formulations were the lowest concentrations observed thus far for any ATRIGEL®/Octreotide formulations. The values ranged from 19.0±8.2 ng/mL to 24.6±5.6 ng/mL, with no major difference among the three formulations. Plasma octreotide levels had decreased by Day 4 and remained at a relatively steady level to Day 60. After that time, all three formulations showed a gradual decrease in octreotide plasma concentrations through Day 99 as expected from the decline in release rates noted for the implant retrieval data.

Tissue irritation as determined by macroscopic evaluation was none to minimal for all groups. Most of the irritation observed was on Day 1 with some isolated instances through Day 14.

In conclusion, the results of this study show that ATRIGEL® formulations containing 12%-15% of an octreotide acetate/citric acid mixture in 50% 85/15 PLGH (0.27 In) and 50% NMP solution give low initial drug burst (6-7%) and sustained release of octreotide out to 99 days. The higher drug mixture formulations appear to give the faster overall release of drug. However, all formulations with the slightly higher molecular weight 85/15 PLGH polymer from Absorbable Polymers Technologies, Inc. give highly desirable release characteristics.

### Example 4

### A 90 Day Pharmacokinetics and Pharmacodynamics Study of ATRIGEL®/Octreotide After a Single Subcutaneous Injection in Male Rabbits

### Summary

The purpose of this study was to determine the drug release profile, pharmacokinetics (PK) and pharmacodynamics (PD) of a 3-month ATRIGEL®/Octreotide formulation following subcutaneous (SC) injection in rabbits. The primary objective of this study was to determine the 90-day PK and PD of octreotide in rabbit plasma after a single injection of an ATRIGEL®/Octreotide formulations. A secondary objective was to determine the release kinetics of ATRIGEL®/Octreotide utilizing implant retrieval and subsequent reversed phase-high performance liquid chromatography (RP-HPLC). The tertiary objective was to determine the Insulin-Like Growth Factor-1 (IGF-1) levels at various time points from rabbit serum. An additional objective was to evaluate test sites macroscopically for tissue reactions and test article (TA) characteristics.

In this 90-day study, one ATRIGEL®/Octreotide formulation was tested in five male rabbits. On Day 0, each rabbit received one 0.86 mL (approximate) SC injection of formulation containing approximately 90 mg octreotide in the dorsal thoracic (DT) region. On Days −7, −2, 0, (pre-dose), 0.3, 1, 7, 14, 21, 28, 43, 49, 59, and 76, five rabbits were bled (up to 6 mL) via central ear artery or lateral ear vein. Serum and plasma was derived and analyzed for IGF-1 analysis and octreotide analysis, respectively. On Day 90, each animal was anesthetized and bled via cardiac puncture. Following blood collection, each animal was euthanized and TAs retrieved for subsequent RP-HPLC analysis to determine octreotide content. Macroscopic SC tissue reaction, relative to each TA, was evaluated by gross examination of the implants and surrounding tissue. Plasma octreotide levels were analyzed by Liquid Chromatography-Mass Spectrometry/Mass Spectrometry (LC-MS/MS) at ABC Laboratories (Columbia, Mo.). Serum IGF-1 levels were measured by a competitive binding Radioimmunoassay (RIA) at Esoterix Center for Clinical Trials (Calabasas Hills, Calif.).

Maximum mean plasma octreotide levels (Cₘₐₓ) in rabbits reached 113.4±165.2 ng/mL at 24 hours post dosing. Octreotide plasma levels remained at a relatively steady state above 17.5±3.6 ng/mL until Day 76. By Day 90, plasma octreotide levels decreased to 2.5±0.2 ng/mL. A substantial suppression of IGF-1 levels (35.6%) was observed from pre-dose (109.7±33.7 ng/mL) to 7 hours post-dose (70.6±34.3 ng/mL). The lowest IGF-1 level of 55.2±20.3 ng/mL was seen at Day 42. There were no gross observations of any tissue irritation during the course of the study. In summary, rabbit plasma octreotide levels remained higher than therapeutic levels of 0.3 ng/mL (Marbach et al., From Somatostatin to Sandostatin: Pharmacodynamics and Pharmacoinetics, Metabolism, Vol 41, No. 9, Suppl. 2 (September), 1992: pp 7-10) throughout the study. Correspondingly, IGF-1 levels were suppressed, indicating efficacy at this dosage.

### Introduction

Octreotide is a synthetic, eight amino acid peptide marketed by Novartis. The primary indication for octreotide is for the treatment of acromegaly caused by hypersecretion of growth hormone, and is indicated for the symptomatic control of metastatic carcinoid and vasoactive intestinal peptide-secreting tumors. The current clinical formulations are administered as subcutaneous daily injections (Sandostatin®), or as a single one-month sustained-release intramuscular depot (Sandostatin LAR® [\ddot{L}ong Äcting \ddot{R}elease]). The one-month depot product is a microparticulate formulation in which the drug is encapsulated in microspheres that are prepared from glucose and poly(DL-lactide-co-glycolide) [PLG] polymers.

The ATRIGEL® drug delivery system is a biodegradable polymeric delivery system that can be injected as a liquid. Upon injection of the formulation, the polymer solidifies encapsulating the drug. As the process of biodegradation begins, the drug is slowly released. The release rate of drugs from this type of delivery system can be controlled by the type and molecular weight of the polymer, and drug load of the constituted product. Therefore the system can be tailored to meet the needs of the patient.

### Materials and Methods

This was a single dose in vivo study designed to determine the 90-day release kinetics, PK and PD of one ATRIGEL®/Octreotide formulation administered SC in rabbits. The inherent viscosity (InV) is in units of dL/g and the formulation was mixed by A/B mixing.

Test Article Identification: 90 mg Octreotide in (50% 85/15 PLGH (InV 0.27)/50% NMP); Control Article: No controls were used in this study.

### Manufacturer Information

| Substance | Manufacturer | Lot# |
|---|---|---|
| Octreotide acetate | Bachem | 110702-003 |
| Citric Acid | Fisher | 001852 |
| NMP | | TN-013102-000 |
| 85/15 PLGH (InV 0.27) | APT | A140-13 |

### Formulation Preparation:

Polymer stock solution was prepared by weighing a known amount of polymer solid into a 20 mL scintillation vial. A known amount of NMP was added to the polymer and the mixture placed on a jar mill. The vials were mixed at least overnight, producing a visually clear polymer solution. The polymer solution was γ-irradiated.

A. Preparation of Octreotide Acetate+Citric Acid Mixture

Octreotide acetate and citric acid mixture was prepared by dissolving 1.6000 g of octreotide acetate, and 0.3014 g citric acid into 16.32 mL HPLC grade water. The solution was stirred until all solids were in solution. The weights used above were derived from a calculated 1:1 ratio of octreotide to citric acid. The solution was divided into 4 separate scintillation vials, frozen at −86 C for one hour, then lyophilized for two days.

B. Preparation of A-B Syringes

Ten syringe pairs, 899 mg formulations in each pair, were prepared. Syringe pairs 1-5 were used to inject the rabbits. The B syringes (male syringes) were prepared by weighing 1.000 g octreotide stock solution (13.5% octreotide drug powder) into 3 mL syringes, followed by lyophilization for 48 hours. A syringes (female syringes) were prepared by weighing ˜764.3 mg polymer solution into 3 mL female syringes.

### Experimental Design

In this 90-day study, one ATRIGEL®/Octreotide formulation was tested in five male rabbits. On Day 0, each rabbit received one 0.86 mL (approximate) SC injection of formulation containing approximately 90 mg octreotide in the DT region. On Days −7, −2, 0, (pre-dose), 0.3, 1, 7, 14, 21, 28, 43, 49, 59, and 76, five rabbits were bled (up to 6 mL) via central ear artery or lateral ear vein. Serum and plasma was derived and analyzed for IGF-1 analysis and octreotide analysis, respectively. On Day 90, each animal was anesthetized and bled via cardiac puncture. Following blood collection, each animal was euthanized and TAs retrieved for subsequent RP-HPLC analysis to determine octreotide content. Macroscopic SC tissue reaction, relative to each TA, was evaluated by gross examination of the implants and the surrounding tissue. Plasma octreotide levels were analyzed by LC/MS/MS at ABC Laboratories (Columbia, Mo.). Serum IGF-1 levels were measured by a competitive binding RIA at Esoterix Center for Clinical Trials (Calabasas Hills, Calif.).

| | | Test | Drug | | Blood |
|---|---|---|---|---|---|
| | Number/ | Article/ | Dosage | Formulation | Collection |
| Group | Gender | Route | of Octreotide | Volume | Time Point |
| I | 5/M | 1/SC | 90 mg | 0.86 mL | Days −7, −2, |
| | | | | | 0 (pre-dose), |
| | | | | | 0.3, 1, 7, 14, |
| | | | | | 21, 28, 43, |
| | | | | | 49, 59, |
| | | | | | 76, and 90 |

### Experimental Procedure

The in-life portion of the study lasted 90 days. A dose of 90 mg octreotide was used. While under general isoflurane anesthesia, each rabbit was placed in sternal recumbency, its DT region shaved, and the injection site wiped with isopropanol. Each animal was administered a single 0.86 mL (approximate) SC injection of ATRIGEL®/Octreotide in the DT region. During the course of the study the animals were observed for signs of overt toxicity. On Days 0-7, 14, 21, 28, and 42, the animals were observed for any existing abnormalities, including redness, bleeding, swelling, discharge, bruising, and TA extrusion. Body weights were taken at all blood collection time points (except Day 0.3) and at termination.

Rabbits were fasted approximately 12 hours prior to blood collection. Prior to immediate blood collection, rabbits were sedated by a SC 0.2 mg/kg dose of acepromazine maleate in the central cranial dorsal region. On Days −7, −2, 0 (pre-dose), 0.3, 1, 7, 14, 21, 28, 43, 49, 59, and 76, blood was collected from the central ear artery or lateral ear vein into serum separator tubes (˜2 mLs blood) and sodium heparin tubes (˜4 mLs blood). Serum was derived, frozen, and then shipped to Esoterix Endocrinology for IGF-1 analysis. Plasma was derived, frozen, and then shipped to ABC Laboratories for octreotide analysis. On Day 90, each rabbit was anesthetized and bled via cardiac puncture. Following blood collection, each rat was euthanized and weighed and implants recovered. Representative photographs of the test sites were taken and precipitation characteristics of the implants were documented. Implants were placed in dry, labeled vials. Skin tissue samples were collected from the area surrounding the implant. Mean and standard deviation were used in this study.

### Results and Discussion

Overt toxicity and test site observations noted during the course of the study were unremarkable. The targeted injection volume was 860 μL (0.86 mL) of formulation. The injection weights ranged from 759.0 μL to 782.7 μL. Table 4-1 reflects the implant retrieval data on Day 90. The data showed that 1.7% of the octreotide dosed to rabbits remained in the depot 90 days post dosing.

TABLE 4-1
Percent of Octreotide Remaining in Depot 90 Days After
Subcutaneous Injection in Rabbits

| | Time | Octreotide | Mean | |
|---|---|---|---|---|
| Test | Point | Remaining | Octreotide | Standard |
| Article | (Day) | (%) | Remaining % | Deviation |
| Group I: 15% Octreotide | Day | 0.80 | 1.7 | 1.2 |
| acetate + citric acid in | 90 | 2.90 | | |
| 50% 85/15PLGH | | 1.1 | | |
| (InV 0.27)/50% NMP | | 3.1 | | |
| | | 0.8 | | |

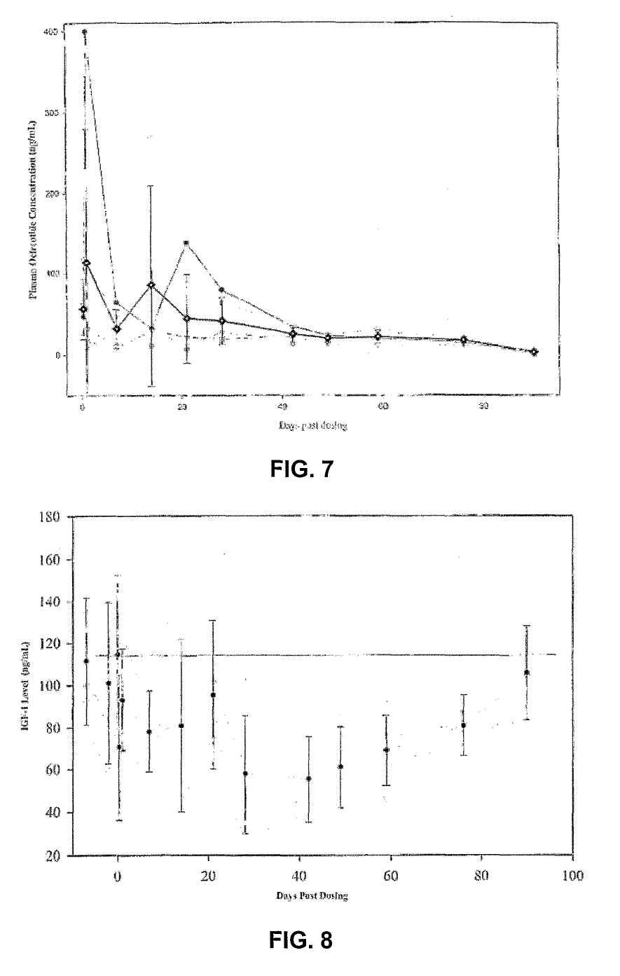

Table 4-2 contains the plasma octreotide concentrations for the five rabbits that received approximately 90 mg octreotide. Octreotide plasma levels that were assayed at below the quantifiable limit (BQL: 0.5 ng/mL octreotide) were assigned an octreotide concentration equal to 0 ng/mL. The mean plasma concentrations reached a maximum of 113.4±165.2 ng/mL at 24 hours post dosing, then remained above 17.5±3.6 ng/mL until Day 76. Plasma octreotide levels dropped to 2.5±2.2 ng/mL at Day 90. The area under the curve (AUC₀₋₉₀) was 2518.6 ng day/mL, and the dose normalized AUC₀₋₉₀ was 27.98 ng·day/mg·mL. The individual and mean plasma concentrations are depicted in FIG. 7.

TABLE 4-2
Pharmacokinetic Profile of a 3-month ATRIGEL ®/Octreotide
Formulation Following Subcutaneous Injection in Rabbits

| | | Mean | |
|---|---|---|---|
| | | Plasma | |
| | | Octreotide | |
| Test | Time | Levels | Standard |
| Article | Point | (ng/mL) | Deviation |
| Group I: 15% Octreotide acetate + citric | 0 | 0.0 | 0.0 |
| acid in 50% 85/15 PLGH (InV 0.27)/ | 0.3 | 55.8 | 36.9 |
| 50% NMP | 1 | 113.4 | 165.2 |
| | 7 | 31.9 | 23.5 |
| | 14 | 85.4 | 123.4 |
| | 21 | 44.1 | 54.3 |
| | 28 | 40.6 | 28.4 |
| | 42 | 25.2 | 9.9 |
| | 49 | 20.0 | 4.6 |
| | 59 | 21.3 | 7.9 |
| | 76 | 17.5 | 3.6 |
| | 90 | 2.5 | 2.2 |

The individual and mean serum IGF-1 data are listed in Table 4-3 and graphically depicted in FIG. 8. The pre-dose IGF-1 levels were 111.6±30.2 (Day −7), 101.0±38.5 (Day −2) and 114.6±38.1 ng/mL (Day 0). The mean pre-dose IGF-1 level is 109.7±33.69 ng/mL. Mean IGF-1 levels decreased to 70.6±34.3 ng/mL at 7 hours post dosing, and reached the lowest IGF-1 level (55.2±20.3 ng/mL) at Day 42. The IGF-1 levels remained below 80.8±14.7 ng/mL until Day 76 (26.3% suppression versus pre-dose level), followed by a slow increase to 105.8±22.1 ng/mL at Day 90. Overall, serum IGF-1 levels dropped considerably 7 hours post dosing, and remained suppressed throughout the study.

TABLE 4-3
Pharmacodynamic Profile of a 3-month ATRIGEL ®/Octreotide
Formulation Following Subcutaneous Injection in Rabbits

| | | Mean | |
|---|---|---|---|
| | Time | Serum IGF-1 | |
| Test | Point | Levels | Standard |
| Article | (Day) | (ng/mL) | Deviation |
| Group I: 15% Octreotide acetate + citric | −7 | 111.6 | 30.2 |
| acid in 50% 85/15 PLGH (InV 0.27)/ | −2 | 101.0 | 38.5 |
| 50% NMP | 0 | 114.6 | 38.1 |
| | 0.3 | 70.6 | 34.3 |
| | 1 | 93.2 | 24.2 |
| | 7 | 78.0 | 19.3 |
| | 14 | 81.0 | 41.1 |
| | 21 | 95.4 | 35.4 |
| | 28 | 57.8 | 27.9 |
| | 42 | 55.2 | 20.3 |
| | 49 | 61.0 | 19.3 |
| | 59 | 68.8 | 16.8 |
| | 76 | 80.8 | 14.7 |
| | 90 | 105.8 | 22.1 |

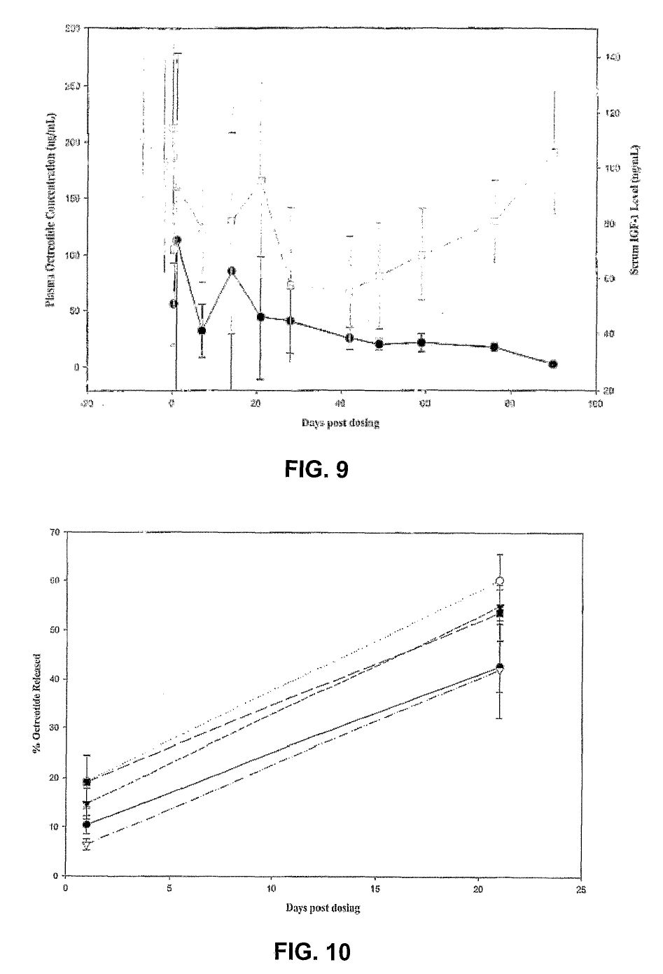

FIG. 9 illustrates the correlation between the mean rabbit PK and PD data. Throughout the 90-day study, rabbit plasma octreotide levels remained higher than 0.3 ng/mL, (therapeutic level). Correspondingly, IGF-1 levels were suppressed, indicating efficacy at this dosage.

### Conclusions

The PK data showed that there was an initial burst of drug from the formulation followed by sustained release for 90 days. The maximum (113.4±165.2) octreotide plasma concentrations (Cₘₐₓ) were recorded on Day 1 for all animals. From Day 7 to Day 76, the octreotide plasma concentrations achieved a relatively stable level ranging from 17.5 to 31.9 ng/mL. However, by Day 90, the octreotide plasma concentration had decreased to a mean value of 2.5±2.2 ng/mL, indicating a possible reduction in the release rate.

The implant retrieval data showed that only 1.7% of octreotide dosed to the rabbits remained in the depot 90 days post-dosing. This low level of residual drug in the implants correlates with the lower plasma levels of octreotide obtained toward the end of the study.

The data for the individual and mean serum IGF-1 concentrations show a substantial suppression of IGF-1 levels within the first seven days that gradually increased until the maximum suppression was achieved at Day 28. However, by Day 56, the IGF-1 levels slowly began to increase until at Day 90 they had returned to pre-dose levels. There appeared to be a correlation between the mean octreotide concentrations (PK) and the IGF-1 levels (PD) for the study. The serum concentration of IGF-1 decreased quickly after administration of the formulation where high plasma levels of octreotide were obtained. Later on in the study where the plasma concentration of octreotide decreased, the level of IGF-1 started to increase, an indication of a good correlation between the two parameters.

There was no tissue irritation as determined by macroscopic evaluation at Day 90. In addition, there were no gross observations of tissue irritation during the course of the study.

In conclusion, the results of this study show that an ATRIGEL® formulation containing 12% of an octreotide acetate/citric acid mixture in 50% NMP 85/15 PLGH (0.27 InV) and 50% NMP solution gives sustained release of octreotide for 90 days when injected SC in rabbits. The IGF-1 data shows that the octreotide released is biologically active as the post-dosing levels are substantially reduced compared to pre-dosing concentrations. However, the return of the IGF-1 levels to pre-dose values by Day 90 indicates that higher concentrations of octreotide may be required at the later times to maintain efficacy in the rabbit model.

### Example 5

### A 21-Day Release and Absorption Kinetics Study of ATRIGEL®/Octreotide Formulations Following a Single Subcutaneous Injection in Male Rats

### Summary

The purpose of this study was to evaluate the Day 1 and Day 21 release kinetics of five ATRIGEL®/Octreotide formulations containing various polymers and solvents administered subcutaneously (SC) in rats. The primary objective was to determine the Day 1 and Day 21 release profile of five modified ATRIGEL®/Octreotide formulations utilizing implant retrieval and subsequent reversed phase high performance liquid chromatography (RP-HPLC). The secondary objective was to evaluate test sites macroscopically for tissue reactions and test article (TA) characteristics.

In this 21-Day study, five ATRIGEL® formulations were tested in fifty male rats, ten rats per treatment group. On Day 0, each rat received one 100 μL (approximate) SC injection of formulation containing approximately 15 mg octreotide in the dorsal thoracic (DT) region. On Days 1, and 21, five rats per group were anesthetized and bled (up to 5 mL) via cardiac puncture. Plasma was derived and analyzed for octreotide content by ABC Laboratories. Following blood collection, rats were euthanized by CO₂ and TAs retrieved for subsequent RP-HPLC analysis to determine their octreotide content. Plasma Macroscopic SC tissue reaction, relative to each TA, was evaluated by gross examination of the implants and the surrounding tissue.

Five ATRIGEL® formulations were loaded with 15% octreotide drug powder and employed 85/15 PLGH (InV 0.25 to 0.28) in the delivery system. The raw polymer used in Groups I, II, III, and V was purchased from Alkermes and the Group IV polymer was purchased from Adsorbable Polymer Technologie (APT). Groups II [(InV 0.25) in 50% NMP+1.4% CH₂Cl₂], III [(InV 0.28) in 50% NMP] and V [(InV 0.25) in 50% NMP] demonstrated similar octreotide release profiles at Day 1 and Day 21. The addition of methylene chloride to the Group II formulation and slightly higher inherent viscosity of Group III did not reduce the initial burst nor change the rate of octreotide release as evidenced in Table 5. Groups I and IV released approximately 10% less octreotide at Day 1 and Day 21 than the other groups. The data suggests that purification (dissolution and precipitation) of the polymer (Group I) reduces the release rate of octreotide considerably. Tissue reaction to the TAs were unremarkable.

### Introduction

Octreotide is a synthetic, eight amino acid peptide marketed by Novartis. The primary indication for octreotide is for the treatment of acromegaly caused by hypersecretion of growth hormone, and is indicated for the symptomatic control of metastatic carcinoid and vasoactive intestinal peptide-secreting tumors. The current clinical formulations are administered as subcutaneous daily injections (Sandostating), or as a single one-month sustained-release intramuscular depot (Sandostatin LAR® [\ddot{L}ong Äcting \ddot{R}elease]). The one-month depot product is a microparticulate formulation in which the drug is encapsulated in microspheres that are prepared from glucose and poly(DL-lactide-co-glycolide) [PLG] polymers.

The ATRIGEL® drug delivery system is a biodegradable polymeric delivery system that can be injected as a liquid. Upon injection of the formulation, the polymer solidifies encapsulating the drug. As the process of biodegradation begins, the drug is slowly released. The release rate of drugs from this type of delivery system can be controlled by the type and molecular weight of the polymer and drug load of the constituted product. Therefore the system can be tailored to meet the needs of the patient.

This was a single dose in vivo study designed to determine the Day 1 and Day 21 release kinetics of octreotide delivered from five modified ATRIGEL® formulations injected SC into rats. All percentages are weight to weight (w/w) and all inherent viscosities (InV) are in units of dL/g.

### Test Article Identification

1. 15% Octreotide acetate+citric acid (OTCA) in re-precipitated (or purified) 50% Alkermes 85/15 PLGH (InV 0.25) in 50% NMP.
2. 15% OTCA in 50% Alkermes 85/15 PLGH (InV 0.25) in 50% NMP+1.4% CH₂Cl₂.
3. 15% OTCA in 50% Alkermes 85/15 PLGH (InV 0.28) in 50% NMP.
4. 15% OTCA in 50% APT 85/15 PLGH (InV 0.27) in 50% NMP.
5. 15% OTCA in 50% Alkermes 85/15 PLGH (InV 0.25) in 50% NMP
   Control Article There were no controls used in this study.

### Manufacturer Information:

| Substance | Manufacturer | Lot# |
|---|---|---|
| Octreotide acetate | Bachem | 110702-003 |
| Citric Acid | Fisher | 006630 |
| NMP | | TN-013102-000 |
| 85/15 PLGH (InV 0.27) | APT | A140-13 |
| 85/15 PLGH (InV 0.25) | Alkermes | 02-012-39 |
| 85/15 PLGH (InV 0.28) | Alkermes | 00-141-19 |
| Methylene Chloride | Fisher | RDC#02428D |
| Methanol | | RDC# 03734 |

### Formulation Preparation

A. Preparation of Purified 85/15 PLGH (InV 0.25)

Approximately 48.25 g of 85/15 PLGH (InV 0.25), purchased from Alkermes was weighed into a glass jar and 100 mL of methylene chloride was added. The jar was placed on a jar mill overnight until a visually clear polymer solution was produced. The polymer solution was slowly added into 500 mL of methanol while stirring continuously to precipitate the polymer. The precipitated polymer was rinsed twice with 200 mL and 100 mL of methanol, respectively. The purified polymer was put in a vacuum oven at room temperature for one day to remove solvents. Approximately 40.29 g of polymer was recovered.

B. Preparation of Polymer Solution

Polymer stock solutions were prepared by weighing a known amount of each polymer solid into individual 20 mL scintillation vials. A known amount of NMP was added to each polymer and the mixture placed on a jar mill. For Group II, 1.4% (w/w to total gel) methylene chloride was added to the solution. The vials were mixed overnight or until a visually clear polymer solution was produced. The polymer solutions were all γ-irradiated.

C. Preparation of Octreotide Acetate+Citric Acid Mixture

An octreotide acetate and citric acid mixture was prepared by dissolving 4.0002 g of octreotide acetate and 0.7550 g citric acid into 30 mL HPLC grade water. The solution was stirred until all solids were in solution. The weights used above were derived from a calculated 1:1 ratio of octreotide to citric acid. The solution was divided into 5 separate scintillation vials, frozen at −86° C. for one hour, then lyophilized for two days.

D. Preparation of A-B Syringes

B syringes (male syringes) were prepared by pipetting 500 mg of octreotide stock solution into 1.25 mL BD syringes then lyophilized for 24 hours. The stock solution was prepared by dissolving 1.3508 g octreotide+citric acid mixture in 4.6537 g HPLC grade water, creating a 22.5% (w/w) stock solution. The A syringes (female syringes) were prepared by weighing 637.5 mg polymer solution into 1 mL female syringes.

### Experimental Design

In this 21-Day study, five ATRIGEL® formulations were tested in fifty male rats, ten rats per treatment group. On Day 0, each rat received one 100 μL (approximate) SC injection of formulation containing approximately 15 mg octreotide in the DT region. On Days 1, and 21, five rats per group were anesthetized and bled (up to 5 mL) via cardiac puncture. Plasma was derived and analyzed for octreotide content by ABC Laboratories. Following blood collection, rats were euthanized by CO₂ and TAs retrieved for subsequent RP-HPLC analysis to determine their octreotide content. Plasma Macroscopic SC tissue reaction, relative to each TA, was evaluated by gross examination of the implants and the surrounding tissue.

| | | Test | Drug | | |
|---|---|---|---|---|---|
| | Number/ | Article/ | Dosage | Formulation | Termination |
| Group | Gender | Route | of OTCA | Volume | Time Point |
| I | 10/M | 1/SC | 15 mg | 100 μL | Days 1, and 21 |
| II | 10/M | 2/SC | 15 mg | 100 μL | Days 1, and 21 |
| III | 10/M | 3/SC | 15 mg | 100 μL | Days 1, and 21 |
| IV | 10/M | 4/SC | 15 mg | 100 μL | Days 1, and 21 |
| V | 10/M | 5/SC | 15 mg | 100 μL | Days 1, and 21 |

### Experimental Procedure

The in-life portion of the study lasted 21 days. A dose of 15.0 mg OTCA was used. While under general isoflurane anesthesia, each rat was placed in sternal recumbency, its DT region shaved, and the injection site wiped with isopropanol. Each animal was administered a single 100 μL SC injection of appropriate TA in the DT region via 19-gauge thin-wall needle. During the course of the study the animals were observed for signs of overt toxicity. On Days 0-7, 14 and 21, animals were observed for any existing abnormalities, including redness, bleeding, swelling, discharge, bruising, and TA extrusion. Body weights were taken at administration and at termination.

On Days 1, and 21, five rats per group were anesthetized and bled via cardiac puncture. Following blood collection, animals were euthanized with CO₂ and implants recovered. Representative photographs of the test sites were taken and precipitation characteristics of the implants were documented. Implants were placed in dry, labeled vials.

Mean and standard deviation were used in this study. There were no protocol modifications during the course of this study.

### Results and Discussion

Overt toxicity and recorded during the course of the study were unremarkable. Test site observations noted scabbing at the test site of Group II and V animals from Days 3 through 6.

The targeted dose for the study was 100 μL (100 mg) of formulation. The mean injection weights from each group were: 91.8±17.9 mg for Group I, 101.9±10.3 mg for Group II, 90.7±21.7 mg for Group III, 99.8±23.4 mg for Group IV, and 83.1±17.1 mg for Group V. Since this study was an implant retrieval study, and the injection weights were recorded, the amount of each TA injected could have a wide range without adversely affecting the outcome of the study. Table 5-1 and FIG. 10 illustrate the percentage of octreotide released from each formulation at Day 1 and Day 21.

TABLE 5-1
Percent of Octreotide Released Following SC Injection in Rats

| Test | Time | Mean % | Standard |
|---|---|---|---|
| Article | Point | Released | Deviation |
| Group I: 15% Octreotide acetate + | Day 1 | 10.5 | 1.9 |
| citric acid in 50% Alkermes modified | Day 21 | 42.8 | 5.1 |
| polymer process 85/15 PLGH (InV | | | |
| 0.25)/50% NMP | | | |
| Group II: 15% Octreotide acetate + | Day 1 | 19.3 | 5.4 |
| citric acid | Day 21 | 60.2 | 5.5 |
| in 50% 85/15 PLGH (InV 0.25) | | | |
| Alkermes + | | | |
| 1.4% CH2C12/50% NMP | | | |
| Group III: 15% Octreotide acetate + | Day 1 | 14.8 | 3.1 |
| citric acid in 50% 85/15 PLGH (InV | Day 21 | 54.9 | 3.5 |
| 0.28) Alkermes/50% NMP | | | |
| Group IV: 15% Octreotide acetate + | Day 1 | 6.5 | 1.1 |
| citric acid in 50% 85/15 PLGH (InV | Day 21 | 42.1 | 10.0 |
| 0.27) APT/50% NMP | | | |
| Group V: 15% Octreotide acetate + | Day 1 | 19.2 | 5.4 |
| citric acid in 50% 85/15 PLGH (InV | Day 21 | 53.6 | 5.7 |
| 0.25) Alkermes/50% NMP | | | |

### Conclusions

This study compared the release of octreotide from ATRIGEL® formulations containing 85/15 PLGH polymers obtained from different suppliers. Also, 85/15 PLGH polymers with different molecular weights, added solvent, and modified preparation technique were evaluated for release characteristics. The implant retrieval data showed that the polymer from Absorbable Polymer Technologies, Inc. (Group IV, InV 0.27) gave the lowest initial drug burst on Day 1 as well as the lowest cumulative release of octreotide by Day 21. A similar molecular weight polymer obtained from Alkermes (Group III, Inv 0.28) gave a higher initial burst and cumulative 21 day release indicating a difference between the two polymers other than molecular weight. However, molecular weight or inherent viscosity did make a difference between 85/15 polymers from the same supplier. An Alkermes polymer with an inherent viscosity of 0.25 gave a higher initial burst than the same polymer with an inherent viscosity of 0.28, even though there was no difference in the cumulative release of the polymers at Day 21. Thus, polymer molecular weight is a significant factor in controlling the initial drug burst. Another important factor in controlling the initial drug burst and cumulative release is the polymer preparation technique. An Alkermes polymer made with a modified preparation technique (Group I) gave a much lower burst (10.5% vs 19.2%) than the same inherent viscosity polymer prepared by the standard procedure (Group V). The addition of a small amount of another solvent (methylene chloride) to one of the polymer formulations gave no major change in initial drug burst.

The tissue irritation effects of the various formulations as determined by macroscopic evaluation of the test sites were none to minimal. On Day 1, minimal erythema and edema was observed in two animals in Group II. On Day 21, minimal vasodilation was observed in two animals in Group II. On Day 21, minimal vasodilation was observed in one animal in Group IV and three animals in Group V. No other tissue macroscopic observations were recorded.

No plasma samples were analyzed for octreotide concentration as it was decided that the implant retrieval data gave sufficient information about the release characteristics of the various formulations.

In summary, the ATRIGEL® formulations with 15% w/w of the octreotide acetate/citric acid mixture appear to give acceptable tissue reactions and drug release characteristics. It also appears that the polymer molecular weight or inherent viscosity is a critical factor in controlling the initial drug burst. However, the polymer preparation process and the polymer supplier appear to be an even more significant factor in controlling both the initial burst and the cumulative release of drug from the ATRIGEL® formulations.

### Example 6

### Randomized, Single-Dose, Open-Label, Single Center, 10-Week Comparative Study of The Pharmacokinetics, Pharmacodynamics and Safety of ATRIGEL®/Octreotide 1-Month Depot (20 mg) and Sandostatin LAR® 1-Month Depot (20 mg) in Healthy Male Subjects

### Introductory Summary

The purpose and primary objective of this study was to compare the pharmacokinetics of ATRIGEL®/Octreotide 1-month depot (20 mg) with Sandostatin LAR® Depot (20 mg) as assessed by plasma concentrations of octreotide. A secondary objective was to compare the pharmacodynamics of ATRIGEL®Octreotide 1-month depot (20 mg) with Sandostatin LAR® Depot (20 mg) as assessed by plasma concentrations of IGF-1. A final objective was to assess the safety of ATRIGEL®/Octreotide 1-month depot (20 mg) and Sandostatin LAR® Depot (20 mg) as determined by plasma levels of thyroid-stimulating hormone (TSH), total and free thyroxine (T₄), fasting glucose and insulin and glucagon; gallbladder ultrasounds; ECGs; clinical lab results and monitoring adverse events.

### Study Design and Methodology Summary

A randomized, single-dose, open-label, single-center, 10-week comparative study of the PK/PD, endocrine profiles and safety of ATRIGEL®/Octreotide 1-month depot (20 mg) and Sandostatin LAR® Depot (20 mg) in healthy male subjects was performed. Twenty (20) healthy male subjects received a single dose of ATRIGEL®/Octreotide 1-month depot (20 mg) or a single dose of Sandostatin LAR® Depot (20 mg) on Day 1. The healthy male subjects were between 18 and 40 years of age (inclusive). Each had a normal medical history and pre-study clinical laboratory measurements either of normal or of no clinical significance. The 1-Month Subcutaneous Depot ATRIGEL®/Octreotide 20 mg, was injected under the skin on the abdomen. Pharmacokinetic, pharmacodynamic, safety and tolerability evaluations were performed, as detailed below.

### Safety and Tolerability

Safety was assessed by spontaneously reported adverse events, physical examinations, vital signs, ECGs and routine clinical laboratory tests (haematology, biochemistry, urinalysis), injection site assessment, gallbladder ultrasound and measurement of TSH, total and free T₄, fasting glucose and insulin and glucagon. Serum TSH, total and free T₄, fasting glucose and insulin and glucagon were summarized from data collected at Day 1 (pre-dose) and Day 24 post-dose and on Days 3, 7, 14, 21, 28 and 70 post-dose to compare results between the two treatments.

### Pharmacokinetic Methods

Twenty-three (23) samples were collected from each subject during the course of the study (70 days) to determine the pharmacokinetics of octreotide in subjects given ATRIGEL®/Octreotide 1-month depot (20 mg) or Sandostatin LAR® Depot (20 mg). Based on the individual octreotide plasma profiles, the following pharmacokinetic parameters were derived using non-compartmental methods: t_{Lag}, Cₘₐₓ, tₘₐₓ, AUC₀₋₂₄ and AUC₀₋ₜ. Values for λ_{z}, t_{1/2} and AUC_{0-inf} were not calculable. Each parameter was summarized by treatment type.

### Pharmacodynamic Methods

Serum IGF-1 levels were summarized for samples collected over 70 days to compare results between the two treatments.

### Statistical Methods

Cₘₐₓ, AUC₀₋₂₄ and AUC₀₋ₜ for octreotide following ATRIGEL®/Octreotide 20 mg (Test) were compared to those following Sandostatin LAR® 20 mg (Reference) using analysis of variance (ANOVA) of log-transforms within the SAS GLM procedure. The statistical model included factors accounting for variation due to treatment. The difference between the mean log-transformed endpoints for each parameter was estimated, together with the 90% confidence interval (CI) for these differences. Similarly serum IGF-1 concentrations at each time point following ATRIGEL®/Octreotide 20 mg (Test) was compared to that following Sandostatin LAR® 20 mg (Reference) using ANOVA of log-transforms within the SAS GLM procedure. The statistical model included factors accounting for variation due to treatment. The difference between the mean log-transformed endpoints for each time point was estimated, together with the 90% CI for these differences.

### Summary Results

### Safety and Tolerability Results:

There were no deaths or serious Adverse Events (AEs) during the study. There was a total of 101 treatment-emergent AEs reported by 20 subjects, of which 80 were mild and 21 moderate in intensity. Sixty-one AEs (60.4% of total) in 10 subjects were observed following ATRIGEL® 20 mg and 40 AEs (39.6% of total) in 9 subjects following Sandostatin LAR® 20 mg. Subject 19 did not report any AEs throughout the study. Fifty-two AEs following ATRIGEL®/Octreotide 20 mg and 28 AEs following Sandostatin LAR® 20 mg were considered to be “certainly, probably/likely or possibly” related to treatment.

The most common AEs were related to the injection site with 23 cases of erythema in 16 subjects at the injection site, which ranged from mild to moderate in intensity. Fifteen cases in 10 subjects followed ATRIGEL®/Octreotide 20 mg, compared to 8 events in 6 subjects following Sandostatin LAR® 20 mg. Sixty-six percent of erythema cases (10 AEs in 10 subjects) following ATRIGEL® formulation administration were reported as moderate in intensity, compared to only one following Sandostatin® LAR product administration. Ten subjects reported palpable masses at the s.c. injection site, mostly following ATRIGEL®/Octreotide 20 mg (8 AEs in 8 subjects). AEs related to local tolerability were not associated with any systemic upset. The higher frequency of local tolerability AEs following s.c. injection of ATRIGEL®/Octreotide 20 mg under the skin of the abdomen compared to i.m. injection of Sandostatin LAR® 20 mg deep into the buttock may have been related to the mode and location of administration.

Other commonly reported AEs were related to gastrointestinal effects (diarrhea, abdominal discomfort, abdominal distension, flatulence and loose stools). These were as predicted from previous studies with octreotide and were similar for both treatments, (12 AEs in 6 subjects) and (10 AEs in 7 subjects), following ATRIGEL®/Octreotide 20 mg and Sandostatin LAR® 20 mg, respectively. Other systemic AEs also appeared to be similar in frequency for both treatments.

There were no clinically significant abnormalities in ECGs, vital signs or during gallbladder ultrasound assessments. Biliary sludge was reported for 40% (4/10) of subjects receiving Atrigel/Octreotide 20 mg and for 50% (5110) of subjects receiving Sandostatin LAR®. However all gallbladders were normal at the post-study examination on Day 70. Fifteen minutes following s.c. injection of ATRIGEL®/Octreotide 20 mg 9 subjects had moderate and 1 subject had severe erythema. By 8 hours post-injection 9 subjects were without and 1 subject had mild erythema. By 35 days all subjects were completely free of erythema symptoms. Fifteen minutes following i.m. injection of Sandostatin LAR® 20 mg 2 subjects had mild, and the other 8 subjects were absent of, erythema. By 8 hours post-injection 9 subjects were without and 1 subject had mild erythema. By 7 days all subjects were absent of erythema with 1 exception of mild erythema on day 42.

Thyroid-stimulating hormone (TSH) was below the normal range for 80% of subjects prior to or on Day 3 following ATRIGEL®/Octreotide 20 mg compared to only 20% of subjects following Sandostatin LAR® 20 mg. Total T₄ concentrations were below the normal range for 30% of subjects who received ATRIGEL®/Octreotide 20 mg on Day 3 compared to 0% of subjects following Sandostatin LAR® 20 mg. For 4 subjects individual glucagon concentrations declined below the normal range prior to or on Day 7, 3 of whom received ATRIGEL®/Octreotide 20 mg. On 51 occasions in 15 subjects insulin decreased below the normal range, 57% (29/51) occurring in subjects who received ATRIGEL®/Octreotide 20 mg and 43% (22/51) in subjects who received Sandostatin LAR® 20 mg. On 18 occasions (out of 29) following ATRIGEL®/Octreotide 20 mg and on 10 occasions (out of 22) after treatment of Sandostatin LAR® 20 mg insulin decreased below the normal range on or before Day 7. The greater number of earlier events following ATRIGEL®/Octreotide 20 mg may have been related to higher initial concentrations of octreotide. All concentrations out of range returned to normal soon afterwards before the post-study assessment and none of the deviations outside the normal range were considered to be clinically significant.

### Pharmacokinetic Results

Mean peak plasma octreotide concentrations of 38.4 and 1.9 ng/mL were achieved at median times of 3.0 and 312.0 hours after dosing of ATRIGEL®/Octreotide 20 mg and Sandostatin LAR® 20 mg, respectively. The difference in magnitude and time of Cₘₐₓ following ATRIGEL®/Octreotide 20 mg was due to the initial burst/pulse delivery of octreotide immediately following injection. Mean AUC₀₋₂₄ was 353.2±142.4 ng.h/mL and 1.8±4.1 ng.h/mL for ATRIGEL®/Octreotide 20 mg and Sandostatin LAR® 20 mg, respectively. Mean values of AUC₀₋ₜ for ATRIGEL®/Octreotide 20 mg were found to be over 5 times greater than for Sandostatin LAR® 20 mg, indicating the improved degree of exposure to octreotide with the novel product. Exposure to octreotide following ATRIGEL®/Octreotide 20 mg was found to be statistically significantly greater compared to Sandostatin LAR®20 mg based on comparisons of Cₘₐₓ and AUC₀₋ₜ.

TABLE 6-1
Summary Pharmacokinetic Parameters Following Administration of
Single s.c. Dose of ATRIGEL ®/Octreotide 20 mg and Single i.m.
Dose of Sandostatin LAR ® 20 mg to Separate Groups of Subjects.

| | | ATRIGEL ® 20 mg | Sandostatin |
|---|---|---|---|
| | Summary | s.c. | LAR ® 20 mg |
| Parameter | Statistic | Depot (n = 10) | i.m. Depot (n = 10) |
| t_{lag} (h) | Median | 0.000 | 0.500 |
| | Range | (0.00-0.00) | (0.00-144.00) |
| Cₘₐₓ (ng/mL) | Mean | 38.370 | 1.884 |
| | SD | 21.408 | 4.119 |
| tₘₐₓ (h) | Median | 3.000 | 312.00 |
| | Range | (2.00-5.00) | (6.00-648.17) |
| AUC₀₋₂₄ | Mean | 353.2 | 1.8 |
| (ng · h/mL) | SD | 142.4 | 4.1 |
| AUC₀₋ₜ | Mean | 1351.6 | 253.7 |
| (ng · h/mL) | SD | 953.3 | 104.0 |

### Pharmacodynamic Results

Mean IGF-1 concentrations declined from ≈95 ng/mL at pre-dose to ≈50 mg/mL (≈50% change from baseline) on Day 7, before returning close to pre-dose levels on Day 49 following ATRIGEL®/Octreotide 20 mg dosing on Day 1. Mean IGF-1 concentrations following dosing of Sandostatin LAR® 20 mg declined from ≈80 ng/mL at pre-dose to a minimum level of ≈50 ng/mL (≈38% change from baseline) on Day 14 before returning close to pre-dose levels on Day 56. IGF-1 levels were statistically significantly greater following administration of ATRIGEL®/Octreotide 20 mg compared to Sandostatin LAR® 20 mg at nominal times of, 4 hours and on Days 21, 49, 56 and 70. At 24 hours and on Day 7 following administration of ATRIGEL®/Octreotide 20 mg IGF-1 levels were statistically significantly lower following treatment of ATRIGEL®/Octreotide 20 mg compared to IGF-1 levels following Sandostatin LAR® 20 mg. All other statistical comparisons were inconclusive based on the conventional confidence interval approach. These results suggest that the sample size was not large enough to allow definitive conclusions to be drawn.

### Summary Conclusions

In general the treatments were well tolerated. There were no deaths or serious adverse events during the study. There was a total of 101 treatment-emergent AEs, 80 of which were mild in intensity. Sixty-one AEs in 10 subjects were observed following ATRIGEL®/Octreotide 20 mg and 40 AEs in 9 subjects following Sandostatin LAR® 20 mg. Most common reported AEs were injection site erythema, injection site nodule and diarrhea.

Fifty-two AEs were “certainly, possibly or probably related” to ATRIGEL®/Octreotide 20 mg and 28 AEs were “certainly, possibly or probably related” to Sandostatin LAR® 20 mg.

Injection site erythema was the most common reported AEs with 23 treatment related occurrences in 16 subjects, 15 of these events in 10 subjects occurred following ATRIGEL®. Injection site nodule (12 AEs in total) (9 in 8 subjects following ATRIGEL®) and diarrhea (12 AEs in total) were also commonly reported AEs following treatment. Following ATRIGEL®/Octreotide 20 mg subjects were completely free of erythema from 35 days post-dose. Throughout the study there were always more than 7 subjects absent of erythema symptoms following Sandostatin LAR® 20 mg.

There were no clinically significant abnormalities in ECG or vital signs. There were no clinically significant abnormalities identified during gallbladder ultrasound assessments. Biliary sludge was reported for a number of subjects, however all gallbladders were normal at the post-study examination on Day 70.

TSH was below the normal range for 80% of subjects prior to or on Day 3 following ATRIGEL®/Octreotide 20 mg compared to only 20% of subjects following Sandostatin LAR® 20 mg. Total T₄ concentrations were below the normal range for 30% of subjects who received ATRIGEL®/Octreotide 20 mg on Day 3 compared to 0% of subjects following Sandostatin LAR® 20 mg. For 4 subjects individual glucagon concentrations declined below the normal range prior to or on Day 7, 3 of whom received ATRIGEL®/Octreotide 20 mg.

On 51 occasions in 15 subjects insulin decreased below the normal range, 57% (29/51) occurring in subjects who received ATRIGEL®/Octreotide 20 mg and 43% (22/51) in subjects who received Sandostatin LAR® 20 mg. On 18 occasions (out of 29) following ATRIGEL®/Octreotide 20 mg and on 10 occasions (out of 22) after treatment of Sandostatin LAR® 20 mg insulin decreased below the normal range on or before Day 7. The greater number of earlier events following ATRIGEL®/Octreotide 20 mg may have been related to higher initial concentrations of octreotide.

All concentrations out of range returned to normal soon afterwards before the post-study assessment and none of the deviations outside the normal range were considered to be clinically significant.

High (38.4 ng/mL) and early (3.0 h) values of Cₘₐₓ following ATRIGEL®/Octreotide 20 mg were due to the initial burst/pulse delivery of octreotide at administration. The effect of this was shown in the comparison of values of AUC₀₋₂₄ which was nearly 200 fold higher for ATRIGEL®/Octreotide 20 mg.

Mean values of AUC₀₋ₜ for ATRIGEL®/Octreotide 20 mg were almost 5 fold higher than for Sandostatin LAR® 20 mg, indicating the improved degree of overall exposure to octreotide with the novel product. Exposure to octreotide following ATRIGEL®/Octreotide 20 mg was found to be statistcically significantly greater compared to Sandostatin LAR® 20 mg based on comparisons of Cₘₐₓ and AUC₀₋ₜ and tₘₐₓ was also found to occur statistically significantly earlier for ATRIGEL®/Octreotide 20 mg.

Mean IGF-1 concentrations following dosing of ATRIGEL®/Octreotide 20 mg declined by a greater magnitude from baseline (approximately 12% greater) and more rapidly (7 Days) compared to IGF-1 concentrations following Sandostatin LAR® 20 mg. The greater effect of ATRIGEL® on IGF-1 may be due to the initial burst of octreotide. However, at most timepoints statistical comparisons of IGF-1 levels were inconclusive suggesting the sample size was not large enough to allow definitive conclusions regarding the level of IGF-1 observed with the two treatments.

### List of Abbreviations and Definition of Terms

ABT Alcohol breath test
AE Adverse event
ALP Alkaline phosphatase
ALT Alanine transferase
AST Aspartate transferase
AUC Area under the plasma concentration-time profile
AUC₀₋₂₄ Area under the plasma concentration-time profile from time 0 to 24 hours post-dose
AUC_{0-inf} Area under the plasma concentration-time profile from time 0 to infinity
CₘₐₓMaximum plasma concentration
CRF Case Report Form
CV(%) Coefficient of variation, expressed as a percentage
DOA Drugs of abuse
ECG Electrocardiogram
FDA Food and Drug Administration
Gamma GT Gamma-glutamyl transferase
GCP Good Clinical Practice
GH Growth Hormone
GLP Good Laboratory Practice
GnRH Gonadotropin-releasing Hormone
GP General practitioner
Hb Haemoglobin
HBsAg Hepatitis B surface antigen
HR Heart rate
ICH International Conference on Harmonisation
IEC Independent Ethics Committee
IGF-1 Insulin-Like Growth Factor-1
i.m. Intramuscular
i.v. Intravenous
Ke Apparent terminal phase rate constant
LDH Lactate dehydrogenase
LH Luteinizing Hormone
Max Maximum
MCH Mean cell haemoglobin
MCHC Mean cell haemoglobin concentration
MCV Mean cell volume
MedDRA Medical Dictionary for Regulatory Activities
mg Milligram
Min Minimum
Min Minute
mL Millilitre
N Number of observations
NMP N-methyl-2-pyrrolidone
PD Pharmacodynamic
PLGH Poly(DL-lactide-co-glycolide) with a Carboxylic Acid End Group
PK Pharmacokinetic
QA Quality Assurance
QC Quality Control
SAE Serious adverse event
s.c. Subcutaneous
SD Standard deviation
SE Standard error
SOP Standard Operating Procedure
T₄ Thyroxine
TA Test Article
TSH Thyroid Stimulating Hormone
t_{lag} Time before start of absorption (timepoint immediately prior to time of first quantifiable plasma concentration)
tₘₐₓ Time to reach maximum plasma concentration
U.K. United Kingdom
U.S.A United States of America
USP United States Pharmacopoeia
VICF Volunteer Informed Consent Form
VIP Vasoactive Intestinal Peptide

### 1. Study Introduction

### 1.1 Objectives

ATRIGEL®/Octreotide is a product being developed for the long-term treatment of diarrhea and flushing episodes associated with metastatic carcinoid tumors (Carcinoid Syndrome). Currently, patients with metastatic carcinoid syndrome who are treated with octreotide receive a Sandostatin LAR® Depot intramuscular (i.m.) injection along with daily subcutaneous (s.c.) injections of Sandostatin to reach therapeutic levels. The release profile of the ATRIGEL®/Octreotide product differs from the innovator product as a “burst” of octreotide is released upon injection in preclinical studies. This study compares the time to onset of therapeutic doses of octreotide with 2 different 20 mg extended release delivery formulations, ATRIGEL®/Octreotide and Sandostatin LAR® Depot.

### 1.2 Clinical Use of Octreotide

Octreotide has been available in the United States and Europe for a number of years and is indicated to treat the symptoms associated with metastatic carcinoid tumors (i.e., flushing and diarrhea), and the symptoms associated with vasoactive intestinal peptide (VIP) secreting adenomas (i.e., watery diarrhea). In addition, octreotide substantially reduces GH and IGF-1 (somatomedin C) levels in patients with acromegaly. Octreotide is currently available as Sandostatin® Injection, a daily s.c. injection, or as Sandostatin LAR® Depot, a monthly, depot i.m. injection.

Octreotide is the acetate salt of a cyclic octapeptide. It is a long-acting peptide with pharmacologic actions similar to those of the natural hormone, somatostatin. It is an even more potent inhibitor of growth hormone (GH), glucagon, and insulin release than somatostatin. Like somatostatin, it also suppresses luteinizing hormone (LH) response to gonadotropin-releasing hormone (GnRH), decreases splanchnic blood flow, and inhibits release of serotonin, gastrin, VIP, secretin, motilin, and pancreatic polypeptide. Octreotide is generally well tolerated. The most commonly found adverse effects are abdominal discomfort, loose stools or diarrhea, mild malabsorption, flatulence, and nausea. The development of cholesterol gallstones with prolonged octreotide treatment is the most serious adverse effect. Octreotide treatment may lower insulin and glucagon levels and could affect glucose homeostasis.

### 1.3 Clinical Use of the ATRIGEL® Delivery System

ATRIGEL® Delivery System consists of biodegradable polymers dissolved in biocompatible carriers. The formulation is based on the desired time frame of sustained release and on the nature of the drug being delivered. The ATRIGEL® Delivery System is currently used in the FDA approved products ELIGARD™ (one, three and four-month subcutaneous depot formulations of leuprolide acetate) and ATRIDOX® (doxycycline hyclate applied to the periodontal pocket). Clinical studies and post-marketing experience with these products demonstrate that the ATRIGEL® Delivery System itself is well tolerated and provides consistent, sustained release of the incorporated drug over the designated dosing period.

1.4 Non-Clinical Results with ATRIGEL®/Octreotide

The potential toxicity, toxicokinetics, local irritation and octreotide release kinetics as well as pharmacodynamics of ATRIGEL®/Octreotide have been investigated in pre-clinical animal studies. Five GLP and 11 non-GLP non-clinical studies with ATRIGEL®/Octreotide in rats, rabbits, dogs and monkeys have been completed. The results of these studies verify safety with only slight erythema and edema being observed at the injection sites. In addition, the expected octreotide release and endocrine response were observed (plasma octreotide levels were above the therapeutic level and serum IGF-1 levels were suppressed throughout the study).

Taken together, the pre-clinical and clinical safety experiences with the ATRIGEL® Delivery System and the drug substance octreotide suggest that the ATRIGEL®/Octreotide product should have a favourable safety profile. This study compares the safety, pharmacokinetic and pharmacodynamic/endocrine profiles of octreotide after administration of ATRIGEL®/Octreotide 1-month depot (20 mg) or Sandostatin LAR® Depot (20 mg) to healthy male subjects.

### 2. Study Objectives

1. To compare the PK of ATRIGEL®/Octreotide 1-month depot (20 mg) to Sandostatin LAR® Depot (20 mg) as assessed by plasma concentrations of octreotide.
2. To compare the PD of ATRIGEL®/Octreotide 1-month depot (20 mg) to Sandostatin LAR® Depot (20 mg) as assessed by plasma concentrations of IGF-1.
3. To assess the safety of ATRIGEL®/Octreotide 1-month depot (20 mg) and Sandostatin LAR® Depot (20 mg) as determined by plasma levels of thyroid-stimulating hormone (TSH), total and free thyroxine (T₄), fasting glucose and insulin and glucagon; gallbladder ultrasounds; ECGs; clinical lab results and monitoring adverse events.

### 3. Investigation Plan

### 3.1 Overall Study Design

This was a randomized, single-dose, open-label, single-center, 10-week comparative study of the PK/PD, endocrine profiles and safety of ATRIGEL®/Octreotide 1-month depot (20 mg) and Sandostatin LAR® Depot (20 mg) in healthy male subjects. Twenty (20) healthy male subjects received a single dose of ATRIGEL®/Octreotide 1-month depot (20 mg) or a single dose of Sandostatin LAR® Depot (20 mg) on Day 1. Each subject participated in one treatment group only.

After subjects had given their informed consent, they attended the Clinical Unit for a screening visit within 4 weeks prior to study drug administration. Subjects were required to abstain from alcohol and smoking 24 hours prior to screening.

Screening consisted of an interview and the following assessments: a) Demographics (sex, date of birth, age, height, frame size, elbow width, race); b) Medical history; c) Social History (current alcohol intake, caffeine intake and smoking status); d) Check of prior and concomitant medications; e) An electrocardiogram (ECG); f) A physical examination: height, weight, blood pressure (BP), heart rate, respiratory rate and temperature; and g) Clinical laboratory tests: hematology, clinical chemistry, urinalysis, virology (hepatitis B surface antigen, human immunodeficiency virus (HIV) antibodies, Hepatitis C antibodies), urine drugs of abuse screen (DOA) and an alcohol breath test (ABT).

Following successful completion of screening, the subjects were randomized and enrolled onto the study. The main part of the study consisted of 2 regimens of identical design, differing only in the allocated treatment.

Day 0: The subjects were admitted to the Clinical Unit the day before dosing having fasted for 6 hours prior to visit with the exception of water. The following procedures were performed on admission: 1) Clinical Laboratory Tests (hematology, biochemistry and urinalysis); 2) DOA; 3) ECG; and 4) Gallbladder Ultrasound.

Volunteers were assessed to ensure that they still fulfilled the inclusion and exclusion criteria for the study and successful volunteers were randomized to receive a single dose of either ATRIGEL®/Octreotide (1-month depot, 20 mg) or Sandostatin LARD (1-month depot, 20 mg). Subjects were required to fast from 2300 hours on Day −1 until the last blood sample was taken on the morning of Day 2. Water was not restricted and glucose drinks were provided.

Day 1: On Day 1, prior to dosing subjects had their vital signs measured (temperature, supine BP, HR and respiratory rate). Blood samples were taken pre-dose to quantify octreotide, IGF-1, TSH and total and free T₄. Blood samples were also taken for fasting glucose and insulin and glucagon. An assessment of the injection site was also completed.

Subjects were dosed with a single dose of either ATRIGEL®/Octreotide (1-month depot, 20 mg) or Sandostatin LAR® Depot (1-month depot, 20 mg) according to the randomization schedule. Following dosing, subjects remained at the unit for approximately 24 hours for collection of blood samples for PK/PD and to monitor safety by clinical observation, ECGs, measurement of vital signs and collection of adverse events. An assessment of the injection site was done at 15 and 30 minutes post-dose, hourly until 4 hours post-dose and at 8, 12, 16 and 24 hours post-dose. Blood samples for octreotide and TSH, total and free T₄, fasting glucose and insulin and glucagon analysis on Day 1 were taken hourly for the first 8 hours post-dose and every 4 hours thereafter until 24 hours post-dose. IGF-1 samples were taken every 4 hours post-dose until 24 hours post-dose (i.e., 4, 8, 12, 16, 20 and 24 h post-dose).

Days 3, 7, 14, 21, 28, 35, 42, 49, 56 and 70: Subjects were instructed to abstain from alcohol and smoking for 24 hours prior to morning visits on Days 3, 7, 14, 21, 28, 35, 42, 49, 56 and 70 and having fasted for 2 hours (with the exception of water) prior to morning visits on Days 3, 7, 14, 21 and for 6 hours prior to the morning visits on Days 28 and 70. Blood samples for octreotide and IGF-1 analysis were collected on Days 3, 7, 14, 21, 28, 35, 42, 49, 56 and 70. Analysis of TSH, total and free T₄, fasting glucose and insulin and glucagon was done for samples collected on Days 3, 7, 14, 21, 28 and 70. Safety was monitored by clinical observation, evaluation of the injection site, measurement of vital signs and collection of adverse events. In addition, on Days 28 and 70 subjects underwent ECG analysis, gallbladder ultrasound and blood was taken to conduct clinical chemistry and hematology panels. Urine was also collected for urinalysis. Random alcohol breath tests were done at each morning visit and concomitant medications were reviewed. The final safety assessment or post-study medical was done on the morning visit on Day 70.

3.2 Discussion of Study Design: The design of the study was deemed appropriate to achieve the objectives set out for the study.

### 3.3 Selection of Study Population

Healthy subjects were recruited from the Medeval Limited volunteer panel. Twenty male volunteers were enrolled in this study. Study subjects were admitted into the study at the discretion of the Investigator based upon medical history and findings of the screening interview and examination. Each subject was expected to participate in the entire duration of the study. Each subject was entered into the study based upon the following criteria:

### 3.3.1 Inclusion Criteria

1. Subject had read and signed the informed consent agreement.
2. Subject was a healthy male, 18-40 years of age.
3. Subject was able to follow verbal and/or written instructions, and return to the center for specified study visits.
4. Subject was free from any clinically significant abnormality (i.e., clinical results fell within the Medeval normal ranges or not considered clinically significant by the Medical Director) on the basis of medical history, physical examination (including height and weight and vital signs) and laboratory evaluations.

### 3.4 Treatments

### 3.4.1 Treatments Administered

Subjects received either a single dose of s.c. ATRIGEL®/Octreotide (1-20 month depot, 20 mg) or i.m. Sandostatin LAR® Depot (1-month depot, 20 mg) as determined by a previously generated randomization schedule which split subjects on a 1:1 basis between treatments.

### 3.4.2 Identity of Investigational Products

ATRIGEL®/Octreotide (AL3928.01) was supplied in two separate, sterile syringes and was mixed immediately prior to administration. One syringe contained the polymer formulation, and the other contained the octreotide peptide. The syringes were joined via the Luer-Lok® connections on the syringes, and the formulation was passed between syringes until a homogenous mixture was obtained. Due to the losses during mixing and administration, an overage of polymer solution and drug substance was provided to ensure delivery of 20 mg of octreotide in approximately 0.2 mL of formulation.

Sandostatin LAR® Depot (20 mg) was supplied in two separate, sterile vials which were mixed immediately prior to administration. One vial contained octreotide uniformly distributed within microspheres of the biodegradable glucose star polymer, D,L-lactic and glycolic acids copolymer, and the other vial contained the sterile diluent (water for injection).

TABLE 6-2
Study Drug Batch Numbers and Expiry Dates

| Product | Batch Number | Expiry Date |
|---|---|---|
| ATRIGEL ®/Octreotide | 1598 (Final Product) | 27/11/2003 |
| Sandostatin LAR ® Depot (20 mg) | 2 × 016G7566, | July 2005 |
| | 9 × 017G7574 | (Retest Date) |

The test articles were stored in a refrigerator at 2-8° C. (normal refiigerator temperature) in a secure, controlled-access area until used. All supplies were maintained under adequate security by the Pharmacy Technician, who kept a cumulative inventory and dispensing records.

### 3.4.3 Method of Assigning Subjects to Treatment Groups

The randomisation number was only allocated after each subject successfully completed screening and was found eligible for entry onto the study. From the screening visit until the allocation of a randomization number, subjects were identified by their initials and date of birth.

### 3.4.4 Selection of Doses in the Study

ATRIGEL®/Octreotide (20 mg) was selected for dosage to healthy volunteers as a standard comparison to the currently administered drug in patients, Sandostatin LARD Depot (20 mg).

### 3.4.5 Selection and Timing of Dose for Each Subject

Subjects received either s.c. ATRIGEL®/Octreotide or i.m. Sandostatin LAR® Depot (20 mg) according to a previously generated randomization schedule. Subjects were administered the study drug between 8:00 and 10:00 hours on Day 1.

3.4.6 Blinding: This study was conducted in an open label manner.

### 3.5 Clinical Laboratory Analyses

Serum TSH, Total And Free T₄, Fasting Glucose, Insulin and Glucogon: Samples for serum TSH, total and free T4, fasting glucose and insulin and glucagon were collected on Day 1 (pre-dose) and 24 hours post-dose and on Days 3, 7, 14, 21, 28 and 70. Results were summarized using descriptive statistics.
Hematology: Two mL whole blood samples were taken at screening, Day −1, 24 hours post-dose and on Days 28 and 70 and transferred to 5 mL EDTA tubes in order for the following tests to be carried out:

| | Differential Count |
|---|---|
| Hematology (whole blood sample): | (whole blood sample): |
| Hemoglobin (Hb) | Basophils |
| Mean corpuscular haemoglobin (MCH) | Eosinophils |
| MCH concentration (MCHC) | Lymphocytes |
| Mean corpuscular volume | Monocytes |
| Packed cell volume | Neutrophils |
| Platelet count | |
| Red cell count | |
| White cell count | |

Biochemistry: Seven mL or 10 mL (at screening) whole blood samples were taken at screening, Day −1, 24 hours post-dose and on Days 28 and 70 and transferred into Z10 tubes in order for the following tests to be carried out:

Biochemistry (serum sample):

Alanine amino transferase (ALT)
Albumin
Alkaline phosphatases
Aspartate amino transferase (AST)
Bicarbonate
Total bilirubin
Calcium
Cholesterol
Chloride
Creatinine
Globulin
Glucose
Gamma-glutamyl transferase (Gamma GT)
Inorganic Phosphorous
Potassium
Sodium
Total proteins
Total bilirubin
Urea
Uric acid

Urinalysis: Twenty-five mL or 2×25 mL (at screening) urine samples were taken at screening Day −1, 24 hours post-dose and on Days 28 and 70 in order for the following tests to be carried out:

| Urinalysis: | Microscopy¹: |
|---|---|
| Blood (free Hb) | Casts |
| Glucose | Epithelial cells |
| Ketones | Red blood cells |
| Bilirubin | White blood cells |
| Nitrite | |
| pH | |
| Protein | |
| Urobilinogen | |
| Specific Gravity | |

¹Microscopy only performed in the event of abnormal urinalysis

Virology: The following tests were performed at screening only: Virology (serum sample): Hepatitis B surface antigen, Hepatitis C antibodies, HIV 1 and HIV 2 antibodies.
Drug Screen: All subjects provided a sample of expiratory air, which was tested for alcohol abuse using an ABT at screening and random subjects provided samples on Day −1, Days 3, 7, 14, 21, 28, 35, 42, 49 and 56.

Urine drugs of abuse testing was carried out at screening and on Day −1 using the following tests. At screening a urine sample was tested using a SYVA test kit in the Clinical Pathology Unit and on Day-1 a sample was tested using a SYVA RAPID test kit or a Triage kit. The following drugs were tested for:

| SYVA Test: | SYVA RAPID Test: | Triage Kit: |
|---|---|---|
| Amphetamines | Amphetamines | Amphetamines |
| Barbiturates | Barbiturates | Barbiturates |
| Benzodiazepines | Benzodiazepines | Benzodiazepines |
| Cannabinoids | Cannabinoids | Cannabinoids |
| Cocaine | Cocaine | Cocaine |
| Opiates | Opiates | Opiates |
| | Methamphetamines | Methadone |
| | | Tricyclic |
| | | antidepressants |

The following sample volumes were taken for the tests listed above:
a) Hematology: 2 mL blood sample into EDTA tubes;
b) Biochemistry (screening) including virology: 10 mL blood sample into plain tubes;
c) Biochemistry (pre-dose,): 7 mL blood sample into plain tubes;
d) Urinalysis: 20 mL urine samples into universal containers; and
e) Drugs of abuse: 20 mL urine samples into universal containers.

In the event of unexplained abnormal laboratory test values, the tests were repeated and followed up until they returned to normal range or an adequate explanation for the abnormality was found.

Drug Concentration Measurements: Twenty-three samples were collected from each subject during the course of the study to determine the pharmacokinetics of octreotide in subjects given ATRIGEL®/Octreotide 1-month depot (20 mg) or Sandostatin LAR® Depot (20 mg). EDTA tubes were used to collect approximately 3 mL samples on Day 1 (pre-dose, and 1, 2, 3, 4, 5, 6, 7, 8, 12, 16, 20 and 24 hours post-dose) and on Days 3, 7 14, 21, 28, 35, 42, 49, 56 and 70. The actual time of each blood collection was recorded.
Pharmacodynamic Measurements: Samples for serum IGF-1 levels were collected on Day 1 (pre-dose, 4, 8, 12, 16, 20 and 24 hours post-dose) and on Days 3, 7, 14, 21, 28, 35, 42, 49, 56 and 70. Results were summarized using descriptive statistics.
Pharmacokinetic Data: Pharmacokinetic analysis by standard model-independent methods was performed by a pharmacokineticist in the Department of Pharmacokinetics at Medeval Limited using WinNonlin™ Professional Version 4.0. The following pharmacokinetic parameters were to be determined using the actual blood sampling times following drug administration:
a) the maximum observed plasma concentration (Cₘₐₓ);
b) the corresponding time of the observed Cₘₐₓ (tₘₐₓ);
c) the time before start of absorption (t_{lag});
d) area under the plasma concentration-time curve from time zero to 24 hours post-dose (AUC₀₋₂₄);
e) area under the plasma concentration-time curve from time zero to the last quantifiable time point post-dose (AUC₀₋ₜ);
f) area under the plasma concentration-time curve from time zero to infinity (AUC_{0-inf});
g) the apparent plasma terminal phase rate constant (Kₑ); and
h) elimination half-life (t_{1/2}).

Cₘₐₓ and tₘₐₓ were identified by examination of the plasma profiles for each volunteer and each dosing period: the values were taken as the co-ordinates of the data point with the highest concentration. The t_{lag} was identified as the time point immediately prior to the first quantifiable drug concentration. AUC₀-∞₂₄ and AUC₀₋ₜ were calculated using the linear trapezoidal rule.

Individual plasma concentrations were summarised for each sampling time, for each treatment using N, arithmetic mean (Mean), SD, CV(%), median, Min and Max values. Mean concentrations at any individual time point were only to be calculated if at least ⅔ of the individual values were quantified at this time point. Values that were below the limit of quantification (BLQ) of the assay were set to zero for the calculation of these mean values.

Individual linear and log-linear plasma concentration-time profiles were produced using actual sampling times. Mean (+SD) linear and log-linear plasma concentrations profiles were also produced. In cases where a mean value was not calculable, the value was to be set to missing for plotting purposes. All derived individual pharmacokinetic parameters are listed in the report appendices and summarised for each treatment.

Pharmacodynamic Data: Serum IGF-1 concentrations were measured on Day 1 (pre-dose, 4, 8, 12, 16, 20 and 24 hours post-dose) and on Days 3, 7, 14, 21, 28, 35, 42, 49, 56 and 70. Results were summarized using descriptive statistics and a mean serum IGF-1 concentration-time profile was plotted.
Sample Size: The sample size for this study was not determined by formal statistical methods, but was deemed a reasonable size to address the objectives of the study. Ten subjects in each group was deemed sufficient for the evaluation of the safety, tolerability, pharmacokinetics, and pharmacodynamics of s.c. ATRIGEL®/Octreotide 1-month Depot (20 mg) and i.m. Sandostatin LAR® Depot (20 mg).
Pharmacokinetic Parameters: Individual plasma octreotide concentrations were summarized by descriptive statistics of n, arithmetic mean, SD, CV(%), median, minimum, maximum and 95% confidence intervals (CI). All BLQ values were set to zero for the purpose of calculating descriptive statistics. If at any time-point ⅓ or more of subjects had BLQ values, descriptive statistics were not to be calculated at that time-point.

The pharmacokinetic parameters were listed by subject and treatment and summarized using descriptive statistics of n, arithmetic mean, SD, CV (%), median, minimum, maximum and 95% CI. Only n, median, minimum and maximum were reported for tₘₐₓ.

The pharmacokinetic parameters Cₘₐₓ, AUC₀₋₂₄ and AUC_{0-inf} of octreotide following administration of ATRIGEL®/Octreotide 20 mg (Test) were compared to those of octreotide following administration of Sandostatin LAR® 20 mg (Reference) using analysis of variance (ANOVA) of log-transforms within the SAS GLM procedure. The statistical model included factors accounting for variation due to treatment. The difference between the mean log-transformed endpoints was estimated, together with the 90% CI for these differences. The procedure was carried out using the LSMEANS statement. The results were back-transformed to give point estimates of the geometric mean ratios (Test/Reference) and associated 90% CI for each pharmacokinetic parameter.

The tₘₐₓ was analyzed using the non-parametric Wilcoxon's matched pairs test using the UNIVARIATE procedure in SAS on the differences in tₘₐₓ (Test—Reference) for each subject. Statistical significance was considered at the 5% level.

Pharmacodynamic Parameters: Individual serum IGF-1 concentrations were summarized by descriptive statistics of n, arithmetic mean, SD, CV(%), median, minimum, maximum and 95% CI. BLQ values were set to half the value of the limit of quantitation value for the purpose of calculating descriptive statistics. If at any time-point ⅓ or more of subjects had BLQ values, descriptive statistics were not calculated at that time-point.

The serum IGF-1 concentration at each nominal time point following administration of ATRIGEL®/Octreotide 20 mg (Test) was compared to that of octreotide following administration of Sandostatin LAR® 20 mg (Reference) using ANOVA of log transforms within the SAS GLM procedure. The statistical model included factors accounting for variation due to treatment. The difference between the mean log-transformed endpoints was estimated, together with the 90% CI for these differences. The procedure was carried out using the LSMEANS statement. The results were back-transformed to give point estimates of the geometric mean ratios (Test/Reference) and associated 90% CI for each time point.

### 3.8 Changes in the Conduct of the Study or Planned Analyses

Following dosing of Sandostatin LARD 20 mg, plasma levels of octreotide were BLQ for the majority of the profile. Due to this, embedded BLQ values normally excluded from analysis were instead set to zero and used as part of the pharmacokinetic analysis data set. Due to the nature of the concentration time profiles; λ_{z} and subsequently t_{1/2} and AUC_{0-∞} were unable to be calculated for all subjects following dosing of ATRIGEL®/Octreotide 20 mg and Sandostatin LAR® 20 mg. Plasma octroetide summary statistics following ATRIGEL®/Octreotide 20 mg at 480, 648 and 816 hours were calculated with more than ⅓ of concentrations being BLQ. Similarly following Sandostatin LAR® 20 mg summary concentrations at 1, 2, 3, 4, 6, 49 and 144 hours were calculated with more than ⅓ of concentrations being BLQ. For the mean linear and log-linear concentration-time profile following Sandostatin LAR® 20 mg summary statistics where all concentrations were BLQ, mean values of zero were plotted at time points of 5, 7, 8, 12, 16, 20 and 24 hours post-dose. The statistical comparison of AUC₀₋₂₄ was not able to be done due to there being only 2 values available for subjects who received Sandostatin LAR® 20 mg.

### 4. Study Subjects

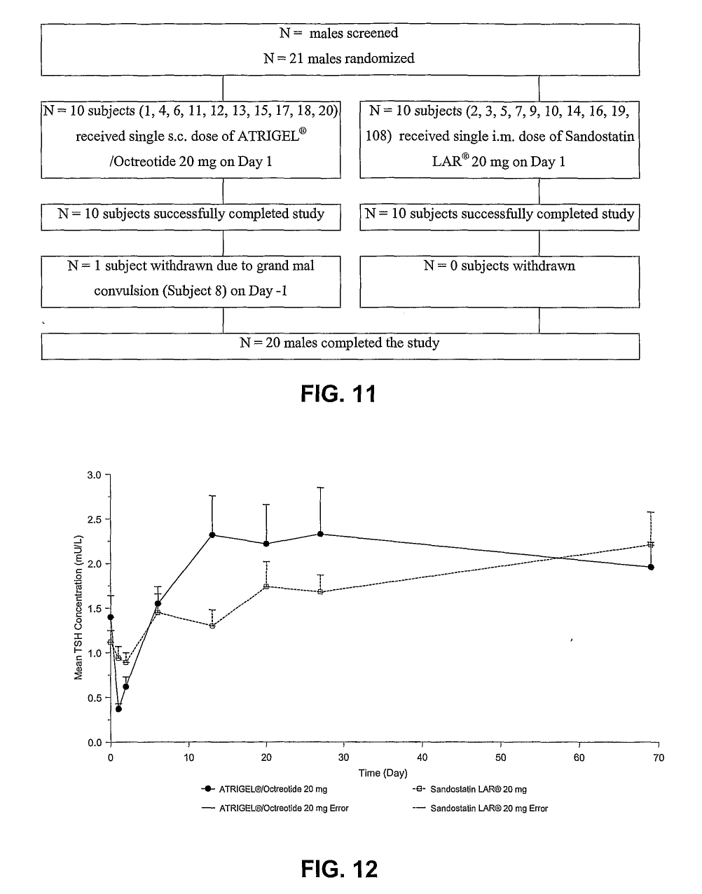

4.1 Disposition of Subjects: The disposition of the subjects who were enrolled in the study is given in FIG. 11.

### 5. Safety Evaluation

Relevant data for subjects have been included in Section 8 and the corresponding tables.

### 5.1 Injection Site Assessments

There appeared to be more incidences of erythema following ATRIGEL®/Octreotide 20 mg compared to Sandostatin LAR® 20 mg. Fifteen minutes following s.c. injection of ATRIGEL®/Octreotide 20 mg 9 subjects had moderate and 1 subject had severe erythema. By 8 hours post-injection 9 subjects were without and 1 subject had mild erythema. By 35 days all subjects were completely free of erythema symptoms.

Fifteen minutes following i.m. injection of Sandostatin LAR® 20 mg 2 subjects had mild and the other 8 subjects were absent of erythema. By 8 hours post-injection 9 subjects were without and 1 subject had mild erythema. By 7 days all subjects were absent of erythema with 1 exception of mild erythema on Day 42. Throughout the study there were always more than 7 subjects absent of erythema symptoms following Sandostatin LAR® 20 mg.

### 5.2 TSH, Total T4 and Free T4

Mean (±SE) linear TSH plasma concentration-time profiles following the two treatments (Administration of Single s.c. Doses of ATRIGEL®/Octreotide 20 mg and Single i.m. Doses of Sandostatin LAR® 20 mg to Separate Groups of Subjects) are given in FIG. 12. Endocrine assessment summary statistics for TSH, total T₄ and free T₄ are given in Section 8, Tables 6-26 to 6-28. Following ATRIGEL®/Octreotide 20 mg, mean TSH concentrations appeared to decrease rapidly from 1.4 mU/L pre-dose to 0.37 mU/L before returning to pre-dose levels on Day 7. There was only a slight decrease in mean TSH concentrations following Sandostatin LAR® 20 mg from 1.1 mU/L to 0.89 mU/L, however mean concentrations after Day 7 were greater than pre-dose levels. Following ATRIGEL®/Octreotide 20 mg, TSH concentrations at Day 2 were slightly below the lower limit of 0.5 mU/L for 80% (8/10) of subjects compared to only 10% (1/10) following Sandostatin LAR® 20 mg. On Day 3, TSH concentrations were less than the lower limit for 20% (2/10) of subjects following ATRIGEL®/Octreotide 20 mg and for 10% (1/10) following treatment of Sandostatin LAR® 20 mg. TSH concentrations were above the upper limit of 5.0 mU/L for Subject 12 (ATRIGEL®/Octreotide 20 mg) on Days 14, 21 and 28.

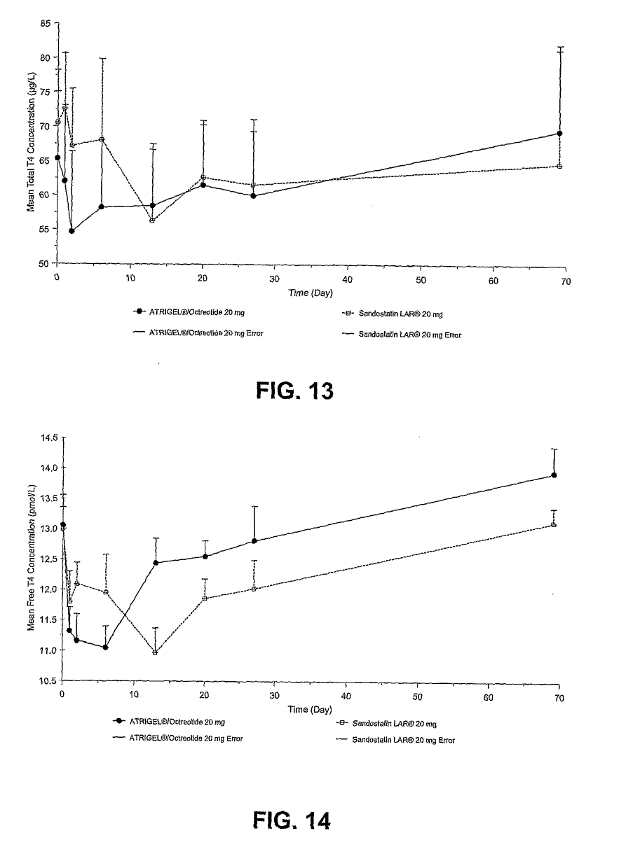

Mean total T₄ concentrations declined slightly from mean pre-dose levels of 65.33 μg/L following ATRIGEL®/Octreotide 20 mg to a minimum value of 54.7 μg/L on Day 3 before subsequently returning close to pre-dose levels by Day 21. Following Sandostatin LAR® 20 mg total T₄ concentrations appeared to decline steadily from 70.6 μg/L pre-dose to 56.3 μg/L on Day 14 post-dose. There was no indication of returning to pre-dose levels by Day 70. Total T₄ concentrations following ATRIGEL®/Octreotide 20 mg were lower than the normal range of 45 μg/L on Day 3 for 30% (3/10) of subjects. After treatment of Sandostatin LAR® 20 mg only total T₄ concentrations for Subject 2 on Day 70 and for Subject 14 on Day 14 were below the normal range. The data in FIG. 13 illustrate mean (+SE) total T₄ concentration-time profiles following administration of single s.c. doses of ATRIGEL®/Octreotide 20 mg and single i.m. doses of Sandostatin LAR® 20 mg to separate groups of subjects.

Mean (+SE) linear free T₄ plasma concentration-time profiles following the two treatments are given in FIG. 14. Mean free T₄ concentrations declined slightly following both treatments before steadily increasing. There were only 2 assessments that revealed individual concentrations to be outside the normal range. Free T₄ was less than 9.1 μmol/L on Day 3 for Subject 1 (ATRIGEL®/Octreotide 20 mg) and on Day 14 for Subject 19 (Sandostatin LAR® 20 mg).

Concentrations of TSH, total T₄ and free T₄ measured outside the normal range were mostly in subjects who received ATRIGEL®/Octreotide 20 mg with the exception of single observations for Subjects 2, 10, 14, 16 and 19 who received Sandostatin LAR® 20 mg. None of the measurements outside the normal range for TSH, total T₄ and free T₄ were clinically significant.

### 5.3 Fasting Glucose and Insulin and Glucagon

Endocrine assessment summary statistics for fasting glucose and insulin and glucagon were taken and evaluated. Mean (+SD) concentrations for fasting glucose and insulin and glucagon are given in Table 6-3 below. Mean values of fasting glucose were similar throughout the study following both ATRIGEL® and Sandostatin LAR® treatments. There were no individual assessments that were above or below the normal range for fasting glucose. Mean pre-dose levels of insulin were 5.12 and 5.10 mIU/L for ATRIGEL® and Sandostatin LAR® treatments, respectively. Mean concentrations appeared to decline following ATRIGEL®/Octreotide 20 mg to a minimum value of 2.96 mIU/L on Day 2, before returning close to pre-dose levels on Day 21. Mean insulin levels decreased to 3.64 mIU/L on Day 2 following Sandostatin LAR® 20 mg and appeared to fluctuate between this value and pre-dose levels until the post-study medical on Day 70. Individual assessments of insulin revealed concentrations to fall below the normal range of 2.5 mIU/L on 51 occasions, 57% (29/51) following ATRIGEL®/Octreotide 20 mg and 43% (22/51) following Sandostatin LAR® 20 mg. On 18 occasions (out of 29) after administration of ATRIGEL®/Octreotide 20 mg and on 10 occasions (out of 22) after treatment of Sandostatin LAR® 20 mg insulin decreased below the normal range on or before Day 7. The greater number of earlier events following ATRIGEL®/Octreotide 20 mg may have been related to higher initial concentrations of octreotide after this treatment. Mean glucagon concentrations reached minimum levels of 64.1 and 74.2 pg/mL on Day 7 following ATRIGEL®/Octreotide 20 mg and Sandostatin LAR® treatments, respectively, before returning close to pre-dose levels of approximately 83 pg/mL after Day 14. For 4 subjects individual glucagon concentrations declined below the normal range of 50 pg/mL prior to or on Day 7; 30% (3/10) of these subjects received ATRIGEL®/Octreotide 20 mg and 10% (1/10) received Sandostatin LAR®20 mg. None of the measurements outside the normal range for fasting glucose and insulin and glucagon were clinically significant.

TABLE 6-3
Mean (+SD) Fasting Glucose, Insulin and Glucagon Concentrations Following Administration of Single s.c.
Doses of ATRIGEL ®/Octreotide 20 mg and Single i.m. Doses of Sandostatin LAR ® 20 mg to
Separate Groups of Subjects

| | Fasting Glucose (mmol/L) | | Fasting Insulin (mIU/L) | | Glucagon (pg/mL) | |
|---|---|---|---|---|---|---|
| | ATRIGEL ®/ | Sandostatin | ATRIGEL ®/ | Sandostatin | ATRIGEL ®/ | Sandostatin |
| Day (Visit) | Octreotide 20 mg | LAR ® 20 mg | Octreotide 20 mg | LAR ® 20 mg | Octreotide 20 mg | LAR ® 20 mg |
| Day 1 | 5.33 ± 0.42 | 5.29 ± 0.31 | 5.12 ± 1.93 | 5.10 ± 1.87 | 82.4 ± 23.6 | 84.6 ± 17.8 |
| (Pre-Dose) | | | | | | |
| Day 2 | 5.25 ± 0.72 | 5.03 ± 0.31 | 2.96 ± 0.89 | 3.64 ± 1.46 | 73.8 ± 16.1 | 84.0 ± 16.4 |
| Day 3 | 5.56 ± 0.40 | 5.56 ± 0.47 | 3.40 ± 1.42 | 5.23 ± 2.95 | 80.2 ± 27.7 | 74.7 ± 21.8 |
| Day 7 | 5.66 ± 0.28 | 5.49 ± 0.44 | 3.11 ± 0.94 | 4.17 ± 2.06 | 64.1 ± 16.1 | 74.2 ± 12.4 |
| Day 14 | 5.65 ± 0.36 | 5.62 ± 0.68 | 3.97 ± 1.65 | 3.93 ± 2.20 | 81.7 ± 16.6 | 88.4 ± 19.0 |
| Day 21 | 5.69 ± 1.04 | 5.77 ± 0.63 | 5.54 ± 5.96 | 4.82 ± 2.06 | 84.4 ± 15.2 | 88.1 ± 12.3 |
| Day 28 | 5.66 ± 0.52 | 5.62 ± 0.41 | 4.50 ± 1.84 | 3.68 ± 1.27 | 83.8 ± 23.1 | 87.3 ± 10.3 |
| Day 70 | 5.22 ± 0.37 | 5.54 ± 0.31 | 4.54 ± 1.68 | 5.23 ± 3.98 | 86.9 ± 21.0 | 85.1 ± 18.2 |

### 5.4 Clinical Laboratory Evaluations

In total there were 189 laboratory values that were outside the normal range, 155 of which were classified as being of no clinical significance. Thirty-three abnormal results were classified as being potentially clinically significant, due to unknown cause, 17 for the ATRIGEL® treatment group and 16 for the Sandostatin LAR® treatment group.

### 5.5 Safety Conclusions

1) In general the treatments were well tolerated. There were no deaths or serious adverse events during the study.
2) There were a total of 101 treatment-emergent AEs, 80 of which were mild and 21 moderate in intensity. Sixty-one AEs in 10 subjects were observed following ATRIGEL®/Octreotide 20 mg and 40 AEs in 10 subjects following Sandostatin LAR® 20 mg. Most common reported AEs were injection site erythema, injection site nodule and diarrhea.
3) Fifty-two AEs were “certainly, possibly or probably related” to ATRIGEL®/Octreotide 20 mg; 28 AEs were “certainly, possibly or probably related” to Sandostatin LAR® 20 mg.
4) Injection site erythema was the most common reported AEs with 23 treatment related occurrences in 16 subjects, 15 of these events in 10 subjects occurred following ATRIGEL®/Octreotide 20 mg. Injection site nodule (12 AEs in total) (9 in 8 subjects following ATRIGEL®/Octreotide 20 mg) and diarrhea (12 AEs in total) were also commonly reported AEs following treatment.
5) There were no clinically significant abnormalities in ECG or vital signs.
6) There were no clinically significant abnormalities identified during gallbladder ultrasound assessments. Biliary sludge was reported for a number of subjects, which was equally distributed between treatment groups. All gallbladders were normal at the post-study examination on Day 70.
7) There appeared to be more incidences of erythema following ATRIGEL®/Octreotide 20 mg compared to Sandostatin LAR® 20 mg. Following dosing of ATRIGEL®/Octreotide 20 mg subjects were completely free of erythema symptoms from 35 days post-dose. Following Sandostatin LAR® 20 mg treatment throughout the study there were always more than 7 subjects absent of erythema symptoms.
8) TSH was below the normal range for 80% of subjects prior to or on Day 3 following ATRIGEL®/Octreotide 20 mg compared to only 20% following Sandostatin LAR® 20 mg. Total T₄ concentrations were below the normal range for 30% of subjects who received ATRIGEL®/Octreotide 20 mg on Day 3 compared to 0% of subjects following Sandostatin LAR® 20 mg. For 4 subjects individual glucagon concentrations declined below the normal range of 50 pg/mL prior to or on Day 7; 30% (3/10) of these subjects received ATRIGEL®/Octreotide 20 mg.
9) On 51 occasions in 15 subjects insulin decreased below the normal range, 57% (29/51) occurring in subjects who received ATRIGEL®/Octreotide 20 mg and 43% (22/51) in subjects who received Sandostatin LAR® 20 mg. On 18 occasions (out of 29) after administration of ATRIGEL®/Octreotide 20 mg and on 10 occasions (out of 22) after administration of Sandostatin LAR® 20 mg insulin decreased below the normal range on or before Day 7. The greater number or earlier events following ATRIGEL®/Octreotide 20 mg may have been related to higher initial concentrations of octreotide after this treatment. All concentrations out of range returned to normal soon afterwards before the post-study assessment and none of the deviations outside the normal range were considered to be clinically significant.
10) Clinical laboratory measurements showed 33 potentially clinically significant changes due to unknown cause that returned to within the normal range upon repeat or soon afterwards before the post-study assessment.

### 6. Pharmacokinetic and Pharmacodynamic Evaluation

### 6.1 Data Sets Analysed

### 6.1.1 Pharmacokinetic Data Sets

The pharmacokinetic analysis population consisted of 20 subjects who were exposed to at least one dose of ATRIGEL®/Octreotide 20 mg or Sandostatin LAR® 20 mg. Subject 8 withdrew following pre-treatment AEs and was replaced by Subject 108.

### 6.1.2 Pharmacodynamic Data Sets

The pharmacodynamic analysis population consisted of 20 subjects who were exposed to at least one dose of ATRIGEL®/Octreotide 20 mg or Sandostatin LAR® 20 mg and for whom samples for IGF-1 assessment were taken up to Day 70. Subject 8 withdrew following pre-treatment AEs and was replaced by Subject 108.

### 6.2 Pharmacokinetic Results

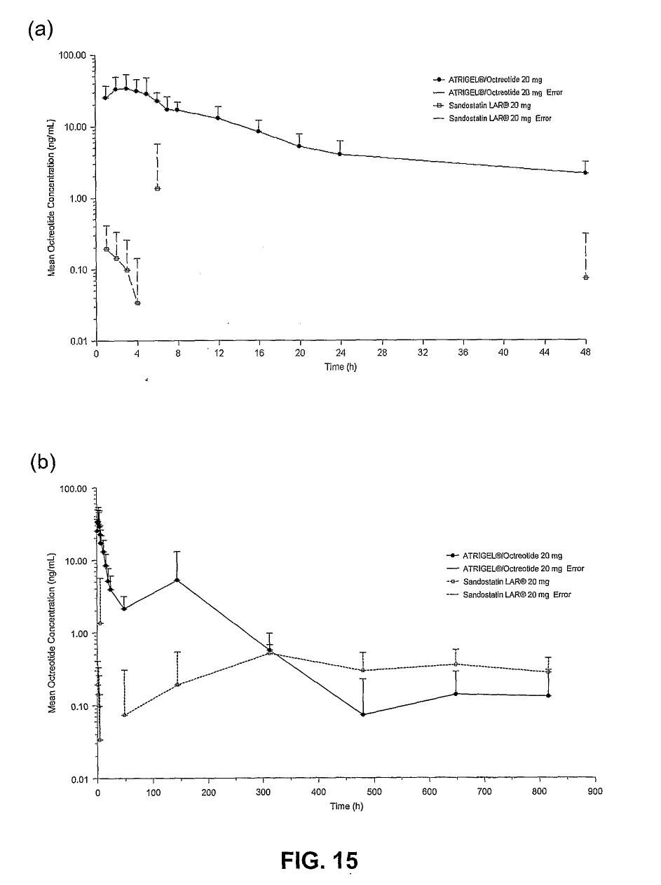

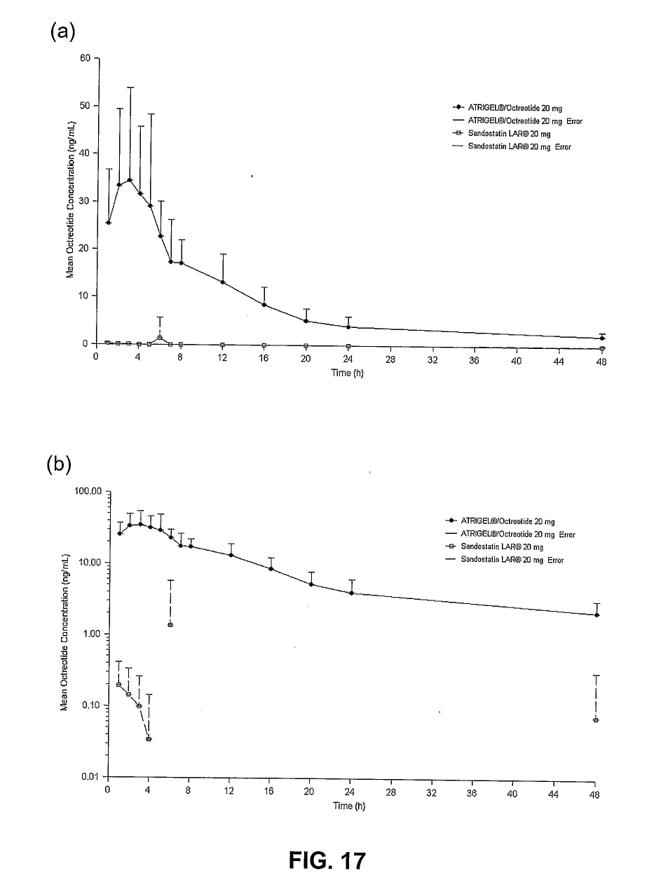

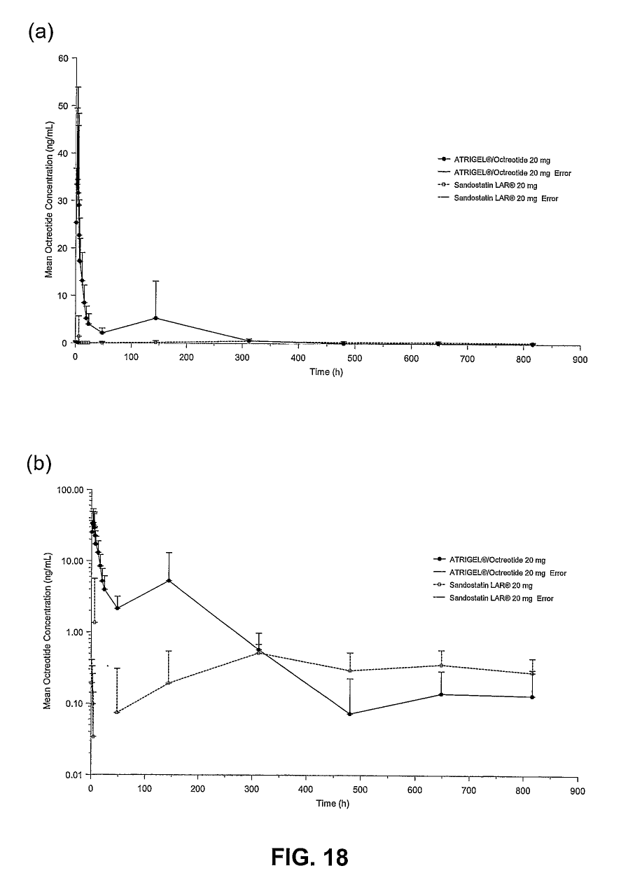

Mean (+SD) log-linear octreotide plasma concentration-time profiles (0-48 hours) and (O-Day 35) are illustrated in FIG. 15 and mean (+SD) linear and log-linear octreotide plasma profiles are illustrated in FIGS. 17 and 18. Individual and summary octreotide pharmacokinetic parameters are summarised in Section 8, Table 6-32.1 and 6-32.2.

After dosing of ATRIGEL®/Octreotide 20 mg there was a rapid rise in mean octreotide plasma concentrations reaching peak levels of approximately 35 ng/mL after a median time of 3 hours before subsequently declining rapidly up to 48 hours and then declining more slowly thereafter. This pattern was consistent with an initial burst or pulse of drug upon administration subsequently followed by slow release of octreotide from the depot site. For most time points mean concentrations for Sandostatin® LAR 20 mg were either BLQ or close to BLQ. The low levels from Day 2 onwards suggest slow release of octreotide into the circulation from the depot site.

Summary pharmacokinetic parameters are given in Table 6-4.

TABLE 6-4
Summary Pharmacokinetic Parameters Following Administration
of Single s.c. Doses of ATRIGEL ®/Octreotide 20 mg and Single i.m.
Doses of Sandostatin LAR ® 20 mg to Separate Groups of Subjects.

| | | ATRIGEL ® 20 mg | Sandostatin |
|---|---|---|---|
| | Summary | s.c. | LAR ® 20 mg i.m. |
| Parameter | Statistics | Depot (n = 10) | Depot (n = 10) |
| t_{lag} (h) | Median | 0.000 | 0.500 |
| | Range | (0.00-0.00) | (0.00-144.00) |
| Cₘₐₓ (ng/mL) | Mean | 38.370 | 1.884 |
| | SD | 21.408 | 4.119 |
| tₘₐₓ (h) | Median | 3.000 | 312.00 |
| | Range | (2.00-5.00) | (6.00-648.17) |
| AUC₀₋₂₄ | Mean | 353.2 | 1.8 |
| (ng · h/mL) | SD | 142.4 | 4.1 |
| AUC₀₋ₜ | Mean | 1351.6 | 253.7 |
| (ng · h/mL) | SD | 953.3 | 104.0 |

There was no lag time in octreotide absorption following ATRIGEL®/Octreotide 20 mg, however there was a median lag time of 0.50 hours and a maximum value of 144.00 hours following treatment of Sandostatin LAR® 20 mg. Mean peak plasma octreotide concentrations of 38.4 and 1.9 ng/mL were achieved at median times of 3.0 and 312.0 hours after dosing of ATRIGEL®/Octreotide 20 mg and Sandostatin LAR® 20 mg, respectively. The greater and earlier value of Cₘₐₓ following ATRIGEL®/Octreotide 20 mg was due to the initial burst/pulse delivery of octreotide. Mean values of AUC₀₋ₜ for ATRIGEL®/Octreotide 20 mg were found to be over 5 times greater than for Sandostatin LAR® 20 mg, indicating the greater exposure to octreotide with the novel product. The effect of the burst of octreotide immediately following injection is shown in the difference between AUC₀-∞₂₄ for the two products, 353.2±142.4 ng.h/mL for ATRIGEL®/Octreotide 20 mg and 1.8±4.1 ng.h/mL for Sandostatin LAR® 20 mg.

### 6.3 Pharmacodynamic Results

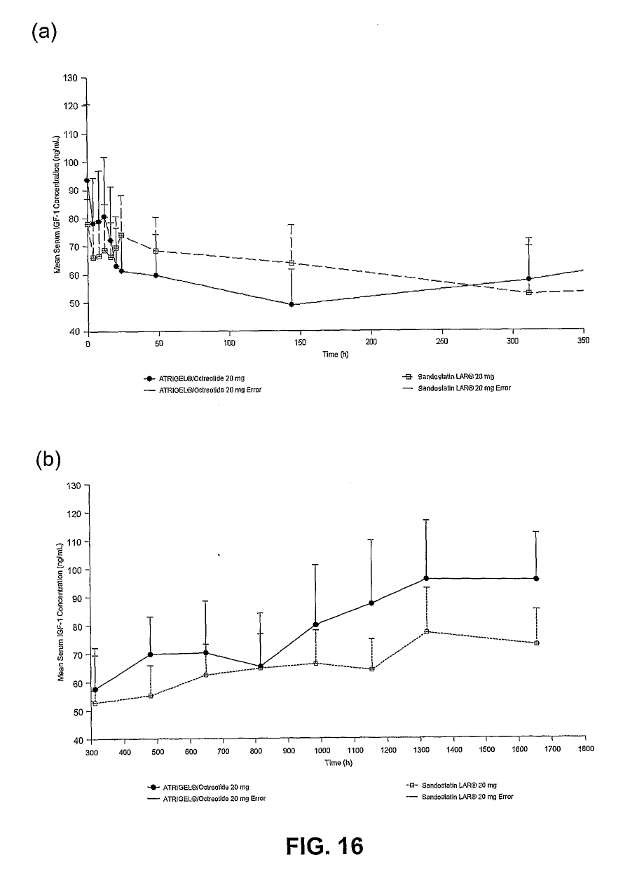

Mean (+SD) serum IGF-1 concentration-time profiles (Day 0-14) and (Day 14-70) are in FIG. 16.

Following ATRIGEL®/Octreotide 20 mg dosing, mean IGF-1 concentrations declined from a pre-dose value of ≈95 ng/mL to ≈50 ng/mL (≈50% change from baseline) on Day 7, mean concentrations subsequently increased steadily and returned close to pre-dose levels on Day 49. Mean IGF-1 concentrations following dosing of Sandostatin LAR® 20 mg declined from ≈80 ng/mL pre-dose to a minimum level of ≈50 ng/mL (≈38% change from baseline) on Day 14 before returning close to pre-dose levels on Day 56. The inhibition of IGF-1 secretion appeared to be greater in magnitude following ATRIGEL®/Octreotide 20 mg compared to Sandostatin LAR® 20 mg.

### 6.4 Statistical Results

The statistical analysis comparison of the pharmacokinetic parameters Cₘₐₓ, AUC₀₋ₜ and tₘₐₓ for ATRIGEL®/Octreotide 20 mg and Sandostatin LAR® 20 mg are given below in Table 6-5.

TABLE 6-5
Point Estimates and 90% CI for the Treatment Comparisons of
ATRIGEL ®/Octreotide 20 mg vs. Sandostatin LAR ® 20 mg Pharmacokinetic
Comparison Populations.

| | | LS Means | | | | | |
|---|---|---|---|---|---|---|---|
| | | Test | Reference | | | | |
| | | ATRIGEL/ | Sandostatin | Point | 90% CI | | |
| Parameter | n | Octreotide 20 mg | LAR 20 mg | Estimate(%) | Lower | Upper | p-value |
| Cₘₐₓ | 10 | 34.22 | 0.77 | 4443.82 | 2349.80 | 8403.94 | <0.001 |
| AUC₀₋ₜ | 10 | 1186.02 | 212.07 | 559.26 | 331.99 | 942.11 | <0.001 |

Sum of Scores

| | | | Reference | |
|---|---|---|---|---|
| | | Test ATRIGEL ®/ | Sandostatin | Wilcoxon Exact Test |
| Parameter | n | Octreotide 20 mg | LAR ® 20 mg | p-value |
| tₘₐₓ | 10 | 55 | 155 | <0.0001 |

Based on 90% CI treatment comparisons of Cₘₐₓ and AUC₀₋ₜ, dosing of ATRIGEL®/Octreotide 20 mg resulted in a statistcically significantly greater exposure to octreotide compared to Sandostatin LAR® 20 mg treatment. The tₘₐₓ was found to occur statistically significantly earlier for ATRIGEL®/Octreotide 20 mg compared to Sandostatin LAR® 20 mg.

The statistical analysis comparison for the pharmacodynamic parameter IGF-1 at each timepoint for ATRIGEL®/Octreotide 20 mg and Sandostatin LAR® 20 mg is given in Table 6-6.

TABLE 6-6
Point Estimates and 90% CI for the Treatment Comparisons of
Serum IGF-1 Concentrations Following Administration of
ATRIGEL ®/Octreotide 20 mg vs. Sandostatin LAR ® 20 mg (n = 10).

| | LS Means | | | | | |
|---|---|---|---|---|---|---|
| | (ATRIGEL/ | (Sandostatin | Point Estimate | 90% CI | | |
| Timepoint | Octreotide 20 mg) | LAR 20 mg) | (%) | Lower | Upper | P-value |
| Pre-dose | 90.6 | 77.7 | 116.63 | 99.37 | 136.87 | 0.1130 |
| Day 1, 4 h | 76.7 | 65.1 | 117.81 | 100.52 | 138.06 | 0.0901 |
| Day 1, 8 h | 77.0 | 65.5 | 117.63 | 99.62 | 138.90 | 0.1074 |
| Day 1, 12 h | 78.2 | 67.2 | 116.32 | 96.25 | 140.57 | 0.1832 |
| Day 1, 16 h | 70.3 | 65.4 | 107.50 | 90.76 | 127.32 | 0.4684 |
| Day 1, 20 h | 61.8 | 68.8 | 89.88 | 77.94 | 103.66 | 0.2111 |
| Day 1, 24 h | 60.2 | 72.9 | 82.52 | 71.17 | 95.68 | 0.0370 |
| Day 3 | 58.3 | 67.6 | 86.14 | 73.16 | 101.43 | 0.1309 |
| Day 7 | 47.6 | 62.5 | 76.10 | 63.30 | 91.49 | 0.0192 |
| Day 14 | 56.1 | 49.8 | 112.76 | 87.55 | 145.22 | 0.4215 |
| Day 21 | 68.9 | 54.3 | 126.99 | 109.28 | 147.58 | 0.0129 |
| Day 28 | 67.9 | 61.6 | 110.25 | 91.81 | 132.38 | 0.3675 |
| Day 35 | 62.4 | 63.8 | 97.84 | 79.14 | 120.96 | 0.8604 |
| Day 42 | 77.3 | 65.3 | 118.46 | 98.44 | 142.56 | 0.1300 |
| Day 49 | 85.0¹ | 63.2 | 134.50 | 113.34 | 159.61 | 0.0078 |
| Day 56 | 93.9 | 75.8 | 123.98 | 105.12 | 146.23 | 0.0365 |
| Day 70 | 94.4 | 71.9 | 131.15 | 114.46 | 150.26 | 0.0028 |

¹n = 9, Statistically significant differences are highlighted.

After administration of ATRIGEL®/Octreotide 20 mg, IGF-1 levels were statistically significantly greater than following the reference therapy (Sandostatin LAR® 20 mg) at nominal times of 4 hours and on Days 21, 49, 56 and 70. At 24 hours and on Day 7 following administration of ATRIGEL®/Octreotide 20 mg, IGF-1 levels were statistically significantly lower than compared to IGF-1 levels following Sandostatin LAR® 20 mg. All other statistical comparisons were inconclusive based on the conventional confidence interval approach. The 90% confidence intervals exceeded either the lower or upper limit set by the FDA for bioequivalence testing [80 to 125%].

### 7. Discussion and Overall Conclusions

This was a Phase 1, randomized, single dose, open-label comparative study of the pharmacokientics, pharmacodynamics and safety of ATRIGEL®/Octreotide 1-month depot and Sandostatin LAR® 1-month depot 20 mg in healthy male subjects. The main objectives of the study were to compare the pharmacokinetic and pharmacodynamic characteristics and to assess the safety and tolerability of ATRIGEL®/Octreotide 20 mg and Sandostatin LAR® 20 mg. Pharmacokinetics and pharmacodynamics were assessed by plasma concentrations of octreotide and IGF-1, respectively, and safety was determined through plasma levels of TSH, total T₄, free T₄, fasting glucose and insulin, glucagon, gallbladder ultrasounds, ECGs, clinical lab results, vital signs and monitoring of adverse events.

In general, the administration of both ATRIGEL®/Octreotide and Sandostatin LAR® was well tolerated by the subjects in the study. None of the adverse events required treatment other than observation, or sometimes the administration of concomitant medication. Some subjects required more prolonged follow up beyond the scheduled post-study medical to ensure full resolution of adverse events. There was a total of 101 treatment-emergent adverse events reported by 19 subjects. Sixty-one AEs (60.4% of total) in 10 subjects were observed following s.c. ATRIGEL® 20 mg depot treatment and 40 AEs (39.6% of total) in 9 subjects following Sandostatin LAR® 20 mg i.m. depot administration. Subject 19 did not report any AEs throughout the study.

The most common AEs were related to the injection site. There were 23 cases of erythema in 16 subjects at the injection site which ranged from mild to moderate intensity. Fifteen cases in 10 subjects followed ATRIGEL®/Octreotide 20 mg, compared to 8 events in 6 subjects following Sandostatin LAR® 20 mg. Most AEs following ATRIGEL®/Octreotide 20 mg were moderate in intensity (10 AEs in 10 subjects) compared to a single moderate intensity AE with Sandostatin LAR® 20 mg. Ten subjects also reported palpable masses at the injection site, most were reported following ATRIGEL®/Octreotide 20 mg (9 AEs in 8 subjects). Adverse events related to local tolerability of the injection were not associated with any systemic upset. The higher frequency of local tolerability AEs at the injection site following s.c. ATRIGEL®/Octreotide 20 mg compared to i.m. Sandostatin LAR® 20 mg may have been related to the mode and location of administration.

Other commonly reported AEs were related to gastrointestinal effects (diarrhea, abdominal discomfort, abdominal distension, flatulence and loose stools). These were as predicted from previous studies with octreotide and similar for both treatments, (12 AEs in 6 subjects) and (11 AEs in 7 subjects), following ATRIGEL®/Octreotide 20 mg and Sandostatin LAR® 20 mg, respectively. Other systemic AEs also appeared to be similar in frequency for both treatments.

There were no clinically significant abnormalities in ECG, vital signs, gallbladder ultrasound assessment, injection site assessment and endocrine profiles. Clinical laboratory measurements showed 35 potentially clinically significant changes due to unknown cause that returned to normal upon repeat or soon afterwards by the end of dosing or post medical at the latest. Gallbladder ultrasound assessments revealed the presence of biliary sludge in a similar number of subjects in each treatment group. However all gallbladders were normal at the post-study examination on Day 70. Injection site assessment showed that following dosing of ATRIGEL®/Octreotide 20 mg subjects were completely free of erythema symptoms after 35 days and for Sandostatin LAR® 20 mg treatment there were always more than 7 subjects absent of erythema symptoms.

For some subjects individual concentrations of TSH, total T₄ and free T₄ were observed to be below the normal range particularly at the early times following dosing of ATRIGEL®/Octreotide 20 mg. For 4 subjects (3 of which received ATRIGEL®/Octreotide 20 mg) glucagon decreased below the normal range. In subjects who received ATRIGEL®/Octreotide 20 mg, glucagon was below the normal range on or prior to Day 7 on ¾ subjects; this may have been dependent on the initial burst of octreotide from this treatment. On 51 occasions in 15 subjects insulin decreased below the normal range, 57% (29/51) occurring in subjects who received ATRIGEL®/Octreotide 20 mg and 43% (22/51) in subjects who received Sandostatin LAR® 20 mg. All concentrations out of range returned to normal soon afterwards before the post-study assessment. The effects on endocrine profiles were predicted from previous studies with octreotide and none of the deviations outside the normal range were considered to be clinically significant.

Mean peak plasma octreotide concentrations of 38.4 and 1.9 ng/mL were achieved at median times of 3.0 and 312.0 hours after dosing of ATRIGEL®/Octreotide 20 mg and Sandostatin LAR® 20 mg, respectively. The difference in magnitude and time of Cₘₐₓ following ATRIGEL®/Octreotide 20 mg was due to the initial burst/pulse delivery of octreotide. The effect of this was shown in the comparison of values of AUC₀₋₂₄ which was nearly 200 fold higher for ATRIGEL®/Octreotide 20 mg. Mean values of AUC₀₋ₜ for ATRIGEL®/Octreotide 20 mg were found to be over 5 times greater than for Sandostatin LAR® 20 mg, indicating the improved degree of exposure to octreotide with the novel product. Exposure to octreotide following ATRIGEL®/Octreotide 20 mg was found to be statistcically significantly greater compared to Sandostatin LAR® 20 mg based on Cₘₐₓ and AUC₀₋ₜ comparisons.

Mean IGF-1 concentrations following dosing of ATRIGEL®/Octreotide 20 mg declined by approximately 45 ng/mL from baseline (≈50% change from baseline) to Day 7 before returning close to pre-dose levels on Day 49. Mean IGF-1 concentrations following dosing of Sandostatin LAR® 20 mg declined by approximately 30 ng/mL from baseline (˜38% change from baseline) to Day 14 before returning close to pre-dose levels on Day 56. The inhibition of IGF-1 secretion appeared to be greater in magnitude following ATRIGEL®/Octreotide 20 mg compared to Sandostatin LAR® 20 mg. The greater effect of ATRIGEL® on IGF-1 release may be related to the initial burst of octreotide following injection of the novel device. At most timepoints statistical comparisons of IGF-1 levels were inconclusive based on the conventional confidence interval approach. These results suggested that the sample size was not large enough to allow definitive conclusions to be drawn regarding the level of IGF-1 observed with the two treatments.

### Final Conclusions

In conclusion, in general the treatments were well tolerated. There were no deaths or serious adverse events during the study. There was a total of 101 treatment-emergent AEs, 80 of which were mild and 21 moderate in intensity. Sixty-one AEs in 10 subjects were observed following ATRIGEL®/Octreotide 20 mg and 40 AEs in 9 subjects following Sandostatin LAR® 20 mg. Most common reported AEs were injection site erythema, injection site nodule and diarrhea.

Fifty-two AEs were “certainly, possibly or probably related” to ATRIGEL®/Octreotide 20 mg and 28 AEs were “certainly, possibly or probably related” to Sandostatin LAR® 20 mg. Injection site erythema was the most common reported AEs with 23 treatment related occurrences in 16 subjects, 15 of these events in 10 subjects occurred following ATRIGEL®/Octreotide 20 mg. Injection site nodule (12 AEs in total) (9 in 8 subjects following ATRIGEL®/Octreotide 20 mg) and diarrhea (12 AEs in total) were also commonly reported AEs following treatment. Following ATRIGEL®/Octreotide 20 mg, subjects were completely free of erythema from 35 days post-dose. Throughout the study there were always more than 7 subjects absent of erythema symptoms following Sandostatin LAR® 20 mg. There were no clinically significant abnormalities in ECG or vital signs.

There were no clinically significant abnormalities identified during gallbladder ultrasound assessments. Biliary sludge was reported for a number of subjects, however all gallbladders were normal at the post-study examination on Day 70.

TSH was below the normal range for 80% of subjects prior to or on Day 3 following ATRIGEL®/Octreotide 20 mg compared to only 20% following Sandostatin LAR® 20 mg. Total T₄ concentrations were below the normal range for 30% of subjects who received ATRIGEL®/Octreotide 20 mg on Day 3 compared to 0% of subjects following Sandostatin LAR® 20 mg. For 4 subjects individual glucagon concentrations declined below the normal range of 50 pg/mL prior to or on Day 7; 30% (3/10) of these subjects received ATRIGEL®/Octreotide 20 mg.

On 51 occasions in 15 subjects insulin decreased below the normal range, 57% (29/51) occurring in subjects who received ATRIGEL®/Octreotide 20 mg and 43% (22/51) in subjects who received Sandostatin LAR® 20 mg. On 18 occasions (out of 29) after administration of ATRIGEL®/Octreotide 20 mg and on 10 occasions (out of 22) after administration of Sandostatin LAR® 20 mg insulin decreased below the normal range on or before Day 7. The greater number or earlier events following ATRIGEL®/Octreotide 20 mg may have been related to higher initial concentrations of octreotide after this treatment. All concentrations out of range returned to normal soon afterwards before the post-study assessment and none of the deviations outside the normal range were considered to be clinically significant.

Clinical laboratory measurements showed 33 potentially clinically significant changes due to unknown cause that returned to within the normal range upon repeat or soon afterwards before the post-study assessment.

High (38.4 ng/mL) and early (3.0 h) values of Cₘₐₓ following ATRIGEL®/Octreotide 20 mg were due to the initial burst/pulse delivery of octreotide at administration. The effect of this was shown in the comparison of values of AUC₀₋₂₄ which was nearly 200 fold higher for ATRIGEL®/Octreotide 20 mg treatments compared to Sandostatin LARD 20 mg treatments.

Mean values of AUC₀₋ₜ for ATRIGEL®/Octreotide 20 mg were almost 5 fold higher than for Sandostatin LAR® 20 mg, indicating the improved degree of overall exposure to octreotide with the novel product. Exposure to octreotide following ATRIGEL®/Octreotide 20 mg was found to be statistcically significantly greater compared to Sandostatin LAR® 20 mg based on comparisons of Cₘₐₓ and AUC₀₋ₜ and tₘₐₓ was also found to occur statistically significantly earlier for ATRIGEL®/Octreotide 20 mg.

Mean IGF-1 concentrations following dosing of ATRIGEL®/Octreotide 20 mg declined by a greater magnitude from baseline (approximately 12%) and more rapidly (7 Days) compared to IGF-1 concentrations following Sandostatin LAR® 20 mg. The greater effect of ATRIGEL® on IGF-1 may be due to the initial burst of octreotide. However, at most timepoints statistical comparisons of IGF-1 levels were inconclusive suggesting the sample size was not large enough to allow definitive conclusions regarding the level of IGF-1 observed with the two treatments.

### 8. Tabulated Data

### 8.1 Tables for Example 6

Clinical Laboratory Values Summary Statistics

TABLE 6-7.1
Summary of Hematology Parameters
Summary of Hematology Laboratory Values
Safety Population

| Laboratory Test | Units | Visit (Day) | Treatment | Summary Statistic | Value |
|---|---|---|---|---|---|
| Basophils | E + 9/L | Screening | ATRIGEL ®/Octreotide 20 mg | n | 10 |
| | | | | Mean | 0.06 |
| | | | | SD | 0.03 |
| | | | | CV % | 46.1 |
| | | | | Median | 0.06 |
| | | Day−1 | ATRIGEL ®/Octreotide 20 mg | n | 10 |
| | | | | Mean | 0.04 |
| | | | | SD | 0.01 |
| | | | | CV % | 34.8 |
| | | | | Median | 0.04 |
| | | Day 2 | ATRIGEL ®/Octreotide 20 mg | n | 10 |
| | | | | Mean | 0.07 |
| | | | | SD | 0.07 |
| | | | | CV % | 95.5 |
| | | | | Median | 0.05 |
| | | Day 28 | ATRIGEL ®/Octreotide 20 mg | n | 10 |
| | | | | Mean | 0.05 |
| | | | | SD | 0.02 |
| | | | | CV % | 40.2 |
| | | | | Median | 0.05 |
| | | Day 70 | ATRIGEL ®/Octreotide 20 mg | n | 10 |
| | | | | Mean | 0.06 |
| | | | | SD | 0.02 |
| | | | | CV % | 40.8 |
| | | | | Median | 0.06 |
| Eosinophils | E + 9/L | Screening | ATRIGEL ®/Octreotide 20 mg | n | 10 |
| | | | | Mean | 0.19 |
| | | | | SD | 0.11 |
| | | | | CV % | 54.7 |
| | | | | Median | 0.15 |
| | | Day−1 | ATRIGEL ®/Octreotide 20 mg | n | 10 |
| | | | | Mean | 0.16 |
| | | | | SD | 0.14 |
| | | | | CV % | 89.7 |
| | | | | Median | 0.12 |
| | | Day 2 | ATRIGEL ®/Octreotide 20 mg | n | 10 |
| | | | | Mean | 0.22 |
| | | | | SD | 0.09 |
| | | | | CV % | 40.5 |
| | | | | Median | 0.21 |
| | | Day 28 | ATRIGEL ®/Octreotide 20 mg | n | 10 |
| | | | | Mean | 0.24 |
| | | | | SD | 0.13 |
| | | | | CV % | 53.5 |
| | | | | Median | 0.19 |
| | | Day 70 | ATRIGEL ®/Octreotide 20 mg | n | 10 |
| | | | | Mean | 0.23 |
| | | | | SD | 0.11 |
| | | | | CV % | 47.1 |
| | | | | Median | 0.20 |
| Haemoglobin | g/dL | Screening | ATRIGEL ®/Octreotide 20 mg | n | 10 |
| | | | | Mean | 14.90 |
| | | | | 50 | 0.75 |
| | | | | CV % | 5.0 |
| | | | | Median | 14.75 |
| | | Day−1 | ATRIGEL ®/Octreotide 20 mg | n | 10 |
| | | | | Mean | 14.69 |
| | | | | SD | 0.87 |
| | | | | CV % | 5.9 |
| | | | | Median | 14.90 |
| | | Day 2 | ATRIGEL ®/Octreotide 20 mg | n | 10 |
| | | | | Mean | 15.61 |
| | | | | SD | 1.05 |
| | | | | CV % | 6.8 |
| | | | | Median | 15.55 |
| | | Day 28 | ATRIGEL ®/Octreotide 20 mg | n | 10 |
| | | | | Mean | 14.11 |
| | | | | SD | 0.86 |
| | | | | CV % | 6.1 |
| | | | | Median | 14.35 |
| | | Day 70 | ATRIGEL ®/Octreotide 20 mg | n | 10 |
| | | | | Mean | 14.32 |
| | | | | SD | 0.99 |
| | | | | CV % | 6.9 |
| | | | | Median | 14.50 |
| Lymphocytes | E + 9/L | Screening | ATRIGEL ®/Octreotide 20 mg | n | 10 |
| | | | | Mean | 1.93 |
| | | | | SD | 0.31 |
| | | | | CV % | 16.2 |
| | | | | Median | 1.86 |
| | | Day−1 | ATRIGEL ®/Octreotide 20 mg | n | 10 |
| | | | | Mean | 1.72 |
| | | | | SD | 0.46 |
| | | | | CV % | 26.9 |
| | | | | Median | 1.65 |
| | | Day 2 | ATRIGEL ®/Octreotide 20 mg | n | 10 |
| | | | | Mean | 1.67 |
| | | | | SD | 0.55 |
| | | | | CV % | 32.9 |
| | | | | Median | 1.60 |
| | | Day 28 | ATRIGEL ®/Octreotide 20 mg | n | 10 |
| | | | | Mean | 2.11 |
| | | | | SD | 0.56 |
| | | | | CV % | 26.7 |
| | | | | Median | 1.93 |
| | | Day 70 | ATRIGEL ®/Octreotide 20 mg | n | 10 |
| | | | | Mean | 1.86 |
| | | | | SD | 0.45 |
| | | | | CV % | 24.4 |
| | | | | Median | 1.82 |
| MCH | pg | Screening | ATRIGEL ®/Octreotide 20 mg | n | 10 |
| | | | | Mean | 30.80 |
| | | | | SD | 1.25 |
| | | | | CV % | 4.1 |
| | | | | Median | 30.55 |
| | | Day−1 | ATRIGEL ®/Octreotide 20 mg | n | 10 |
| | | | | Mean | 30.14 |
| | | | | SD | 1.23 |
| | | | | CV % | 4.1 |
| | | | | Median | 29.80 |
| | | Day 2 | ATRIGEL ®/Octreotide 20 mg | n | 10 |
| | | | | Mean | 30.49 |
| | | | | SD | 1.30 |
| | | | | CV % | 4.3 |
| | | | | Median | 30.20 |
| | | Day 28 | ATRIGEL ®/Octreotide 20 mg | n | 10 |
| | | | | Mean | 30.16 |
| | | | | SD | 1.16 |
| | | | | CV % | 3.9 |
| | | | | Median | 29.95 |
| | | Day 70 | ATRIGEL ®/Octreotide 20 mg | n | 10 |
| | | | | Mean | 30.03 |
| | | | | SD | 1.13 |
| | | | | CV % | 3.8 |
| | | | | Median | 29.90 |
| MCHC | g/dL | Screening | ATRIGEL ®/Octreotide 20 mg | n | 10 |
| | | | | Mean | 33.42 |
| | | | | SD | 0.46 |
| | | | | CV % | 1.4 |
| | | | | Median | 33.45 |
| | | Day−1 | ATRIGEL ®/Octreotide 20 mg | n | 10 |
| | | | | Mean | 33.40 |
| | | | | SD | 0.68 |
| | | | | CV % | 2.0 |
| | | | | Median | 33.35 |
| | | Day 2 | ATRIGEL ®/Octreotide 20 mg | n | 10 |
| | | | | Mean | 33.85 |
| | | | | SD | 0.73 |
| | | | | CV % | 2.2 |
| | | | | Median | 33.90 |
| | | Day 28 | ATRIGEL ®/Octreotide 20 mg | n | 10 |
| | | | | Mean | 33.14 |
| | | | | SD | 0.58 |
| | | | | CV % | 1.8 |
| | | | | Median | 33.05 |
| | | Day 70 | ATRIGEL ®/Octreotide 20 mg | n | 10 |
| | | | | Mean | 32.94 |
| | | | | SD | 0.74 |
| | | | | CV % | 2.3 |
| | | | | Median | 32.60 |
| Mean Cell Volume | fL | Screening | ATRIGEL ®/Octreotide 20 mg | n | 10 |
| | | | | Mean | 92.21 |
| | | | | SD | 4.23 |
| | | | | CV % | 4.6 |
| | | | | Median | 90.80 |
| | | Day−1 | ATRIGEL ®/Octreotide 20 mg | n | 10 |
| | | | | Mean | 90.32 |
| | | | | SD | 4.85 |
| | | | | CV % | 5.4 |
| | | | | Median | 89.10 |
| | | Day 2 | ATRIGEL ®/Octreotide 20 mg | n | 10 |
| | | | | Mean | 90.14 |
| | | | | SD | 4.68 |
| | | | | CV % | 5.2 |
| | | | | Median | 88.75 |
| | | Day 28 | ATRIGEL ®/Octreotide 20 mg | n | 10 |
| | | | | Mean | 91.04 |
| | | | | SD | 4.30 |
| | | | | CV % | 4.7 |
| | | | | Median | 90.30 |
| | | Day 70 | ATRIGEL ®/Octreotide 20 mg | n | 10 |
| | | | | Mean | 91.18 |
| | | | | SD | 4.32 |
| | | | | CV % | 4.7 |
| | | | | Median | 89.85 |
| Monocytes | E + 9/L | Screening | ATRIGEL ®/Octreotide 20 mg | n | 10 |
| | | | | Mean | 0.56 |
| | | | | SD | 0.14 |
| | | | | CV % | 25.2 |
| | | | | Median | 0.53 |
| | | Day−1 | ATRIGEL ®/Octreotide 20 mg | n | 10 |
| | | | | Mean | 0.48 |
| | | | | SD | 0.19 |
| | | | | CV % | 39.9 |
| | | | | Median | 0.42 |
| | | Day 2 | ATRIGEL ®/Octreotide 20 mg | n | 10 |
| | | | | Mean | 0.50 |
| | | | | SD | 0.21 |
| | | | | CV % | 41.5 |
| | | | | Median | 0.45 |
| | | Day 28 | ATRIGEL ®/Octreotide 20 mg | n | 10 |
| | | | | Mean | 0.49 |
| | | | | SD | 0.18 |
| | | | | CV % | 37.3 |
| | | | | Median | 0.49 |
| | | Day 70 | ATRIGEL ®/Octreotide 20 mg | n | 10 |
| | | | | Mean | 0.55 |
| | | | | SD | 0.20 |
| | | | | CV % | 36.6 |
| | | | | Median | 0.52 |
| Neutrophils | E + 9/L | Screening | ATRIGEL ®/Octreotide 20 mg | n | 10 |
| | | | | Mean | 3.22 |
| | | | | SD | 1.06 |
| | | | | CV % | 32.8 |
| | | | | Median | 3.13 |
| | | Day−1 | ATRIGEL ®/Octreotide 20 mg | n | 10 |
| | | | | Mean | 2.80 |
| | | | | SD | 1.07 |
| | | | | CV % | 38.3 |
| | | | | Median | 2.53 |
| | | Day 2 | ATRIGEL ®/Octreotide 20 mg | n | 10 |
| | | | | Mean | 3.44 |
| | | | | SD | 1.02 |
| | | | | CV % | 29.8 |
| | | | | Median | 2.97 |
| | | Day 28 | ATRIGEL ®/Octreotide 20 mg | n | 10 |
| | | | | Mean | 3.17 |
| | | | | SD | 0.74 |
| | | | | CV % | 23.5 |
| | | | | Median | 3.17 |
| | | Day 70 | ATRIGEL ®/Octreotide 20 mg | n | 10 |
| | | | | Mean | 3.71 |
| | | | | SD | 1.90 |
| | | | | CV % | 51.3 |
| | | | | Median | 2.85 |
| Packed Cell Volume | | Screening | ATRIGEL ®/Octreotide 20 mg | n | 10 |
| | | | | Mean | 0.45 |
| | | | | SD | 0.02 |
| | | | | CV % | 4.8 |
| | | | | Median | 0.45 |
| | | Day−1 | ATRIGEL ®/Octreotide 20 mg | n | 10 |
| | | | | Mean | 0.44 |
| | | | | SD | 0.03 |
| | | | | CV % | 6.3 |
| | | | | Median | 0.45 |
| | | Day 2 | ATRIGEL ®/Octreotide 20 mg | n | 10 |
| | | | | Mean | 0.46 |
| | | | | SD | 0.03 |
| | | | | CV % | 7.0 |
| | | | | Median | 0.47 |
| | | Day 28 | ATRIGEL ®/Octreotide 20 mg | n | 10 |
| | | | | Mean | 0.43 |
| | | | | SD | 0.03 |
| | | | | CV % | 6.6 |
| | | | | Median | 0.43 |
| | | Day 70 | ATRIGEL ®/Octreotide 20 mg | n | 10 |
| | | | | Mean | 0.44 |
| | | | | SD | 0.03 |
| | | | | CV % | 6.0 |
| | | | | Median | 0.44 |
| Platelet Count | E + 9/L | Screening | ATRIGEL ®/Octreotide 20 mg | n | 10 |
| | | | | Mean | 243.3 |
| | | | | SD | 54.39 |
| | | | | CV % | 22.4 |
| | | | | Median | 242.5 |
| | | Day−1 | ATRIGEL ®/Octreotide 20 mg | n | 10 |
| | | | | Mean | 217.4 |
| | | | | SD | 56.01 |
| | | | | CV % | 25.8 |
| | | | | Median | 241.5 |
| | | Day 2 | ATRIGEL ®/Octreotide 20 mg | n | 9 |
| | | | | Mean | 212.2 |
| | | | | SD | 64.12 |
| | | | | CV % | 30.2 |
| | | | | Median | 209.0 |
| | | Day 28 | ATRIGEL ®/Octreotide 20 mg | n | 10 |
| | | | | Mean | 200.3 |
| | | | | SD | 49.19 |
| | | | | CV % | 24.6 |
| | | | | Median | 181.0 |
| | | Day 70 | ATRIGEL ®/Octreotide 20 mg | n | 10 |
| | | | | Mean | 220.0 |
| | | | | SD | 40.77 |
| | | | | CV % | 18.5 |
| | | | | Median | 227.5 |
| Red Cell Count | E + 12/L | Screening | ATRIGEL ®/Octreotide 20 mg | n | 10 |
| | | | | Mean | 4.84 |
| | | | | SD | 0.33 |
| | | | | CV % | 6.9 |
| | | | | Median | 4.85 |
| | | Day−1 | ATRIGEL ®/Octreotide 20 mg | n | 10 |
| | | | | Mean | 4.88 |
| | | | | SD | 0.35 |
| | | | | CV % | 7.1 |
| | | | | Median | 4.85 |
| | | Day 2 | ATRIGEL ®/Octreotide 20 mg | n | 10 |
| | | | | Mean | 5.12 |
| | | | | SD | 0.33 |
| | | | | CV % | 6.4 |
| | | | | Median | 5.20 |
| | | Day 28 | ATRIGEL ®/Octreotide 20 mg | n | 10 |
| | | | | Mean | 4.68 |
| | | | | SD | 0.25 |
| | | | | CV % | 5.3 |
| | | | | Median | 4.73 |
| | | Day 70 | ATRIGEL ®/Octreotide 20 mg | n | 10 |
| | | | | Mean | 4.78 |
| | | | | SD | 0.29 |
| | | | | CV % | 6.1 |
| | | | | Median | 4.77 |
| White Cell Count | E+9/L | Screening | ATRIGEL ®/Octreotide 20 mg | n | 10 |
| | | | | Mean | 5.96 |
| | | | | SD | 1.29 |
| | | | | CV % | 21.7 |
| | | | | Median | 6.10 |
| | | Day−1 | ATRIGEL ®/Octreotide 20 mg | n | 10 |
| | | | | Mean | 5.20 |
| | | | | SD | 1.28 |
| | | | | CV % | 24.6 |
| | | | | Median | 4.75 |
| | | Day 2 | ATRIGEL ®/Octreotide 20 mg | n | 10 |
| | | | | Mean | 5.90 |
| | | | | SD | 1.33 |
| | | | | CV % | 22.6 |
| | | | | Median | 5.55 |
| | | Day 28 | ATRIGEL ®/Octreotide 20 mg | n | 10 |
| | | | | Mean | 6.05 |
| | | | | SD | 1.30 |
| | | | | CV % | 21.5 |
| | | | | Median | 6.35 |
| | | Day 70 | ATRIGEL ®/Octreotide 20 mg | n | 10 |
| | | | | Mean | 6.41 |
| | | | | SD | 2.08 |
| | | | | CV % | 32.4 |
| | | | | Median | 6.10 |

TABLE 6-8.1
Summary of Hematology Laboratory Values
Safety Population

| Laboratory Test | Units | Visit (Day) | Treatment | Summary Statistic | Value |
|---|---|---|---|---|---|
| Basophils | E+9/L | Screening | Sandostatin LAR ® 20 mg | n | 10 |
| | | | | Mean | 0.05 |
| | | | | SD | 0.02 |
| | | | | CV % | 42.9 |
| | | | | Median | 0.06 |
| | | Day−1 | Sandostatin LAR ® 20 mg | n | 10 |
| | | | | Mean | 0.05 |
| | | | | SD | 0.02 |
| | | | | CV % | 35.3 |
| | | | | Median | 0.05 |
| | | Day 2 | Sandostatin LAR ® 20 mg | n | 10 |
| | | | | Mean | 0.07 |
| | | | | SD | 0.03 |
| | | | | CV % | 50.6 |
| | | | | Median | 0.07 |
| | | Day 28 | Sandostatin LAR ® 20 mg | n | 10 |
| | | | | Mean | 0.06 |
| | | | | SD | 0.01 |
| | | | | CV % | 19.8 |
| | | | | Median | 0.06 |
| | | Day 70 | Sandostatin LAR ® 20 mg | n | 10 |
| | | | | Mean | 0.06 |
| | | | | SD | 0.02 |
| | | | | CV % | 27.5 |
| | | | | Median | 0.06 |
| Eosinophils | E+9/L | Screening | Sandostatin LAR ® 20 mg | n | 10 |
| | | | | Mean | 0.16 |
| | | | | SD | 0.08 |
| | | | | CV % | 52.1 |
| | | | | Median | 0.16 |
| | | Day−1 | Sandostatin LAR ® 20 mg | n | 10 |
| | | | | Mean | 0.14 |
| | | | | SD | 0.06 |
| | | | | CV % | 41.0 |
| | | | | Median | 0.13 |
| | | Day 2 | Sandostatin LAR ® 20 mg | n | 10 |
| | | | | Mean | 0.17 |
| | | | | SD | 0.08 |
| | | | | CV % | 46.8 |
| | | | | Median | 0.15 |
| | | Day 28 | Sandostatin LAR ® 20 mg | n | 10 |
| | | | | Mean | 0.27 |
| | | | | SD | 0.18 |
| | | | | CV % | 64.3 |
| | | | | Median | 0.19 |
| | | Day 70 | Sandostatin LAR ® 20 mg | n | 10 |
| | | | | Mean | 0.19 |
| | | | | SD | 0.07 |
| | | | | CV % | 37.3 |
| | | | | Median | 0.21 |
| Haemoglobin | g/dL | Screening | Sandostatin LAR ® 20 mg | n | 10 |
| | | | | Mean | 15.51 |
| | | | | SD | 1.02 |
| | | | | CV % | 6.6 |
| | | | | Median | 15.50 |
| | | Day−1 | Sandostatin LAR ® 20 mg | n | 10 |
| | | | | Mean | 15.28 |
| | | | | SD | 1.04 |
| | | | | CV % | 6.8 |
| | | | | Median | 15.35 |
| | | Day 2 | Sandostatin LAR ® 20 mg | n | 10 |
| | | | | Mean | 16.22 |
| | | | | SD | 0.87 |
| | | | | CV % | 5.4 |
| | | | | Median | 16.30 |
| | | Day 28 | Sandostatin LAR ® 20 mg | n | 10 |
| | | | | Mean | 14.72 |
| | | | | SD | 1.05 |
| | | | | CV % | 7.2 |
| | | | | Median | 14.70 |
| | | Day 70 | Sandostatin LAR ® 20 mg | n | 10 |
| | | | | Mean | 14.52 |
| | | | | SD | 1.20 |
| | | | | CV % | 8.3 |
| | | | | Median | 14.40 |
| Lymphocytes | E+9/L | Screening | Sandostatin LAR ® 20 mg | n | 10 |
| | | | | Mean | 2.04 |
| | | | | SD | 0.67 |
| | | | | CV % | 33.0 |
| | | | | Median | 1.86 |
| | | Day−1 | Sandostatin LAR ® 20 mg | n | 10 |
| | | | | Mean | 1.91 |
| | | | | SD | 0.65 |
| | | | | CV % | 34.0 |
| | | | | Median | 1.82 |
| | | Day 2 | Sandostatin LAR ® 20 mg | n | 10 |
| | | | | Mean | 2.20 |
| | | | | SD | 0.55 |
| | | | | CV % | 24.9 |
| | | | | Median | 2.05 |
| | | Day 28 | Sandostatin LAR ® 20 mg | n | 10 |
| | | | | Mean | 2.41 |
| | | | | SD | 0.82 |
| | | | | CV % | 34.1 |
| | | | | Median | 2.11 |
| | | Day 70 | Sandostatin LAR ® 20 mg | n | 10 |
| | | | | Mean | 2.34 |
| | | | | SD | 0.72 |
| | | | | CV % | 31.0 |
| | | | | Median | 2.12 |
| MCH | pg | Screening | Sandostatin LAR ® 20 mg | n | 10 |
| | | | | Mean | 29.98 |
| | | | | SD | 1.06 |
| | | | | CV % | 3.5 |
| | | | | Median | 29.85 |
| | | Day−1 | Sandostatin LAR ® 20 mg | n | 10 |
| | | | | Mean | 29.95 |
| | | | | SD | 0.93 |
| | | | | CV % | 3.1 |
| | | | | Median | 29.60 |
| | | Day 2 | Sandostatin LAR ® 20 mg | n | 10 |
| | | | | Mean | 30.22 |
| | | | | SD | 1.02 |
| | | | | CV % | 3.4 |
| | | | | Median | 29.85 |
| | | Day 28 | Sandostatin LAR ® 20 mg | n | 10 |
| | | | | Mean | 29.87 |
| | | | | SD | 0.93 |
| | | | | CV % | 3.1 |
| | | | | Median | 29.65 |
| | | Day 70 | Sandostatin LAR ® 20 mg | n | 10 |
| | | | | Mean | 29.82 |
| | | | | SD | 0.98 |
| | | | | CV % | 3.3 |
| | | | | Median | 29.60 |
| MCHC | g/dL | Screening | Sandostatin LAR ® 20 mg | n | 10 |
| | | | | Mean | 33.54 |
| | | | | SD | 0.81 |
| | | | | CV % | 2.4 |
| | | | | Median | 33.60 |
| | | Day−1 | Sandostatin LAR ® 20 mg | n | 10 |
| | | | | Mean | 33.61 |
| | | | | SD | 0.70 |
| | | | | CV % | 2.1 |
| | | | | Median | 33.65 |
| | | Day 2 | Sandostatin LAR ® 20 mg | n | 10 |
| | | | | Mean | 34.06 |
| | | | | SD | 0.51 |
| | | | | CV % | 1.5 |
| | | | | Median | 34.05 |
| | | Day 28 | Sandostatin LAR ® 20 mg | n | 10 |
| | | | | Mean | 33.32 |
| | | | | SD | 1.05 |
| | | | | CV % | 3.1 |
| | | | | Median | 33.50 |
| | | Day 70 | Sandostatin LAR ® 20 mg | n | 10 |
| | | | | Mean | 33.22 |
| | | | | SD | 0.85 |
| | | | | CV % | 2.6 |
| | | | | Median | 33.40 |
| Mean Cell Volume | fL | Screening | Sandostatin LAR ® 20 mg | n | 10 |
| | | | | Mean | 89.43 |
| | | | | SD | 3.27 |
| | | | | CV % | 3.7 |
| | | | | Median | 88.90 |
| | | Day−1 | Sandostatin LAR ® 20 mg | n | 10 |
| | | | | Mean | 89.20 |
| | | | | SD | 3.22 |
| | | | | CV % | 3.6 |
| | | | | Median | 89.55 |
| | | Day 2 | Sandostatin LAR ® 20 mg | n | 10 |
| | | | | Mean | 88.75 |
| | | | | SD | 3.41 |
| | | | | CV % | 3.8 |
| | | | | Median | 88.80 |
| | | Day 28 | Sandostatin LAR ® 20 mg | n | 10 |
| | | | | Mean | 89.77 |
| | | | | SD | 3.62 |
| | | | | CV % | 4.0 |
| | | | | Median | 90.10 |
| | | Day 70 | Sandostatin LAR ® 20 mg | n | 10 |
| | | | | Mean | 89.77 |
| | | | | SD | 2.79 |
| | | | | CV % | 3.1 |
| | | | | Median | 90.10 |
| Monocytes | E + 9/L | Screening | Sandostatin LAR ® 20 mg | n | 10 |
| | | | | Mean | 0.51 |
| | | | | SD | 0.15 |
| | | | | CV % | 28.9 |
| | | | | Median | 0.52 |
| | | Day−1 | Sandostatin LAR ® 20 mg | n | 10 |
| | | | | Mean | 0.42 |
| | | | | SD | 0.14 |
| | | | | CV % | 32.6 |
| | | | | Median | 0.43 |
| | | Day 2 | Sandostatin LAR ® 20 mg | n | 10 |
| | | | | Mean | 0.50 |
| | | | | SD | 0.14 |
| | | | | CV % | 27.4 |
| | | | | Median | 0.51 |
| | | Day 28 | Sandostatin LAR ® 20 mg | n | 10 |
| | | | | Mean | 0.51 |
| | | | | SD | 0.14 |
| | | | | CV % | 27.4 |
| | | | | Median | 0.49 |
| | | Day 70 | Sandostatin LAR ® 20 mg | n | 10 |
| | | | | Mean | 0.46 |
| | | | | SD | 0.11 |
| | | | | CV % | 23.0 |
| | | | | Median | 0.43 |
| Neutrophils | E + 9/L | Screening | Sandostatin LAR ® 20 mg | n | 10 |
| | | | | Mean | 3.86 |
| | | | | SD | 1.01 |
| | | | | CV % | 26.1 |
| | | | | Median | 4.00 |
| | | Day−1 | Sandostatin LAR ® 20 mg | n | 10 |
| | | | | Mean | 3.22 |
| | | | | SD | 1.16 |
| | | | | CV % | 36.1 |
| | | | | Median | 3.40 |
| | | Day 2 | Sandostatin LAR ® 20 mg | n | 10 |
| | | | | Mean | 3.44 |
| | | | | SD | 1.04 |
| | | | | CV % | 30.3 |
| | | | | Median | 3.37 |
| | | Day 28 | Sandostatin LAR ® 20 mg | n | 10 |
| | | | | Mean | 2.82 |
| | | | | SD | 0.90 |
| | | | | CV % | 31.8 |
| | | | | Median | 2.93 |
| | | Day 70 | Sandostatin LAR ® 20 mg | n | 10 |
| | | | | Mean | 2.75 |
| | | | | SD | 0.77 |
| | | | | CV % | 28.1 |
| | | | | Median | 2.86 |
| Packed Cell Volume | | Screening | Sandostatin LAR ® 20 mg | n | 10 |
| | | | | Mean | 0.46 |
| | | | | SD | 0.02 |
| | | | | CV % | 5.4 |
| | | | | Median | 0.46 |
| | | Day−1 | Sandostatin LAR ® 20 mg | n | 10 |
| | | | | Mean | 0.45 |
| | | | | SD | 0.03 |
| | | | | CV % | 6.3 |
| | | | | Median | 0.46 |
| | | Day 2 | Sandostatin LAR ® 20 mg | n | 10 |
| | | | | Mean | 0.48 |
| | | | | SD | 0.02 |
| | | | | CV % | 4.2 |
| | | | | Median | 0.48 |
| | | Day 28 | Sandostatin LAR ® 20 mg | n | 10 |
| | | | | Mean | 0.44 |
| | | | | SD | 0.03 |
| | | | | CV % | 6.1 |
| | | | | Median | 0.45 |
| | | Day 70 | Sandostatin LAR ® 20 mg | n | 10 |
| | | | | Mean | 0.44 |
| | | | | SD | 0.03 |
| | | | | CV % | 6.9 |
| | | | | Median | 0.43 |
| Platelet Count | E + 9/L | Screening | Sandostatin LAR ® 20 mg | n | 10 |
| | | | | Mean | 229.1 |
| | | | | SD | 66.53 |
| | | | | CV % | 29.0 |
| | | | | Median | 215.0 |
| | | Day−1 | Sandostatin LAR ® 20 mg | n | 10 |
| | | | | Mean | 237.1 |
| | | | | SD | 62.01 |
| | | | | CV % | 26.2 |
| | | | | Median | 227.5 |
| | | Day 2 | Sandostatin LAR ® 20 mg | n | 10 |
| | | | | Mean | 241.7 |
| | | | | SD | 55.02 |
| | | | | CV % | 22.8 |
| | | | | Median | 243.0 |
| | | Day 28 | Sandostatin LAR ® 20 mg | n | 10 |
| | | | | Mean | 223.8 |
| | | | | SD | 56.46 |
| | | | | CV % | 25.2 |
| | | | | Median | 221.0 |
| | | Day 70 | Sandostatin LAR ® 20 mg | n | 10 |
| | | | | Mean | 225.7 |
| | | | | SD | 63.23 |
| | | | | CV % | 28.0 |
| | | | | Median | 206.5 |
| Red Cell Count | E + 12/L | Screening | Sandostatin LAR ® 20 mg | n | 10 |
| | | | | Mean | 5.18 |
| | | | | SD | 0.40 |
| | | | | CV % | 7.8 |
| | | | | Median | 5.03 |
| | | Day−1 | Sandostatin LAR ® 20 mg | n | 10 |
| | | | | Mean | 5.11 |
| | | | | SD | 0.43 |
| | | | | CV % | 8.4 |
| | | | | Median | 5.13 |
| | | Day 2 | Sandostatin LAR ® 20 mg | n | 10 |
| | | | | Mean | 5.37 |
| | | | | SD | 0.37 |
| | | | | CV % | 7.0 |
| | | | | Median | 5.30 |
| | | Day 28 | Sandostatin LAR ® 20 mg | n | 10 |
| | | | | Mean | 4.93 |
| | | | | SD | 0.40 |
| | | | | CV % | 8.1 |
| | | | | Median | 4.95 |
| | | Day 70 | Sandostatin LAR ® 20 mg | n | 10 |
| | | | | Mean | 4.88 |
| | | | | SD | 0.45 |
| | | | | CV % | 9.3 |
| | | | | Median | 4.74 |
| White Cell Count | E + 9/L | Screening | Sandostatin LAR ® 20 mg | n | 10 |
| | | | | Mean | 6.62 |
| | | | | SD | 1.61 |
| | | | | CV % | 24.3 |
| | | | | Median | 6.90 |
| | | Day−1 | Sandostatin LAR ® 20 mg | n | 10 |
| | | | | Mean | 5.74 |
| | | | | SD | 1.76 |
| | | | | CV % | 30.7 |
| | | | | Median | 5.90 |
| | | Day 2 | Sandostatin LAR ® 20 mg | n | 10 |
| | | | | Mean | 6.37 |
| | | | | SD | 1.34 |
| | | | | CV % | 21.1 |
| | | | | Median | 6.45 |
| | | Day 28 | Sandostatin LAR ® 20 mg | n | 10 |
| | | | | Mean | 6.08 |
| | | | | SD | 1.57 |
| | | | | CV % | 25.9 |
| | | | | Median | 5.90 |
| | | Day 70 | Sandostatin LAR ® 20 mg | n | 10 |
| | | | | Mean | 5.80 |
| | | | | SD | 1.30 |
| | | | | CV % | 22.4 |
| | | | | Median | 5.50 |

TABLE 6-9
Summary of Biochemistry Parameters
Summary of Biochemistry Laboratory Values
Safety Population

| Laboratory Test | Units | Visit (Day) | Treatment | Summary Statistic | Value |
|---|---|---|---|---|---|
| ALT | IU/L | Screening | ATRIGEL ®/Octreotide 20 mg | n | 10 |
| | | | | Mean | 20.30 |
| | | | | SD | 5.46 |
| | | | | CV % | 26.9 |
| | | | | Median | 20.50 |
| | | Day−1 | ATRIGEL ®/Octreotide 20 mg | n | 10 |
| | | | | Mean | 20.40 |
| | | | | SD | 6.74 |
| | | | | CV % | 33.0 |
| | | | | Median | 18.50 |
| | | Day 2 | ATRIGEL ®/Octreotide 20 mg | n | 10 |
| | | | | Mean | 18.90 |
| | | | | SD | 5.92 |
| | | | | CV % | 31.3 |
| | | | | Median | 17.50 |
| | | Day 28 | ATRIGEL ®/Octreotide 20 mg | n | 10 |
| | | | | Mean | 18.90 |
| | | | | SD | 6.54 |
| | | | | CV % | 34.6 |
| | | | | Median | 18.00 |
| | | Day 70 | ATRIGEL ®/Octreotide 20 mg | n | 10 |
| | | | | Mean | 20.70 |
| | | | | SD | 9.29 |
| | | | | CV % | 44.9 |
| | | | | Median | 19.00 |
| AST | IU/L | Screening | ATRIGEL ®/Octreotide 20 mg | n | 10 |
| | | | | Mean | 21.30 |
| | | | | SD | 3.23 |
| | | | | CV % | 15.2 |
| | | | | Median | 21.00 |
| | | Day−1 | ATRIGEL ®/Octreotide 20 mg | n | 10 |
| | | | | Mean | 22.60 |
| | | | | SD | 5.25 |
| | | | | CV % | 23.2 |
| | | | | Median | 21.00 |
| | | Day 2 | ATRIGEL ®/Octreotide 20 mg | n | 10 |
| | | | | Mean | 22.60 |
| | | | | SD | 3.81 |
| | | | | CV % | 16.8 |
| | | | | Median | 21.00 |
| | | Day 28 | ATRIGEL ®/Octreotide 20 mg | n | 10 |
| | | | | Mean | 21.30 |
| | | | | SD | 4.08 |
| | | | | CV % | 19.2 |
| | | | | Median | 21.00 |
| | | Day 70 | ATRIGEL ®/Octreotide 20 mg | n | 10 |
| | | | | Mean | 25.60 |
| | | | | SD | 10.04 |
| | | | | CV % | 39.2 |
| | | | | Median | 21.50 |
| Albumin | g/L | Screening | ATRIGEL ®/Octreotide 20 mg | n | 10 |
| | | | | Mean | 43.70 |
| | | | | SD | 2.75 |
| | | | | CV % | 6.3 |
| | | | | Median | 43.50 |
| | | Day−1 | ATRIGEL ®/Octreotide 20 mg | n | 10 |
| | | | | Mean | 43.60 |
| | | | | SD | 2.12 |
| | | | | CV % | 4.9 |
| | | | | Median | 44.00 |
| | | Day 2 | ATRIGEL ®/Octreotide 20 mg | n | 10 |
| | | | | Mean | 44.60 |
| | | | | SD | 2.22 |
| | | | | CV % | 5.0 |
| | | | | Median | 44.50 |
| | | Day 28 | ATRIGEL ®/Octreotide 20 mg | n | 10 |
| | | | | Mean | 42.60 |
| | | | | SD | 1.90 |
| | | | | CV % | 4.5 |
| | | | | Median | 42.50 |
| | | Day 70 | ATRIGEL ®/Octreotide 20 mg | n | 10 |
| | | | | Mean | 41.30 |
| | | | | SD | 1.64 |
| | | | | CV % | 4.0 |
| | | | | Median | 41.00 |
| Alkaline Phosphatase | IU/L | Screening | ATRIGEL ®/Octreotide 20 mg | n | 10 |
| | | | | Mean | 62.60 |
| | | | | SD | 17.56 |
| | | | | CV % | 28.1 |
| | | | | Median | 67.00 |
| | | Day−1 | ATRIGEL ®/Octreotide 20 mg | n | 10 |
| | | | | Mean | 57.70 |
| | | | | SD | 14.47 |
| | | | | CV % | 25.1 |
| | | | | Median | 56.00 |
| | | Day 2 | ATRIGEL ®/Octreotide 20 mg | n | 10 |
| | | | | Mean | 61.00 |
| | | | | SD | 17.01 |
| | | | | CV % | 27.9 |
| | | | | Median | 59.50 |
| | | Day 28 | ATRIGEL ®/Octreotide 20 mg | n | 10 |
| | | | | Mean | 57.60 |
| | | | | SD | 14.66 |
| | | | | CV % | 25.5 |
| | | | | Median | 59.50 |
| | | Day 70 | ATRIGEL ®/Octreotide 20 mg | n | 10 |
| | | | | Mean | 58.70 |
| | | | | SD | 15.97 |
| | | | | CV % | 27.2 |
| | | | | Median | 62.00 |
| Bicarbonate | mmol/L | Screening | ATRIGEL ®/Octreotide 20 mg | n | 10 |
| | | | | Mean | 27.80 |
| | | | | SD | 0.92 |
| | | | | CV % | 3.3 |
| | | | | Median | 28.00 |
| | | Day−1 | ATRIGEL ®/Octreotide 20 mg | n | 10 |
| | | | | Mean | 27.90 |
| | | | | SD | 0.74 |
| | | | | CV % | 2.6 |
| | | | | Median | 28.00 |
| | | Day 2 | ATRIGEL ®/Octreotide 20 mg | n | 10 |
| | | | | Mean | 28.30 |
| | | | | SD | 2.50 |
| | | | | CV % | 8.8 |
| | | | | Median | 27.50 |
| | | Day 28 | ATRIGEL ®/Octreotide 20 mg | n | 10 |
| | | | | Mean | 27.80 |
| | | | | SD | 1.14 |
| | | | | CV % | 4.1 |
| | | | | Median | 28.00 |
| | | Day 70 | ATRIGEL ®/Octreotide 20 mg | n | 10 |
| | | | | Mean | 26.90 |
| | | | | SD | 1.20 |
| | | | | CV % | 4.5 |
| | | | | Median | 27.00 |
| Bilirubin | μmol/L | Screening | ATRIGEL ®/Octreotide 20 mg | n | 10 |
| | | | | Mean | 13.00 |
| | | | | SD | 7.33 |
| | | | | CV % | 56.4 |
| | | | | Median | 11.50 |
| | | Day−1 | ATRIGEL ®/Octreotide 20 mg | n | 10 |
| | | | | Mean | 16.70 |
| | | | | SD | 7.06 |
| | | | | CV % | 42.3 |
| | | | | Median | 15.00 |
| | | Day 2 | ATRIGEL ®/Octreotide 20 mg | n | 10 |
| | | | | Mean | 27.20 |
| | | | | SD | 17.05 |
| | | | | CV % | 62.7 |
| | | | | Median | 22.00 |
| | | Day 28 | ATRIGEL ®/Octreotide 20 mg | n | 10 |
| | | | | Mean | 14.80 |
| | | | | SD | 7.71 |
| | | | | CV % | 52.1 |
| | | | | Median | 11.50 |
| | | Day 70 | ATRIGEL ®/Octreotide 20 mg | n | 10 |
| | | | | Mean | 14.10 |
| | | | | SD | 7.59 |
| | | | | CV % | 53.9 |
| | | | | Median | 13.50 |
| Calcium | mmol/L | Screening | ATRIGEL ®/Octreotide 20 mg | n | 10 |
| | | | | Mean | 2.36 |
| | | | | SD | 0.08 |
| | | | | CV % | 3.5 |
| | | | | Median | 2.36 |
| | | Day−1 | ATRIGEL ®/Octreotide 20 mg | n | 10 |
| | | | | Mean | 2.31 |
| | | | | SD | 0.08 |
| | | | | CV % | 3.4 |
| | | | | Median | 2.30 |
| | | Day 2 | ATRIGEL ®/Octreotide 20 mg | n | 10 |
| | | | | Mean | 2.32 |
| | | | | SD | 0.06 |
| | | | | CV % | 2.7 |
| | | | | Median | 2.29 |
| | | Day 28 | ATRIGEL ®/Octreotide 20 mg | n | 10 |
| | | | | Mean | 2.30 |
| | | | | SD | 0.06 |
| | | | | CV % | 2.4 |
| | | | | Median | 2.31 |
| | | Day 70 | ATRIGEL ®/Octreotide 20 mg | n | 10 |
| | | | | Mean | 2.28 |
| | | | | SD | 0.08 |
| | | | | CV % | 3.3 |
| | | | | Median | 2.27 |
| Chloride | mmol/L | Screening | ATRIGEL ®/Octreotide 20 mg | n | 10 |
| | | | | Mean | 106.0 |
| | | | | SD | 1.05 |
| | | | | CV % | 1.0 |
| | | | | Median | 106.0 |
| | | Day−1 | ATRIGEL ®/Octreotide 20 mg | n | 10 |
| | | | | Mean | 104.9 |
| | | | | SD | 1.29 |
| | | | | CV % | 1.2 |
| | | | | Median | 105.0 |
| | | Day 2 | ATRIGEL ®/Octreotide 20 mg | n | 10 |
| | | | | Mean | 103.1 |
| | | | | SD | 1.37 |
| | | | | CV % | 1.3 |
| | | | | Median | 103.0 |
| | | Day 28 | ATRIGEL ®/Octreotide 20 mg | n | 10 |
| | | | | Mean | 105.3 |
| | | | | SD | 1.25 |
| | | | | CV % | 1.2 |
| | | | | Median | 106.0 |
| | | Day 70 | ATRIGEL ®/Octreotide 20 mg | n | 10 |
| | | | | Mean | 105.7 |
| | | | | SD | 0.95 |
| | | | | CV % | 0.9 |
| | | | | Median | 106.0 |
| Cholesterol | mmol/L | Screening | ATRIGEL ®/Octreotide 20 mg | n | 10 |
| | | | | Mean | 4.70 |
| | | | | SD | 1.38 |
| | | | | CV % | 29.4 |
| | | | | Median | 4.35 |
| | | Day−1 | ATRIGEL ®/Octreotide 20 mg | n | 10 |
| | | | | Mean | 4.46 |
| | | | | SD | 1.16 |
| | | | | CV % | 25.9 |
| | | | | Median | 4.25 |
| | | Day 2 | ATRIGEL ®/Octreotide 20 mg | n | 10 |
| | | | | Mean | 5.13 |
| | | | | SD | 1.38 |
| | | | | CV % | 26.9 |
| | | | | Median | 4.60 |
| | | Day 28 | ATRIGEL ®/Octreotide 20 mg | n | 10 |
| | | | | Mean | 4.65 |
| | | | | SD | 1.37 |
| | | | | CV % | 29.4 |
| | | | | Median | 4.65 |
| | | Day 70 | ATRIGEL ®/Octreotide 20 mg | n | 10 |
| | | | | Mean | 3.98 |
| | | | | SD | 0.81 |
| | | | | CV % | 20.5 |
| | | | | Median | 3.75 |
| Creatinine | μmol/L | Screening | ATRIGEL ®/Octreotide 20 mg | n | 10 |
| | | | | Mean | 90.40 |
| | | | | SD | 5.76 |
| | | | | CV % | 6.4 |
| | | | | Median | 89.50 |
| | | Day−1 | ATRIGEL ®/Octreotide 20 mg | n | 10 |
| | | | | Mean | 92.50 |
| | | | | SD | 7.62 |
| | | | | CV % | 8.2 |
| | | | | Median | 91.00 |
| | | Day 2 | ATRIGEL ®/Octreotide 20 mg | n | 10 |
| | | | | Mean | 92.00 |
| | | | | SD | 7.42 |
| | | | | CV % | 8.1 |
| | | | | Median | 93.00 |
| | | Day 28 | ATRIGEL ®/Octreotide 20 mg | n | 10 |
| | | | | Mean | 88.50 |
| | | | | SD | 8.41 |
| | | | | CV % | 9.5 |
| | | | | Median | 88.00 |
| | | Day 70 | ATRIGEL ®/Octreotide 20 mg | n | 10 |
| | | | | Mean | 86.20 |
| | | | | SD | 7.94 |
| | | | | CV % | 9.2 |
| | | | | Median | 84.00 |
| Gamma GT | IU/L | Screening | ATRIGEL ®/Octreotide 20 mg | n | 10 |
| | | | | Mean | 21.80 |
| | | | | SD | 10.38 |
| | | | | CV % | 47.6 |
| | | | | Median | 20.00 |
| | | Day−1 | ATRIGEL ®/Octreotide 20 mg | n | 10 |
| | | | | Mean | 20.00 |
| | | | | SD | 11.60 |
| | | | | CV % | 58.0 |
| | | | | Median | 16.50 |
| | | Day 2 | ATRIGEL ®/Octreotide 20 mg | n | 10 |
| | | | | Mean | 20.00 |
| | | | | SD | 11.86 |
| | | | | CV % | 59.3 |
| | | | | Median | 16.00 |
| | | Day 28 | ATRIGEL ®/Octreotide 20 mg | n | 10 |
| | | | | Mean | 25.70 |
| | | | | SD | 19.91 |
| | | | | CV % | 77.5 |
| | | | | Median | 17.50 |
| | | Day 70 | ATRIGEL ®/Octreotide 20 mg | n | 10 |
| | | | | Mean | 22.20 |
| | | | | SD | 15.85 |
| | | | | CV % | 71.4 |
| | | | | Median | 15.00 |
| Globulin | g/L | Screening | ATRIGEL ®/Octreotide 20 mg | n | 10 |
| | | | | Mean | 29.80 |
| | | | | SD | 2.94 |
| | | | | CV % | 9.9 |
| | | | | Median | 30.00 |
| | | Day−1 | ATRIGEL ®/Octreotide 20 mg | n | 10 |
| | | | | Mean | 27.90 |
| | | | | SD | 2.64 |
| | | | | CV % | 9.5 |
| | | | | Median | 28.00 |
| | | Day 2 | ATRIGEL ®/Octreotide 20 mg | n | 10 |
| | | | | Mean | 29.20 |
| | | | | SD | 1.55 |
| | | | | CV % | 5.3 |
| | | | | Median | 29.50 |
| | | Day 28 | ATRIGEL ®/Octreotide 20 mg | n | 10 |
| | | | | Mean | 26.20 |
| | | | | SD | 2.39 |
| | | | | CV % | 9.1 |
| | | | | Median | 26.00 |
| | | Day 70 | ATRIGEL ®/Octreotide 20 mg | n | 10 |
| | | | | Mean | 26.90 |
| | | | | SD | 2.08 |
| | | | | CV % | 7.7 |
| | | | | Median | 28.00 |
| Glucose | mmol/L | Screening | ATRIGEL ®/Octreotide 20 mg | n | 10 |
| | | | | Mean | 5.09 |
| | | | | SD | 0.58 |
| | | | | CV % | 11.4 |
| | | | | Median | 4.90 |
| | | Day−1 | ATRIGEL ®/Octreotide 20 mg | n | 10 |
| | | | | Mean | 5.13 |
| | | | | SD | 0.54 |
| | | | | CV % | 10.5 |
| | | | | Median | 5.15 |
| | | Day 2 | ATRIGEL ®/Octreotide 20 mg | n | 10 |
| | | | | Mean | 5.23 |
| | | | | SD | 0.55 |
| | | | | CV % | 10.6 |
| | | | | Median | 5.15 |
| | | Day 28 | ATRIGEL ®/Octreotide 20 mg | n | 10 |
| | | | | Mean | 5.42 |
| | | | | SD | 0.58 |
| | | | | CV % | 10.7 |
| | | | | Median | 5.40 |
| | | Day 70 | ATRIGEL ®/Octreotide 20 mg | n | 10 |
| | | | | Mean | 5.05 |
| | | | | SD | 0.43 |
| | | | | CV % | 8.5 |
| | | | | Median | 4.95 |
| Inorganic Phosphorus | mmol/L | Screening | ATRIGEL ®/Octreotide 20 mg | n | 10 |
| | | | | Mean | 1.21 |
| | | | | SD | 0.13 |
| | | | | CV % | 10.8 |
| | | | | Median | 1.19 |
| | | Day−1 | ATRIGEL ®/Octreotide 20 mg | n | 10 |
| | | | | Mean | 0.97 |
| | | | | SD | 0.13 |
| | | | | CV % | 13.3 |
| | | | | Median | 0.96 |
| | | Day 2 | ATRIGEL ®/Octreotide 20 mg | n | 10 |
| | | | | Mean | 1.00 |
| | | | | SD | 0.10 |
| | | | | CV % | 10.4 |
| | | | | Median | 1.02 |
| | | Day 28 | ATRIGEL ®/Octreotide 20 mg | n | 10 |
| | | | | Mean | 1.15 |
| | | | | SD | 0.16 |
| | | | | CV % | 13.6 |
| | | | | Median | 1.16 |
| | | Day 70 | ATRIGEL ®/Octreotide 20 mg | n | 10 |
| | | | | Mean | 1.14 |
| | | | | SD | 0.16 |
| | | | | CV % | 14.2 |
| | | | | Median | 1.12 |
| Potassium | mmol/L | Screening | ATRIGEL ®/Octreotide 20 mg | n | 10 |
| | | | | Mean | 4.43 |
| | | | | SD | 0.22 |
| | | | | CV % | 5.0 |
| | | | | Median | 4.45 |
| | | Day−1 | ATRIGEL ®/Octreotide 20 mg | n | 10 |
| | | | | Mean | 4.37 |
| | | | | SD | 0.51 |
| | | | | CV % | 11.7 |
| | | | | Median | 4.35 |
| | | Day 2 | ATRIGEL ®/Octreotide 20 mg | n | 10 |
| | | | | Mean | 4.65 |
| | | | | SD | 0.52 |
| | | | | CV % | 11.2 |
| | | | | Median | 4.60 |
| | | Day 28 | ATRIGEL ®/Octreotide 20 mg | n | 10 |
| | | | | Mean | 4.36 |
| | | | | SD | 0.24 |
| | | | | CV % | 5.5 |
| | | | | Median | 4.40 |
| | | Day 70 | ATRIGEL ®/Octreotide 20 mg | n | 10 |
| | | | | Mean | 4.38 |
| | | | | SD | 0.40 |
| | | | | CV % | 9.2 |
| | | | | Median | 4.25 |
| Sodium | mmol/L | Screening | ATRIGEL ®/Octreotide 20 mg | n | 10 |
| | | | | Mean | 140.5 |
| | | | | SD | 0.97 |
| | | | | CV % | 0.7 |
| | | | | Median | 141.0 |
| | | Day−1 | ATRIGEL ®/Octreotide 20 mg | n | 10 |
| | | | | Mean | 138.4 |
| | | | | SD | 1.26 |
| | | | | CV % | 0.9 |
| | | | | Median | 138.0 |
| | | Day 2 | ATRIGEL ®/Octreotide 20 mg | n | 10 |
| | | | | Mean | 139.1 |
| | | | | SD | 2.51 |
| | | | | CV % | 1.8 |
| | | | | Median | 138.5 |
| | | Day 28 | ATRIGEL ®/Octreotide 20 mg | n | 10 |
| | | | | Mean | 139.6 |
| | | | | SD | 1.17 |
| | | | | CV % | 0.8 |
| | | | | Median | 139.0 |
| | | Day 70 | ATRIGEL ®/Octreotide 20 mg | n | 10 |
| | | | | Mean | 138.8 |
| | | | | SD | 1.40 |
| | | | | CV % | 1.0 |
| | | | | Median | 139.0 |
| Total Protein | g/L | Screening | ATRIGEL ®/Octreotide 20 mg | n | 10 |
| | | | | Mean | 73.50 |
| | | | | SD | 3.81 |
| | | | | CV % | 5.2 |
| | | | | Median | 74.50 |
| | | Day−1 | ATRIGEL ®/Octreotide 20 mg | n | 10 |
| | | | | Mean | 71.50 |
| | | | | SD | 3.54 |
| | | | | CV % | 4.9 |
| | | | | Median | 72.50 |
| | | Day 2 | ATRIGEL ®/Octreotide 20 mg | n | 10 |
| | | | | Mean | 73.80 |
| | | | | SD | 2.66 |
| | | | | CV % | 3.6 |
| | | | | Median | 73.50 |
| | | Day 28 | ATRIGEL ®/Octreotide 20 mg | n | 10 |
| | | | | Mean | 68.80 |
| | | | | SD | 2.94 |
| | | | | CV % | 4.3 |
| | | | | Median | 68.00 |
| | | Day 70 | ATRIGEL ®/Octreotide 20 mg | n | 10 |
| | | | | Mean | 68.20 |
| | | | | SD | 3.29 |
| | | | | CV % | 4.8 |
| | | | | Median | 67.50 |
| Urea | mmol/L | Screening | ATRIGEL ®/Octreotide 20 mg | n | 10 |
| | | | | Mean | 5.43 |
| | | | | SD | 1.54 |
| | | | | CV % | 28.3 |
| | | | | Median | 5.15 |
| | | Day−1 | ATRIGEL ®/Octreotide 20 mg | n | 10 |
| | | | | Mean | 5.47 |
| | | | | SD | 1.16 |
| | | | | CV % | 21.2 |
| | | | | Median | 5.45 |
| | | Day 2 | ATRIGEL ®/Octreotide 20 mg | n | 10 |
| | | | | Mean | 5.03 |
| | | | | SD | 0.90 |
| | | | | CV % | 17.8 |
| | | | | Median | 5.05 |
| | | Day 28 | ATRIGEL ®/Octreotide 20 mg | n | 10 |
| | | | | Mean | 5.67 |
| | | | | SD | 1.05 |
| | | | | CV % | 18.6 |
| | | | | Median | 5.90 |
| | | Day 70 | ATRIGEL ®/Octreotide 20 mg | n | 10 |
| | | | | Mean | 4.97 |
| | | | | SD | 0.70 |
| | | | | CV % | 14.0 |
| | | | | Median | 4.65 |
| Uric Acid | μmol/L | Screening | ATRIGEL ®/Octreotide 20 mg | n | 10 |
| | | | | Mean | 334.9 |
| | | | | SD | 56.54 |
| | | | | CV % | 16.9 |
| | | | | Median | 350.0 |
| | | Day−1 | ATRIGEL ®/Octreotide 20 mg | n | 10 |
| | | | | Mean | 358.5 |
| | | | | SD | 65.34 |
| | | | | CV % | 18.2 |
| | | | | Median | 343.0 |
| | | Day 2 | ATRIGEL ®/Octreotide 20 mg | n | 10 |
| | | | | Mean | 422.9 |
| | | | | SD | 80.20 |
| | | | | CV % | 19.0 |
| | | | | Median | 407.5 |
| | | Day 28 | ATRIGEL ®/Octreotide 20 mg | n | 10 |
| | | | | Mean | 367.6 |
| | | | | SD | 65.25 |
| | | | | CV % | 17.7 |
| | | | | Median | 376.0 |
| | | Day 70 | ATRIGEL ®/Octreotide 20 mg | n | 10 |
| | | | | Mean | 349.7 |
| | | | | SD | 71.72 |
| | | | | CV % | 20.5 |
| | | | | Median | 352.0 |

Injection Site Assessment Summary

TABLE 6-10.1
Injection Site Assessment Summary

| | | | Erythema | | | | |
|---|---|---|---|---|---|---|---|
| Treatment | Day | Nominal Time | Absent | | Mild | Moderate | Severe |
| ATRIGEL ®/ | 1 | Pre-dose | 10 | (100%) | 0 (0%) | 0 (0%) | 0 (0%) |
| Octreotide 20 mg | | 0.25 h | 0 | (0%) | 0 (0%) | 9 (90%) | 1 (10%) |
| | | 0.50 h | 0 | (0%) | 0 (0%) | 8 (80%) | 2 (20%) |
| | | 1 h | 0 | (0%) | 3 (30%) | 7 (70%) | 0 (0%) |
| | | 2 h | 2 | (20%) | 8 (80%) | 0 (0%) | 0 (0%) |
| | | 3 h | 2 | (20%) | 8 (80%) | 0 (0%) | 0 (0%) |
| | | 4 h | 2 | (20%) | 8 (80%) | 0 (0%) | 0 (0%) |
| | | 8 h | 9 | (90%) | 1 (10%) | 0 (0%) | 0 (0%) |
| | | 12 h | 9 | (90%) | 1 (10%) | 0 (0%) | 0 (0%) |
| | | 16 h | 9 | (90%) | 1 (10%) | 0 (0%) | 0 (0%) |
| | 2 | 24 h | 9 | (90%) | 1 (10%) | 0 (0%) | 0 (0%) |
| | 3 | NA | 6 | (60%) | 3 (30%) | 1 (10%) | 0 (0%) |
| | 7 | NA | 9 | (90%) | 0 (0%) | 1 (10%) | 0 (0%) |
| | 14 | NA | 7 | (70%) | 0 (0%) | 1 (10%) | 2 (20%) |
| | 21 | NA | 8 | (80%) | 1 (10%) | 1 (10%) | 0 (0%) |
| | 28 | NA | 8 | (80%) | 2 (20%) | 0 (0%) | 0 (0%) |
| | 35 | NA | 10 | (100%) | 0 (0%) | 0 (0%) | 0 (0%) |
| | 42 | NA | 10 | (100%) | 0 (0%) | 0 (0%) | 0 (0%) |
| | 49 | NA | 10 | (100%) | 0 (0%) | 0 (0%) | 0 (0%) |
| | 56 | NA | 10 | (100%) | 0 (0%) | 0 (0%) | 0 (0%) |
| | 70 | NA | 10 | (100%) | 0 (0%) | 0 (0%) | 0 (0%) |

TABLE 6-10.2
Injection Site Assessment Summary

| | | | Erythema | | | | |
|---|---|---|---|---|---|---|---|
| Treatment | Day | Nominal Time | Absent | | Mild | Moderate | Severe |
| Sandostatin LAR ® 20 mg | 1 | Pre-dose | 10 | (100%) | 0 (0%) | 0 (0%) | 0 (0%) |
| | | 0.25 h | 8 | (80%) | 2 (20%) | 0 (0%) | 0 (0%) |
| | | 0.50 h | 8 | (80%) | 1 (10%) | 1 (10%) | 0 (0%) |
| | | 1 h | 10 | (100%) | 0 (0%) | 0 (0%) | 0 (0%) |
| | | 2 h | 10 | (100%) | 0 (0%) | 0 (0%) | 0 (0%) |
| | | 3 h | 10 | (100%) | 0 (0%) | 0 (0%) | 0 (0%) |
| | | 4 h | 10 | (100%) | 0 (0%) | 0 (0%) | 0 (0%) |
| | | 8 h | 9 | (90%) | 1 (10%) | 0 (0%) | 0 (0%) |
| | | 12 h | 9 | (90%) | 1 (10%) | 0 (0%) | 0 (0%) |
| | | 16 h | 9 | (90%) | 1 (10%) | 0 (0%) | 0 (0%) |
| | 2 | 24 h | 8 | (89%) | 1 (11%) | 0 (0%) | 0 (0%) |
| | 3 | NA | 7 | (70%) | 3 (30%) | 0 (0%) | 0 (0%) |
| | 7 | NA | 10 | (100%) | 0 (0%) | 0 (0%) | 0 (0%) |
| | 14 | NA | 10 | (100%) | 0 (0%) | 0 (0%) | 0 (0%) |
| | 21 | NA | 9 | (100%) | 0 (0%) | 0 (0%) | 0 (0%) |
| | 28 | NA | 10 | (100%) | 0 (0%) | 0 (0%) | 0 (0%) |
| | 35 | NA | 10 | (100%) | 0 (0%) | 0 (0%) | 0 (0%) |
| | 42 | NA | 9 | (90%) | 1 (10%) | 0 (0%) | 0 (0%) |
| | 49 | NA | 10 | (100%) | 0 (0%) | 0 (0%) | 0 (0%) |
| | 56 | NA | 10 | (100%) | 0 (0%) | 0 (0%) | 0 (0%) |
| | 70 | NA | 10 | (100%) | 0 (0%) | 0 (0%) | 0 (0%) |

Subjects 14 and 21 did not have assessments at day 2 and 21, respectively.

TABLE 6-11
TSH (mU/L) Summary Statistics
TSH (mU/L) Summary Statistics
Safety Population

| Treatment | Visit (Day) | Nominal time | n | Mean | SD | CV(%) | Median | Min | Max |
|---|---|---|---|---|---|---|---|---|---|
| ATRIGEL ®/Octreotide 20 mg | Day 1 | Pre-dose | 10 | 1.40 | 0.75 | 53.2 | 1.50 | 0.5 | 2.5 |
| | Day 2 | 24 h | 10 | 0.37 | 0.20 | 54.1 | 0.40 | 0.1 | 0.8 |
| | Day 3 | NA | 10 | 0.62 | 0.35 | 55.8 | 0.60 | 0.2 | 1.4 |
| | Day 7 | NA | 10 | 1.55 | 0.61 | 39.4 | 1.50 | 0.8 | 2.6 |
| | Day 14 | NA | 10 | 2.32 | 1.40 | 60.5 | 2.00 | 0.6 | 5.3 |
| | Day 21 | NA | 10 | 2.22 | 1.40 | 62.9 | 1.90 | 0.6 | 5.1 |
| | Day 28 | NA | 10 | 2.33 | 1.63 | 70.0 | 2.15 | 0.6 | 6.5 |
| | Day 70 | NA | 10 | 1.96 | 0.90 | 46.2 | 2.10 | 0.8 | 3.2 |
| Sandostatin LAR ® 20 mg | Day 1 | Pre-dose | 10 | 1.12 | 0.42 | 37.1 | 1.05 | 0.6 | 1.8 |
| | Day 2 | 24 h | 10 | 0.94 | 0.42 | 45.2 | 0.85 | 0.4 | 1.7 |
| | Day 3 | NA | 10 | 0.89 | 0.36 | 40.1 | 0.80 | 0.4 | 1.6 |
| | Day 7 | NA | 10 | 1.45 | 0.65 | 44.8 | 1.40 | 0.6 | 2.9 |
| | Day 14 | NA | 10 | 1.30 | 0.56 | 43.2 | 1.10 | 0.6 | 2.4 |
| | Day 21 | NA | 10 | 1.74 | 0.87 | 50.0 | 1.50 | 1.0 | 3.9 |
| | Day 28 | NA | 10 | 1.68 | 0.59 | 35.2 | 1.60 | 0.7 | 2.5 |
| | Day 70 | NA | 10 | 2.21 | 1.16 | 52.5 | 1.80 | 1.2 | 4.6 |

TABLE 6-12
Total T4 (μg/L) Summary Statistics
Total T4 (μg/L) Summary Statistics
Safety Population

| Treatment | Visit (Day) | Nominal time | n | Mean | SD | CV(%) | Median | Min | Max |
|---|---|---|---|---|---|---|---|---|---|
| ATRIGEL ®/Octreotide 20 mg | Day 1 | Pre-dose | 10 | 65.33 | 9.77 | 15.0 | 66.65 | 50.4 | 80.6 |
| | Day 2 | 24 h | 10 | 62.01 | 11.10 | 17.9 | 59.15 | 43.5 | 80.2 |
| | Day 3 | NA | 10 | 54.68 | 11.74 | 21.5 | 56.85 | 38.6 | 73.1 |
| | Day 7 | NA | 10 | 58.21 | 9.89 | 17.0 | 58.10 | 43.4 | 73.9 |
| | Day 14 | NA | 10 | 58.46 | 8.22 | 14.1 | 58.95 | 45.7 | 74.3 |
| | Day 21 | NA | 10 | 61.48 | 9.46 | 15.4 | 63.80 | 43.0 | 72.0 |
| | Day 28 | NA | 10 | 59.96 | 11.10 | 18.5 | 61.80 | 39.5 | 75.8 |
| | Day 70 | NA | 10 | 69.49 | 12.58 | 18.1 | 66.90 | 50.7 | 87.9 |
| Sandostatin LAR ® 20 mg | Day 1 | Pre-dose | 10 | 70.56 | 7.69 | 10.9 | 71.85 | 53.2 | 81.0 |
| | Day 2 | 24 h | 10 | 72.63 | 8.11 | 11.2 | 74.25 | 54.6 | 84.0 |
| | Day 3 | NA | 10 | 67.22 | 8.34 | 12.4 | 69.75 | 46.7 | 74.8 |
| | Day 7 | NA | 10 | 68.06 | 11.84 | 17.4 | 71.55 | 52.9 | 84.7 |
| | Day 14 | NA | 10 | 56.27 | 11.24 | 20.0 | 57.45 | 35.3 | 70.5 |
| | Day 21 | NA | 10 | 62.61 | 7.68 | 12.3 | 63.50 | 46.8 | 72.8 |
| | Day 28 | NA | 10 | 61.54 | 7.78 | 12.6 | 60.70 | 51.8 | 76.2 |
| | Day 70 | NA | 10 | 64.70 | 16.64 | 25.7 | 70.55 | 21.5 | 77.5 |

TABLE 6-13
Free T4 (pmol/L) Summary Statistics
Free T4 (pmol/L) Summary Statistics
Safety Population

| Treatment | Visit (Day) | Nominal time | n | Mean | SD | CV(%) | Median | Min | Max |
|---|---|---|---|---|---|---|---|---|---|
| ATRIGEL ®/Octreotide 20 mg | Day 1 | Pre-dose | 10 | 13.06 | 1.59 | 12.2 | 13.00 | 10.4 | 15.7 |
| | Day 2 | 24 h | 10 | 11.32 | 1.24 | 11.0 | 11.00 | 9.5 | 13.5 |
| | Day 3 | NA | 10 | 11.16 | 1.40 | 12.6 | 11.15 | 8.5 | 13.6 |
| | Day 7 | NA | 10 | 11.05 | 1.11 | 10.0 | 11.30 | 9.5 | 12.6 |
| | Day 14 | NA | 10 | 12.44 | 1.29 | 10.4 | 12.55 | 10.6 | 15.3 |
| | Day 21 | NA | 10 | 12.55 | 0.83 | 6.6 | 12.85 | 11.3 | 13.5 |
| | Day 28 | NA | 10 | 12.81 | 1.79 | 14.0 | 12.65 | 10.4 | 15.3 |
| | Day 70 | NA | 10 | 13.94 | 1.37 | 9.8 | 13.70 | 12.2 | 15.8 |
| Sandostatin LAR ® 20 mg | Day 1 | pre-dose | 10 | 13.00 | 1.15 | 8.9 | 13.10 | 11.1 | 14.5 |
| | Day 2 | 24 h | 10 | 11.80 | 1.57 | 13.3 | 11.65 | 9.9 | 14.3 |
| | Day 3 | NA | 10 | 12.09 | 1.13 | 9.3 | 12.10 | 10.3 | 14.2 |
| | Day 7 | NA | 10 | 11.95 | 2.00 | 16.7 | 12.00 | 9.3 | 14.8 |
| | Day 14 | NA | 10 | 10.97 | 1.26 | 11.5 | 10.90 | 8.9 | 12.7 |
| | Day 21 | NA | 10 | 11.86 | 1.00 | 8.5 | 11.70 | 10.4 | 13.5 |
| | Day 28 | NA | 10 | 12.02 | 1.49 | 12.4 | 11.50 | 10.3 | 15.1 |
| | Day 70 | NA | 10 | 13.12 | 0.77 | 5.9 | 13.35 | 11.7 | 14.2 |

TABLE 6-14.1
Individual and Summary Octreotide Pharmacokinetic Parameters
Following Administration of a Single s.c. Dose of
ATRIGEL ®/Octreotide 20 mg

| | | Cₘₐₓ | | AUC₀₋₂₄ | AUC₀₋ₜ |
|---|---|---|---|---|---|
| Subject | t_{Lag} (h) | (ng/mL) | tₘₐₓ (h) | (ng · h/mL) | (ng · h/mL) |
| 1 | 0.00 | 23.50 | 2.00 | 244 | 1312 |
| 4 | 0.00 | 84.60 | 3.00 | 661 | 934 |
| 6 | 0.00 | 22.60 | 3.00 | 261 | 940 |
| 11 | 0.00 | 32.50 | 2.02 | 279 | 3995 |
| 12 | 0.00 | 39.70 | 2.00 | 418 | 1237 |
| 13 | 0.00 | 35.30 | 2.00 | 277 | 1421 |
| 15 | 0.00 | 34.70 | 3.00 | 373 | 763 |
| 17 | 0.00 | 19.90 | 4.00 | 237 | 801 |
| 18 | 0.00 | 68.00 | 5.00 | 523 | 1104 |
| 20 | 0.00 | 22.90 | 4.00 | 259 | 1009 |
| N | 10 | 10 | 10 | 10 | 10 |
| Arithmetic | | 38.370 | | 353.2 | 1351.6 |
| mean | | 21.408 | | 142.4 | 953.3 |
| SD | | | | | |
| CV (%) | | 55.8 | | 40.3 | 70.5 |
| Median | 0.000 | 33.600 | 3.000 | 278.2 | 1056.7 |
| Min | 0.00 | 19.90 | 2.00 | 237 | 763 |
| Max | 0.00 | 84.6 | 5.00 | 661 | 3995 |
| Lower 95% CI | | 23.056 | | 251.3 | 669.6 |
| Upper 95% CI | | 53.684 | | 455.1 | 2033.5 |

TABLE 6-14.2
Individual and Summary Octreotide Pharmacokinetic Parameters
Following Administration of a Single i.m. Dose of
Sandostatin LAR ® 20 mg

| | | Cₘₐₓ | | AUC₀₋₂₄ | AUC₀₋ₜ |
|---|---|---|---|---|---|
| Subject | t_{Lag} (h) | (ng/mL) | tₘₐₓ (h) | (ng · h/mL) | (ng · h/mL) |
| 2 | 144.00 | 0.67 | 312.00 | 0 | 215 |
| 3 | 0.00 | 0.71 | 648.17 | 2 | 377 |
| 5 | 0.00 | 0.71 | 312.00 | 1 | 305 |
| 7 | 144.00 | 0.26 | 312.00 | 0 | 22 |
| 9 | 0.00 | 0.65 | 312.02 | 0 | 271 |
| 10 | 5.05 | 13.60 | 6.00 | 13 | 210 |
| 14 | 0.00 | 0.74 | 48.02 | 1 | 400 |
| 16 | 0.00 | 0.46 | 312.00 | 0 | 235 |
| 19 | 1.00 | 0.59 | 312.00 | 0 | 256 |
| 108 | 48.00 | 0.47 | 312.02 | 0 | 247 |
| N | 10 | 10 | 10 | 10 | 10 |
| Arithmetic | | 1.884 | | 1.8 | 253.7 |
| mean | | 4.119 | | 4.1 | 104.0 |
| SD | | | | | |
| CV (%) | | 218.6 | | 226.9 | 41.0 |
| Median | 0.500 | 0.660 | 312.000 | 0.3 | 251.4 |
| Min | 0.00 | 0.26 | 6.00 | 0 | 22 |
| Max | 144.00 | 13.60 | 648.17 | 13 | 400 |
| Lower 95% CI | | −1.063 | | −1.1 | 179.3 |
| Upper 95% CI | | 4.831 | | 4.7 | 328.2 |

### Example 7

### Evaluation of Octreotide/ATRIGEL® Sustained Release Formulation Administered Intravitreally or in the Sub-tenon Space in the Rabbit

Octreotide, a long-acting octapeptide with pharmacologic actions similar to the natural hormone, somatostatin, is a potential therapy for retinal and choroidal neovascularization. A polymeric, biodegradable in situ-forming implant (ATRIGEL® formulation) was developed for the sustained release of octreotide. The tolerance of the implants administered intravitreally or in the sub-tenon space in the rabbit eye and the release of octreotide to the posterior segment is reported hererin.

Part A:

Methods: A 12% octreotide/ATRIGEL formulation that displayed a 90-day release profile in subcutaneous tissue and the equivalent blank ATRIGEL formulation or saline were administered intravitreally (IVT) or in the sub-tenon (ST) space in New Zealand White rabbits. Tolerance was assessed by monitoring intraocular pressure and by ophthalmic examination up to 120 days. Eye globes were collected for histopathology at 30, 90 and 120 days after injection. Octreotide concentrations in ocular tissues and in implants retrieved at different timepoints were determined by LC-MS/MS.

Formulation: The formulation used in this example was 12% octreotide in 50% 85/15% PLGHP 0.27 in NMP. A control formulation without octreotide was also used. The 3-mo formulation can be used to treat, for example, carcinoid syndrome and acromegaly.

Results: In 10% of 30 eyes, subtenon injections of both the octreotide/ATRIGEL formulations and blank ATRIGEL formulations were associated with conjunctival swelling and hyperemia that resolved after 1 week. No adverse effects were observed after intreavitreal administration, except procedure-related cataracts in less than 10% of the eyes. The octreotide/ATRIGEL formulation exhibited a 24-hour release of 18% and 22% after intravitreal and sub-tenon administration, respectively, compared to 20% after subcutaneous injection. The release rate fitted a zero-order kinetic with about 50% and 75% of octreotide released from intravitreal and subtenon implants, respectively, 45 days after injection. Biodistribution results indicated that octreotide concentrations in the retina and choroid were similar after intravitreal administration, whereas the retina concentrations were about 10 times lower than those in the choroid in eyes with subtenon implants.

Conclusions: The ATRIGEL delivery system is well tolerated in the eye and can effectively deliver octreotide to the retina and choroid. The release of octreotide from intravitreal or sub-tenon implants was consistent with that observed in subcutaneous implants.

Part B:

Injection procedure: Eyes were prepared for dose administration as follows: Approximately 20 minutes prior to dosing, eyes were dilated with 1-2 drops of 1% Tropicamide and 1-2 drops of 2.5% Phenylephrine hydrochloride. About five minutes prior to dosing, eyes were moistened with an ophthalmic Betadine solution. After five minutes, the Betadine was washed out of the eyes with sterile saline. Proparacaine hydrochloride 0.5% (1-2 drops) was delivered to each eye.

Intravitreal Dosing Procedure: On Day 1, each eye received a 25-μL intravitreal injection of test article, control article, or saline, as described in the treatment group table. Intravitreal injections were given using a Hamilton 50-μL glass syringe with Teflon plunger and 25-gauge sharp metal-hub needle. For each injection, the needle was introduced from a conjunctival site temporal to the dorsal rectus muscle, approximately 2-3 mm posterior to the limbus, with the bevel of the needle directed downward and posteriorly to avoid the lens. Test or control article was injected in a single bolus at a location roughly in the center of the vitreous. The needle was rotated as it was removed following injection.

Sub-Tenon's Dosing Procedure: On Day 1, the appropriate eyes received a 100-μL sub-Tenon's capsule injection of test article (octreotide ATRIGEL®) or one of two control articles (0.9% NaCl or ATRIGEL®), as described in the treatment group table (Table 7-1). Injections were given using a Hamilton 250-μL glass syringe with Teflon plunger and blunt, curved 22-gauge sub-Tenon's cannula. For each injection, a guide hole was made in the dorsotemporal quadrant 3 mm posterior to the limbus and temporal to the dorsal rectus muscle attachment with a 20-gauge sharp needle, and the surgeon guided the cannula posteriorly into the sub-Tenon's space such that the implant was placed on the posterior surface of the sclera near the optic nerve.

For all injections, the weight of the syringe, before and after dosing, was recorded. The time of injection was also recorded. The test articles were explanted from the appropriate eyes at different timepoints, placed into 20-mL scintillation vials and stored at −70° C., before being processed as described below.

OTCA Atrigel® Formulation Extraction Method:

1.) The OTCA/ATRIGEL implants were collected in labeled scintillation vials. 2.) The retrieved implants were frozen at −70° C. for at least 1 hour in a freezer. 3) Once frozen, the samples are then freeze-dried for at least 4 hours or until dry. 4.) The samples were minced prior to extraction to aid dissolution. 5) 5 mL of extraction solvent (70:30 DMSO:methanol+1% PEI) was added to each sample using a micropipette. 6.) All samples were mixed overnight at 37° C. on an orbital shaker (200 rpm). 7.) After overnight incubation, the samples were solicated for 10 minutes at room temperature. 8.) All samples were clarified by filtering through 0.2 μm pore size membrane. 9.) 1 mL of filtrate was then diluted with 4 mL of dilution solvent (1:1 ACN:water). 10.) The samples were vortexed until no phase separation was observed. 11.) The diluted extracts were filtered through 0.2 μm pore size membrane, directly into HPLC vials and capped. 12.) All extracts were analyzed for octreotide content by high performance liquid chromatography (HPLC).

HPLC Method And Conditions:

TABLE 7-1
Octreotide HPLC method

| | |
|---|---|
| HPLC Column | Symmetry C18 4.6 × 150 3.5 μm |
| Mobile Phase | 80:20 0.1% TFA/0.1% TFA in |
| | ACN |
| Flow Rate | 1.5 mL/min |
| Detection Wavelength | 210 nm |
| Injection Volume | 5 μl |
| Column Temperature | 35°C. |
| Sample Temperature | 4°C. |
| Run Time | 15 min |
| Retention Time of OTCA | ~11 min |

Octreotide Atrigel® 24-Hour Release after Subcutaneous Injections (for Comparison)

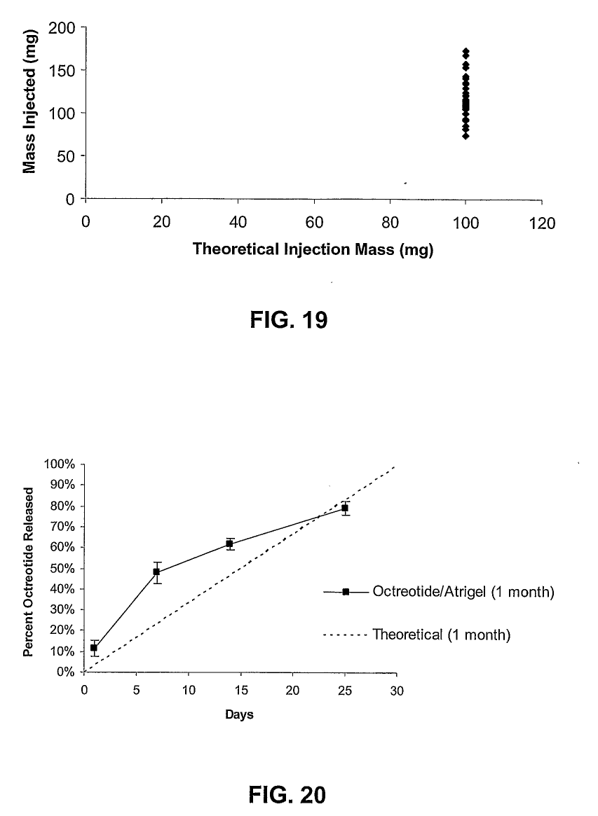

FIG. 19 illustrates the weight distribution of Octreotide ATRIGEL® SC injections. FIG. 20 illustrates the extended release of Octreotide ATRIGEL® from SC implants. Table 7-2 shows the results of extended release of octreotide from ATRIGEL® implants after SC injections.

TABLE 7-2
Results of Extended Release of Octreotide from ATRIGEL ®
implants after SC injections

| Formulation | Mean ± SD |
|---|---|
| 1-month Octreotide ATRIGEL ® - Day 1 | 11.4% ± 3.9% |
| 1-month Octreotide ATRIGEL ® - Day 7 | 47.8% ± 5.3% |
| 1-month Octreotide ATRIGEL ® - Day 14 | 61.8% ± 2.8% |
| 1-month Octreotide ATRIGEL ® - Day 25 | 79.2% ± 3.0% |

### 4-Month Evaluation of Octreotide Distribution and Safety Following Ivt or Subtenon Injection In New Zealand White Rabbits (Btc Protocol)

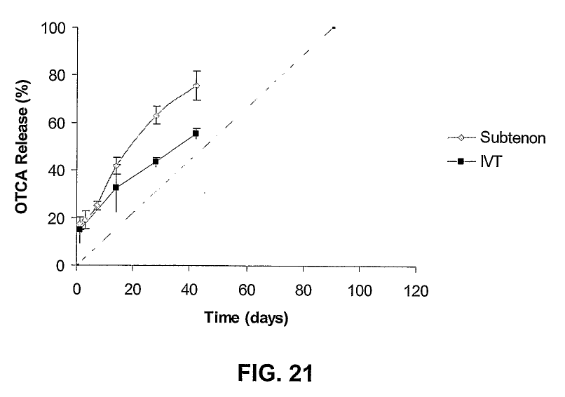

FIG. 21 shows the results of release of Octreotide ATRIGEL® from IVT and ST implants. Table 7-3 shows the corresponding study results.

TABLE 7-3
Study Results: Release of Octreotide ATRIGEL ® from IVT
and ST implants

| | Mean of IVT | Mean of ST |
|---|---|---|
| Formulation | Release (%) ± SD | Release (%) ± SD |
| 3-month Octreotide ATRIGEL ® - | 14.8% ± 5.4%^{a} | 17.6% ± 2.5% |
| Day 1 | | |
| 3-month Octreotide ATRIGEL ® - | — | 19.0% ± 3.7% |
| Day 3 | | |
| 3-month Octreotide ATRIGEL ® - | — | 25.1% ± 1.8% |
| Day 7 | | |
| 3-month Octreotide ATRIGEL ® - | 32.4% ± 9.8% | 41.7% ± 3.5% |
| Day 14 | | |
| 3-month Octreotide ATRIGEL ® - | 43.4% ± 2.0% | 63.2% ± 3.6% |
| Day 28 | | |
| 3-month Octreotide ATRIGEL ® - | 55.4% ± 2.2% | 75.8% ± 6.0% |
| Day 42 | | |

### Example 8

### OTCA Subcutaneous Size vs. Burst

A “1 Month” formulation was used for this study: 12% OTCA in 20% 85/15 PLGHp InV 0.27, 30% 50/50 PLGH InV 0.30 in NMP. One hundred μL Hamilton Syringes and 19 G special thin-walled needles were used for all injections. Implant sizes: 100 μL, 50 μL, 25 μL, 10 μL.

Table 8-1 shows the percent burst for different implant sizes.

TABLE 8-1
Percent burst with respect to Implant Size.

| Group | Implant size (μL) | Burst (%) | SD (%) |
|---|---|---|---|
| 1 | 100 μL | 11.8% | 2.4% |
| 2 | 50 μL | 9.2% | 2.1% |
| 3 | 25 μL | 14.5% | 2.9% |
| 4 | 10 μL | 14.6% | 7.6% |

### Example 9

### Octreotide Formulations

Octreotide can be delivered to a patient by several different methods. Described in this example are seven formulations in which octreotide can be delivered in a sustained release formulation. Table 9-1 identifies seven octreotide formulations for clinical studies.

TABLE 9-1
Octreotide Clinical Formulations

| | Strength (mg | Release |
|---|---|---|
| Formulation | of octreotide | Duration |
| Identifier | delivered) | (months) |
| A | 20 | 1 |
| B | 20 | 1 |
| C | 30 | 1 |
| D | 60 | 3 |
| E | 60 | 3 |
| F | 60 | 3 |
| G | 90 | 3 |

Table 9-2 lists the components of the delivery systems for the seven octreotide formulations. Each of these delivery systems is prepared in the same manner. The polymer and N-methyl-pyrrolidone (NMP) are weighed out in the correct proportion and then agitated until a solution is formed. This solution is then filled into a syringe with a female luer lock end with a tightly controlled fill weight. This syringe is then placed into a secondary package like a pouch or tray that is then sealed.

TABLE 9-2
Delivery System Components and Fill

| | Polymer | | | |
|---|---|---|---|---|
| | Composition | | | |
| | and Nominal | Polymer | | |
| Formulation | Molecular | Content | NMP Content | Target Fill |
| Identifier | Weight¹ | (% w/w) | (% w/w) | Weight (mg) |
| A | 50/50 PLGH | 37 | 63 | 244 |
| | 36 kDa | | | |
| B | 55/45 PLGH | 30 | 50 | 295 |
| | 28 kDa | | | |
| | 85/15 PLGH | 20 | | |
| | 25 kDa | | | |
| C | 55/45 PLGH | 30 | 50 | 410 |
| | 28 kDa | | | |
| | 85/15 PLGH | 20 | | |
| | 25 kDa | | | |
| D | 85/15 PLGH | 50 | 50 | 710 |
| | 29 kDa | | | |
| E | 90/10 PLGH | 50 | 50 | 710 |
| | 25 kDa | | | |
| F | 95/05 PLGH | 45 | 45 | 710 |
| | 34 kDa | | | |
| G | 85/15 PLGH | 50 | 50 | 1014 |
| | 25 kDa | | | |

¹Weight average molecular weight determined by gel permeation chromatography using polystyrene molecular weight standards.

The packaged syringe is then sterilized by gamma irradiation. This is typically done at a dose of 18 to 28 kiloGrays. The dose must be sufficient to reach the desired sterility assurance level but not so high that the polymer molecular weight is decreased more than seen with this dose level. The final package must maintain the sterility of its contents and limit transmission of water into the package.

Table 9-3 lists the contents of the drug syringes for the same seven octreotide formulations. Each of these drug syringes are prepared in the same manner. A bulk solution of octreotide acetate and citric acid in water is prepared. The solution is sterile filtered and filled (with a tightly controlled fill weight) into a syringe barrel with a male luer lock end that is capped. The filled syringes are then lyophilized to give a dry cake in the syringe. The syringe is then stoppered. All steps from filtration through stoppering are done aseptically to ensure the sterility of the syringe contents. The filled syringe is then placed in a secondary package such as a pouch or tray that is then sealed.

TABLE 9-3

| Formulation | Octreotide¹ | Citric Acid |
|---|---|---|
| Identifier | Weight (mg) | Weight (mg) |
| A | 30 | 6.8 |
| B | 32 | 6.6 |
| C | 40 | 9.1 |
| D | 79 | 16.2 |
| E | 79 | 16.2 |
| F | 79 | 16.2 |
| G | 110 | 22.5 |

¹This is the weight of the octreotide peptide. The amount of octreotide acetate used must be adjusted based on the potency of the salt. There is residual acetic acid in the lyophilized powder.

Table 9-4 lists the nominal contents of what can be delivered to the patient for the seven formulations. This is the result of coupling a drug syringe with a delivery syringe and passing the syringe contents back and forth to constitute the actual formulation. The product is held in the male luer ended syringe and the syringes are decoupled. A needle can then be placed on the syringe and a subcutaneous injection of the syringe contents may be given. The total amount of material delivered will be less than in the filled syringes because of material that is held up in the syringes and in the needle.

TABLE 9-4
Nominal Delivered Amounts in the Formulation

| Formulation | Octreotide¹ | | | NMP |
|---|---|---|---|---|
| Identifier | (mg) | Citric Acid (mg) | Polymer (mg) | (mg) |
| A | 20 | 4.6 | 60 | 103 |
| B | 20 | 4.1 | 93 | 93 |
| C | 30 | 6.1 | 139 | 139 |
| D | 60 | 12 | 285 | 285 |
| E | 60 | 12 | 285 | 285 |
| F | 60 | 12 | 257 | 314 |
| G | 90 | 18 | 427 | 427 |

¹This is the weight of the octreotide peptide.

The polymer for formulation G is from Boehringer Ingelheim and was purified by the manufacturer to have very low monomer content. All the other polymers are purified by precipitation of a dichloromethane solution in methanol. These polymers are synthesized and purified by QLT USA.

### REFERENCES

1. Ambati, J. et al: (2003) Age-Related Macular Degeneration: Etiology, Pathogenesis, and Therapeutic Strategies. Survey of Opthalmology International Review Journal 48 (3), 257-293;
2. Barnett, P.: (2003) Somatostatin and Somatostatin Receptor Physiology. Endocrine 20 (3), 255-264;
3. Benali, N. et al: (2000) Somatostatin Receptors. Digestion 62 (Suppl 1) 27-32;
4. Benson, W. E.: (1999) Vascular Disorders: Diabetic Retinopathy.

Opthalmology, Retina and Vitreous, Section 8, Chapter 20, 20.1-21.10;
5. Berger, J. W. et al. (1999) Age-Related Macular Degeneration. Mosby, Inc.;
6. Boehm, B. O., et al: (2001) Octreotide Reduces Vitreous Hemorrhage and Loss of Visual Acuity Risk in Patients with High-Risk Proliferative Diabetic Retinopathy. Horm Metab Res 33, 300-306;
7. Buchan, A. et al: (2002) Somatostatin, Acting at Receptor Subtype 1, Inhibits Rho Activity, the Assembly of Actin Stress Fibers, and Cell Migration. The Journal of Biological Chemistry 277 (32), 28431-28438;
8. Celorio, J. M.: (1999) Macular Disorders: Chorodial Neovascularization. Opthalmology, Retina and Vitreous, Section 8, Chapter 27, 21.7.1-27.4;
9. Culler, M. D., et al: (2002) Somatostatin Receptor Subtypes: Targeting Functional and Therapeutic Specialty. Ann. Endocrinol 63 (2) Cahier 3, 255-2512;
10. de la Torre, N. et al: (2002) Antiangiogenic Effects of Somatostatin Analogues. Clinical Endocrinology 75, 425-441;
11. Demir, T. et al: (1999) Effect of Octreotide on Experimental Corneal Neovascularization. Acta Opthalmologica Scandanavia 77, 386-390;
12. Durak M. D. et al: (1995) Somatostatin Receptors in the Orbits. Clinical Nuclear Medicine 20 (30 237-242;
13. Edwards, M. G. et al: (1999) Macular Disorders: Age-Related Macular Degeneration. Opthalmology, Retina and Vitreous, Section 8, Chapter 28, 28.1-28.10;
14. Ferris III, F. L. et al: (1999) Treatment of Diabetic Retinopathy. Massachusetts Medical Journal 341 (9), 667-678;
15. Fine, Stuart L., et al: (2000) Age-Related Macular Degeneration. Massachusetts Medical Society 342 (7), 483-492;
16. Frank, R. N., et al: (2004) Diabetic Retinopathy. New England Journal of Medicine 350, 48-58;
17. Grant, M. B. et al: (1993) Inhibition of IGF-I and b-FGF Stimulated Growth of Human Retinal Endothelial Cells by the Somatostatin Analogue, Octreotide: A potential Treatment for Ocular Neovascularization. Regulatory Peptides 48, 267-278;
18. Grant, M. B., et al: (2000) The Effect of Octreotide in the Therapy of Severe Nonproliferative and Early Proliferative Diabetic Retinopathy. Diabetes Care 23 (4), 504-509;
19. Grant, M. B., et al: (2002) Somatostatin Analogues as Drug Therapies for Retinopathy. Drugs of Today 38 (11), 783-791;
20. Johnson, J. et al: (2000) Somatostatin and Somatostatin Subtype 2A Expression in the Mammalian Retina. Microscopy Research and Technique 50, 103-111;
21. Kertes, P. J., et al: (1998) Clinical Trials in Opthalmology, A Summary and Practice Guide. Lippencott Williams & Wilkins;
22. Krassas, G. E. (1998) Somatostatin Analogues in the Treatment of Thyroid Eye Disease. Thyroid 8 443-445;
23. Kuijpers, R. et al: (1998) Treatment of Cystoid Macular Edema with Octreotide. The New England Journal of Medicine 338 (9), 624-626;
24. Lambooij, A. C., et at: (2000) Somatostatin Receptor 2A Expression in Choroidal Neovascularization Secondary to Age-Related Macular Degeneration. Investigative Opthalmology & Visual Science 41 (8) 2329-2335;
25. Mckeage, K, et al. Octreotide Long-Acting Release (LAR). (2003) A Review of its Use in the management of Acromegaly. Drugs, 63: 2473-2499;
26. Moller, L. N. et al: (2003) Somatostatin Receptors. Biochimica at Biophysica Acta 1616, 1-84;
27. Papdaki M. D. et al: (2003) The Role of Lanreotide in the Treatment of Choroidal Neovascularization Secondary to Age-Related Macular Degeneration, A Pilot Clinical Trial. Retina 23, 800-807;
28. Pasquali, D. et al: (2002) Somatostatin Receptor Genes Are Expressed in Lymphocytes from Tetroorbital Tissues in Graves' Disease. The Journal of Clinical Endocrinology & Metabolism 87 (11) 5125-5129; 29. Patel, Y. C.: (1999) Somatostatin and Its Receptor Family. Frontiers in Neuroendocrinology 20, 157-198;
30. Rothnova, A.: (2002) Medical Treatment of Cystoid Macular Edema. Ocular Immunology and Inflammation 10 (4), 239-246;
31. Schubert, H. D.: (1999) Anatomy and Physiology: Structure and Function of the Neural Retina. Opthalmology, Retina and Vitreous, Section 8, Chapter 1, 1.1-1.4;
32. Shimon, I.: (2003) Somatostatin Receptors in Pituitary and Development of Somatostatin Receptor Subtype-Selective Analogs. Endocrine 20 (3), 265-269;
33. Spraul, C. W. et al: (2002) Octreotide inhibits growth factor-induced bovine choriocapillary enthelial cells in vitro. Graefe's Ach Clin Exp Opthalmol 240, 227-231;
34. van Bijsterveld, O. P.: (2000) Diabetic Retinopathy. Martin Dunitz, Ltd.;
35. Watson, J. C., et al: (2001) Growing Vascular Endothelial Cells Express Somatostatin Subtype 2 Receptors. British Journal of Cancer 85(2), 266-272;
36. Woltering, E. A. et al: (2003) Development of Targeted Somatostatin-Based Antiangiogenic Therapy: A Review and Future Perspectives. Cancer Biotherapy & Radiopharmaceuticals 18, 601-609.

All patents and publications referenced or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety. Applicants reserve the right to physicall incorporate into this specification any and all materials and information from any such cited patents or publications.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the claims.

What is claimed is:

1. A flowable composition comprising:
(a) a biodegradable thermoplastic polymer that is at least substantially insoluble in body fluid;
(b) a biocompatible polar aprotic organic liquid; and
(c) octreotide.

2. A flowable composition of claim 1 wherein the organic liquid is selected from the group consisting of an amide, an ester, a carbonate, a ketone, an ether, and a sulfonyl; and wherein the biocompatible polar aprotic liquid has a solubility in aqueous medium or body fluid ranging from insoluble to completely soluble in all proportions.

3. The composition of claim 1 wherein the biodegradable thermoplastic polymer is a polyester of one or more hydroxy carboxylic acids, or is a polyester of a combination of one or more diols and one or more dicarboxylic acids.

4. The composition of claim 3 wherein the hydroxy carboxylic acid or acids are in the form of dimers.

5. The composition of claim 4 wherein the polyester is a polylactide, a polyglycolide, a polycaprolactone, a copolymer thereof, a terpolymer thereof, or any combination thereof.

6. The composition of claim 3 wherein the biodegradable thermoplastic polyester is a 50/50, 55/45, 75/25, 85/15, 90/10, or 95/5 poly (DL-lactide-co-glycolide) having a carboxy terminal group, or is a 50/50, 55/45, 75/25, 85/15, 90/10, or 95/5 poly (DL-lactide-co-glycolide) without a carboxy terminal group, and optionally the polyester without a terminal carboxyl group is extended with a diol.

7. The composition of claim 3 wherein the biodegradable thermoplastic polyester is present in about 20 wt. % to about 90 wt. %, or about 30 wt. % to about 70 wt. % of the composition, and optionally the biodegradable thermoplastic polyester has an average molecular weight of from about 15,000 to about 45,000 Daltons, preferably about 20,000 to about 40,000 Daltons.

8. The composition of claim 1 wherein the biocompatible polar aprotic liquid is N-methyl-2-pyrrolidone, 2-pyrrolidone, N,N-dimethylformamide, dimethyl sulfoxide, propylene carbonate, caprolactam, triacetin, or any combination thereof, and preferably the biocompatible polar aprotic liquid is N-methyl-2-pyrrolidone.

9. The composition of claim 1 wherein the biocompatible polar aprotic liquid is present in about 10 wt. % to about 90 wt. % of the composition, or preferably the biocompatible polar aprotic liquid is present in about 30 wt. % to about 70 wt. % of the composition.

10. The composition of claim 1 wherein the octreotide is present in about 0.001 wt. % to about 10 wt. % of the composition, or preferably the octreotide is present in about 1 wt. % to about 8 wt. % of the composition.

11. The composition of claim 1 that is an injectable subcutaneous formulation, and optionally has a volume of about 0.20 mL to about 2 mL, or preferrably has a volume of about 0.30 mL to about 1 mL.

12. The composition of claim 11 that is formulated for administration about once per month, or preferably is formulated for administration about once per three months, or more preferably is formulated for administration about once per four months to about once per six months.

13. A method for forming a flowable composition for use as a controlled release implant, comprising the step of mixing, in any order:
(a) a biodegradable thermoplastic polymer that is at least substantially insoluble in aqueous medium or body fluid;
(b) a biocompatible polar aprotic liquid; and
(c) octreotide;
wherein the mixing is performed for a sufficient period of time effective to form the flowable composition for use as a controlled release implant.

14. The method of claim 13 wherein the biocompatible thermoplastic polymer and the biocompatible polar aprotic liquid are mixed together to form a mixture and the mixture is then mixed with the octreotide to form the flowable composition.

15. A biodegradable implant formed in situ, in a patient, by the steps comprising:
(a) injecting a composition of claim 1 into the body of the patient; and
(b) allowing the biocompatible polar aprotic liquid to dissipate to produce a solid or gel biodegradable implant.

16. A biodegradable implant according to claim 15 wherein the composition comprises an effective amount of the biodegradable thermoplastic polymer; an effective amount of the biocompatible polar aprotic liquid; and an effective amount of octreotide, and wherein the solid implant releases an effective amount of octreotide over time as the solid implant biodegrades in the patient and optionally the patient is a human.

17. A method of forming a biodegradable implant in situ, in a living patient, comprising the steps of:
(a) injecting the flowable composition of claim 1 into the body of a patient; and
(b) allowing the biocompatible polar aprotic liquid to dissipate to produce the solid or gel biodegradable implant

18. The method of claim 17 wherein the solid biodegradable implant releases the effective amount of octreotide by diffusion, erosion, or a combination of diffusion and erosion as the implant biodegrades in the patient.

19. A flowable composition of claim 1 wherein the octreotide is in the form of a salt and the salt gegenion is derived from a pharmaceutically acceptable organic or inorganic acid, or preferably the gegenion is a polycarboxylic acid.

20. A flowable composition of claim 1 having the property of production of minimal tissue necrosis when injected subcutaneously.

21. A kit comprising:
(a) a first container comprising a composition comprising a biodegradable thermoplastic polymer that is at least substantially insoluble in or body fluid and a biocompatible polar aprotic liquid; and
(b) a second container comprising octreotide, and wherein optionally the first container is a syringe, and optionally the second container is a syringe, and optionally the octreotide is lyophilized, and optionally the kit further comprises instructions, and optionally the first container can be connected to the second container, or optionally the first container and the second container are each configured to be directly connected to each other.

22. An implant comprising:
(a) a biocompatible thermoplastic polymer that is at least substantially insoluble in aqueous medium or body fluid; and
(b) octreotide; and,
wherein the implant has a solid or gel monolithic structure.

23. An implant according to claim 22 wherein the implant has a solid or gelatinous matrix, the matrix being a core surrounded by a skin.

24. An implant according to claim 23 wherein the implant is solid and is microporous.

25. The solid implant of claim 22 further comprising a biocompatible organic liquid that is very slightly soluble to completely soluble in all proportions in body fluid and at least partially dissolves at least a portion of the thermoplastic polyester, and optionally the amount of biocompatible organic liquid is less than about 5 wt. % of the total weight of the implant, and optionally the amount of biocompatible organic liquid decreases over time.

26. The solid implant of claim 24 wherein the core contains pores of diameters from about 1 to about 1000 microns, and optionally the skin contains pores of smaller diameters than those of the core pores, and optionally the skin pores are of a size such that the skin is functionally non-porous in comparison with the core.

27. A flowable composition of claim 1 having a substantially linear cumulative release profile.

28. A method for treatment of a patient having a malcondition associated with somatotropin hypersecretion, gastrointestinal syndrome, with an imbalance, hyper or hypo activity of an insulin, glucagon or somatotropin pathway, or with a somatotropin or somatostatin receptor function, comprising administering to the patient an effective amount of octreotide in combination with an at least substantially water-insoluble biodegradable thermoplastic polymer and a biocompatible, polar aprotic organic liquid, or preferably the malcondition is associated with diabetes, cardiovascular failure or abnormal performance, angiopathy, carcinoid syndrome, somatotropin or somatostatin receptor associated cancer, and more preferably the malcondition is a proliferative eye disease, a neovascular proliferative eye disease or a diabetic eye disease.

29. A method for treatment of a patient having diabetic retinopathy comprising administering to the patient an effective amount of octreotide in combination with an at least substantially water-insoluble biodegradable thermoplastic polymer and a biocompatible, polar, aprotic organic liquid.

30. A method for treatment of a patient having carcinoid syndrome comprising administering to the patient an effective amount of octreotide in combination with an at least substantially water-insoluble biodegradable thermoplastic polymer and a biocompatible, polar, aprotic organic liquid.

31. A method for treatment of a patient according to claim 28 further comprising a combination therapy with another known pharmaceutical compound designated for treatment of the malcondition.
